(19) United States
(12) Patent Application Publication
(10) Pub. No.: US 2024/0398800 A1
(43) Pub. Date: Dec. 5, 2024

(54) COMPOUNDS FOR TARGETED DEGRADATION OF TAF1

(71) Applicant: H. LEE MOFFITT CANCER CENTER AND RESEARCH INSTITUTE, INC., Tampa, FL (US)

(72) Inventors: Justin LOPCHUK, Tampa, FL (US); Ernst SCHONBRUNN, Tampa, FL (US); Jiandong CHEN, Tampa, FL (US); Zachary SHULTZ, Tampa, FL (US)

(21) Appl. No.: 18/690,403

(22) PCT Filed: Sep. 12, 2022
(86) PCT No.: PCT/US2022/076277

§ 371 Date: Mar. 8, 2024

Related U.S. Application Data
(60) Provisional application No. 63/242,725, filed on Sep. 10, 2021.

(51) Int. Cl. A61K 31/506; A61K 31/437; A61K 31/444; A61P 35/00; C07D 471/04

(52) U.S. Cl. CPC A61K 31/506; A61K 31/437; A61K 31/444; A61P 35/00; C07D 471/04

(57) ABSTRACT

This present disclosure provides compounds which are useful in treating medical disorders. More particularly, compounds are provided for targeted degradation of TAF1 which are useful in the treatment of cancers.

### CROSS-REFERENCE TO RELATED APPLICATIONS

This application claims the benefit of priority to U.S. Provisional Application No. 63/242,725, filed Sep. 10, 2021, the disclosure of which is incorporated herein by reference in its entirety.

### TECHNICAL FIELD

This disclosure relates to compounds which are useful in treating medical disorders, and more particularly to compounds for targeted degradation of TAF1 which are useful in the treatment of cancers.

### BACKGROUND

Transcription initiation by RNA polymerase II requires more than 70 polypeptides, with the basal transcription factor TFIID being the protein that coordinates this activity. TFIID binds to the core promoter to position the polymerase properly, serves as the scaffold for assembly of the remainder of the transcription complex, and acts as a channel for regulatory signals. TFIID is composed of the TATA-binding protein (TBP) and a group of proteins known as TBP-associated factors, or TAFs, which may participate in basal transcription, serve as coactivators, function in protomer recognition, or modify general transcription factors (GTFs) to facilitate complex assembly and transcription initiation. The TAF1 gene encodes the largest subunit of TFIID. Full-length TFA1 is composed of several domains, including a tandem bromodomain (BRD) module, which performs a wide range of regulatory functions in transcription (see Louder, R. K. et al., Nature 2016, 531-604-609; Wang, H. et al., Cell Res 2014, 1433-1444; Bhattacharya, S. et al., Proc Natl Acad Sci USA 2014, 111, 9103-9108).

TAF1 protein has been shown to play various roles in oncological disorders. TAF1 binds to Myc oncoprotein and assists in Myc-driven gene transcription (see Wei, Y. et al., Nat Struct Mol Biol 2019, 26, 1035-1043). TAF1 BRD directly interacts with acetylated p53 to initiate the transcription of p53 target genes (see Li, A. G. et al., Molecular Cell 2007, 28, 408-421). TAF1 activates Mdm2-mediated p53 degradation leading to GUS cell cycle transition (see Li, H. et al., Molecular Cell 2004, 13, 867-878; Allende-Vega, N. et al., Oncogene 2007, 26, 4234-4242; Cai, X. et al., Proc Natl Acad Sci USA 2008, 105, 16958-16963). Cell lines with defective TAF1 exhibit hallmarks of an ATR-mediated DNA damage response (see Buchmann, A. M. et al., Mol Cell Bio 2004, 24, 5332-5339). TAF1 is found to be significantly mutated in uterine serous carcinoma (see Hong, B. et al., Curr Opin Genet Dev 2015, 30, 25-31), TAF1 overexpression is a major factor for the high mitotic activity of solid tumors (see Wada, C. et al., Cancer Res 1992, 52, 307-313), and TAF1 BRD function has been implicated in AML1-ETO driven acute myeloid leukemia (see Wang, L. et al., Science 2011, 333, 765-769; Xu, Y. et al., Nat Commun 2019, 10, 4925).

Several TAF1 BRD inhibitors have been reported, with the most potent to date being BAY299 and GNE-371; however, cellular studies are lacking or inconclusive, and no TAF1 inhibitor has yet to reach the clinic (see Foote, K. M. et al., J Med Chem 2018, 61, 9889-9907; Bouche, L. et al., J Med Chem 2017, 60, 4002-4022; Wang, S. et al., J Med Chem 2018, 61, 9301-9315).

There is a clear need for the development of compounds which target the activity of TAF1, particularly in view of the potential effect in the treatment of oncological disorders. The present disclosure addresses this as well as other needs.

### SUMMARY

The present disclosure provides compounds which facilitate the targeted degradation of the TAF1 protein. The compounds described herein are therefore useful in the treatment of medical disorders, in particular cancers.

Thus, in one aspect, a compound is provided of Formula I, Formula II, or Formula III:

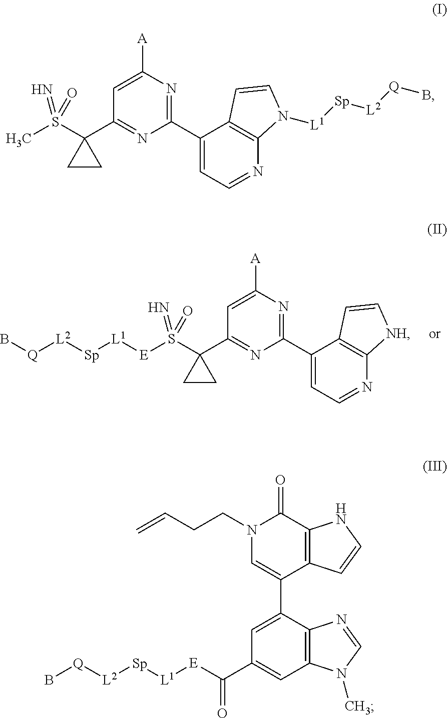

or a pharmaceutically acceptable salt thereof, wherein all variables are as defined herein.

In another aspect, a compound of Formula IV is provided:

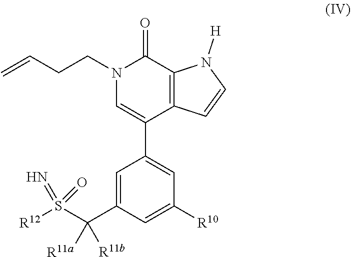

or a pharmaceutically acceptable salt thereof, wherein all variables are as defined herein.

In another aspect, a pharmaceutical composition is provided comprising a compound described herein and a pharmaceutically acceptable carrier.

In a further aspect, a method is provided for treating a cancer in a subject in need thereof comprising administering to the subject a therapeutically effective amount of a compound described herein, or a pharmaceutically acceptable salt, or a pharmaceutical composition as described herein.

The details of one or more embodiments of the disclosure are set forth in the accompanying drawings and the description below. Other features, objects, and advantages of the disclosure will be apparent from the description and the claims.

### DESCRIPTION OF DRAWINGS

FIGS. 1A-1C show the induction of TAF1 degradation by PROTAC and induction of apoptosis. (FIG. 1A) TAF1 PROTAC compounds induce target degradation with DC₅₀ in the nM range. AML cell line HL60 was treated with indicated compounds and analyzed for TAF1 level by Western blot. (FIG. 1B) TAF1 PROTAC induces sustained target degradation for >24 hrs after compound washout. UC3 bladder cancer cells were treated with TAF1 PROTAC for 2 hrs. The compound was removed by washing 3 times. The cells were collected at 4-48 hrs after compound washout and analyzed for TAF1 level by Western blot. (FIG. 1C) TAF1 PROTACs induce apoptosis of AML and solid tumor cells. AML and osteosarcoma cell lines were treated with PROTAC compounds for 18 hrs and analyzed for TAF1 degradation and PARP cleavage (apoptosis) by Western blot. ZS1-959 is a negative control PROTAC (N-methylated).

FIGS. 2A-2B show that TAF1 degradation by PROTAC induces cell death and inhibits clonogenic growth. (FIG. 2A) TAF1 PROTACs induce apoptosis of AML and osteosarcoma cells. The cell lines were treated with TAF1 PROTAC compounds for 24 hrs. Cell death was documented by microscopy. (FIG. 2B) Short-term treatment with TAF1 PROTAC inhibits long-term survival. Bladder cancer cell lines were seeded in 24 well plate at 2000 cells/well and treated with 100 nM TAF1 PROTAC for 16 hrs. The cells were washed 3 times to remove the compound, and cultured for 10 days. Colony formation was detected by crystal violet staining.

FIGS. 3A-3C show that TAF1 PROTACs act through expected mechanisms. (FIG. 3A) Verification of TAF1 PROTAC compound mechanism. Kasumi-1 cells were treated with active TAF1 PROTAC compound in the presence of indicated competitors for 16 hrs. Inhibition of TAF1 degradation was determined by Western blot. Bortezomib rescue of TAF1 level indicates proteasome-dependent degradation mechanism. Rescue by Pomalidomide indicates dependence on Cereblon recruitment. Rescue by AZD6738, GNE371 and BAY299 indicates dependence on warhead binding to TAF1 bromodomain. (FIG. 3B) Mutation of TAF1 bromodomain blocks degradation by PROTAC. Saos2 cells were stably transfected with HA-TAF1 expression plasmid with N1583A mutation designed to block warhead binding. The cells were treated with TAF1 PROTAC for 5 hrs. Total TAF1 and ectopic HA-TAF1 levels were determined by Western blot, confirming the resistance of N1583A mutant to degradation. (FIG. 3C) TAF1 degradation leads to cell death. Saos2 cells stably transfected with HA-TAF1 expression plasmids were seeded at 20% density and treated with TAF1 PROTAC for 7 days, relative cell survival were documented by phase contrast microscopy. Non-degradable TAF1-N1583A mutant blocks cell death by TAF1 PROTAC, confirming the PROTAC kills cells by degrading TAF1.

FIGS. 4A-4B show that TAF1 PROTAC has potent growth inhibition activity against AML cells. AML cell lines were treated with TAF1 PROTAC ZS3-025 for 73 hrs at indicated concentrations. Cell viability was determined by incubation with MTS reagent for 3 hrs and measuring OD490. Controls were treated with 1:1 mixture of monovalent TAF1 and CRBN binding warheads at indicated concentrations. ZS3-025 inhibited cell viability with IC50 of 1-2 nM.

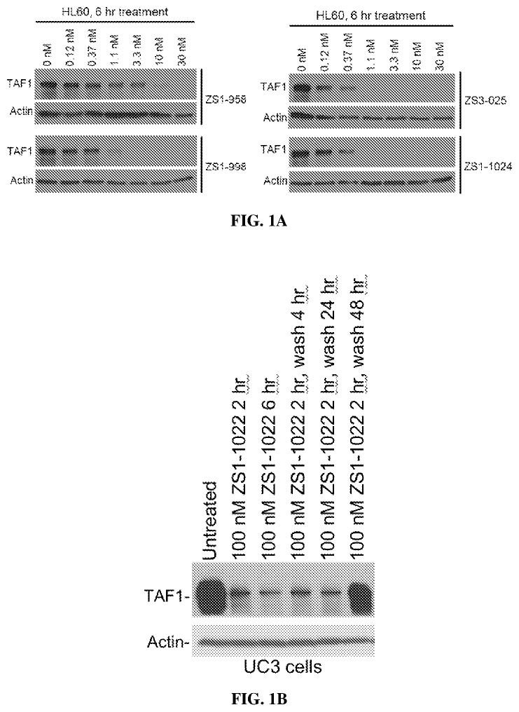

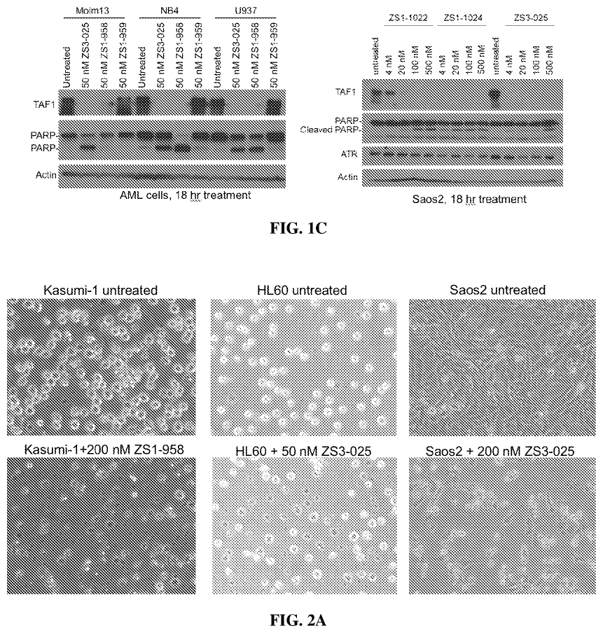

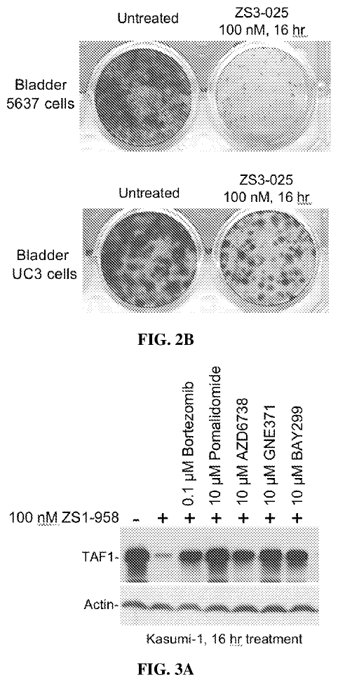

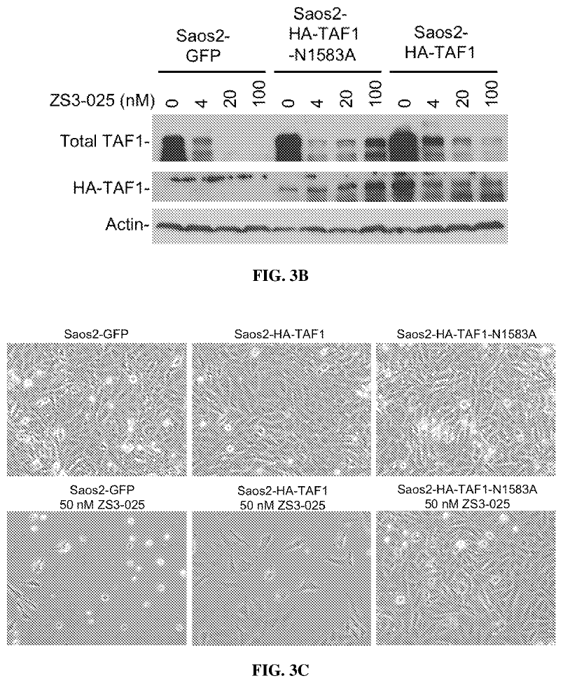

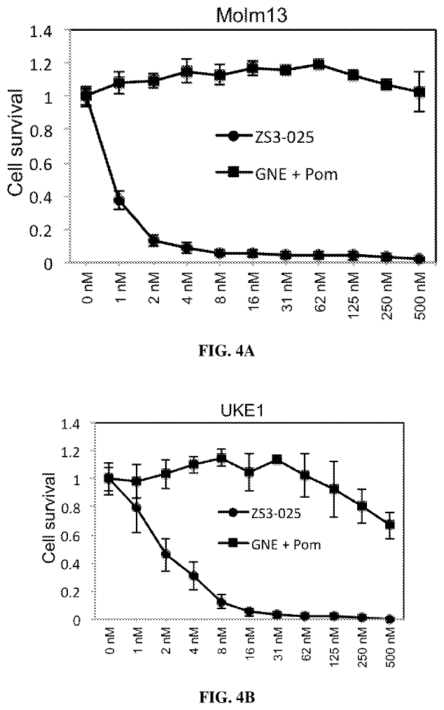

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an oncological disorder. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙ alkyl is used herein in conjunction with another group, for example (C₃-C₇cycloalkyl)C₀-C₄alkyl, or —C₀—C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀—C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═0)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

The present disclosure provides compounds which find use for the targeted degradation of the protein TAF1. The presently disclosed compounds are useful in the treatment of medical disorders, in particular cancers.

Thus, in one aspect, a compound is provided of Formula I, Formula II, or Formula III:

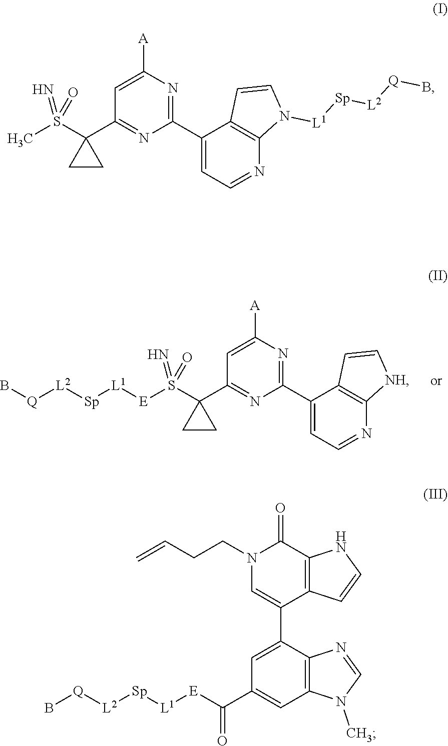

or a pharmaceutically acceptable salt thereof; wherein:
A is selected from:

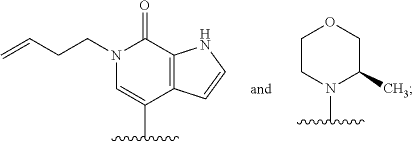 [SMILES: *c1cn(CCC=C)c(=O)c2[nH]ccc12 ; *N1CCOC[C@H]1C]

B is an E3 ubiquitin ligase-recruiting moiety;
L¹ and L² are independently selected from C₁-C₁₀ alkyl, C₂-C₁₀ alkenyl, C₂-C₁₀ alkynyl, and

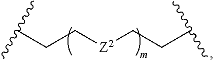

each of which may be substituted with one or more groups independently selected from X as allowed by valency;

Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl,

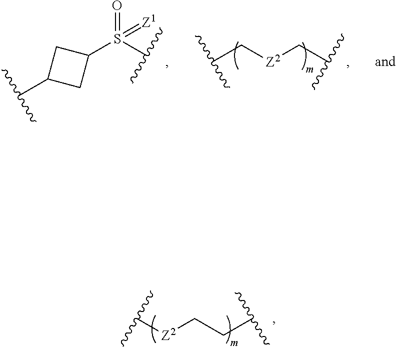

and “each of which may be optionally substituted with one or more groups independently selected from X as allowed by valency;
m is independently selected at each occurrence from 1, 2, 3, 4, 5, 6, 7, 8, 9, or 10;
Q is a bond or selected from and

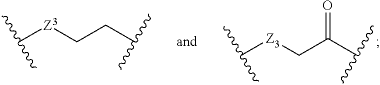

E is a bond or selected from —N(R^{a})— and
Z¹ and Z² are independently selected at each occurrence from 0 or N(R^{a});
Z³ is selected from CH₂, O, N(R^{a}), and S;
Z⁴ is selected from CH and N;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 6-membered heterocyclyl)-(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more groups selected from Y as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments of Formula I or Formula II, A is

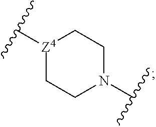

In some embodiments of Formula I or Formula II, A is

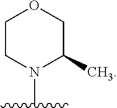 [SMILES: *N1CCOC[C@H]1C]

In another aspect, a compound of Formula IV is provided:

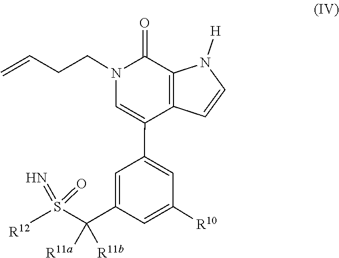

or a pharmaceutically acceptable salt thereof;
wherein:
R¹⁰ is selected from R^{10a} and —NH—R^{10a};
R^{10a} is selected from aryl, heteroaryl, cycloalkyl, or heterocycloalkyl, each of which may be optionally substituted with one or more groups independently selected from X as allowed by valency;
R^{11a} and R^{11b} are brought together with the carbon to which they are attached to form a cycloalkyl or heterocycloalkyl ring which may be optionally substituted with one or more groups independently selected from X as allowed by valency;
R¹² is selected from R^{12a} and —NH—R^{12b};
R^{12a} is selected from C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;
R^{12b} is selected from hydrogen C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;

wherein at least one of R¹⁰ or R¹² is substituted with B-Q-L²-Sp-L¹-E-;

B is an E3 ubiquitin ligase-recruiting moiety;

L¹ and L² are independently selected from C₁-C₁₀ alkyl, C₂-C₁₀ alkenyl, C₂-C₁₀ alkynyl, and

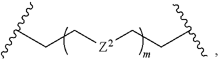

each of which may be substituted with one or more groups independently selected from X as allowed by valency;

Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl,

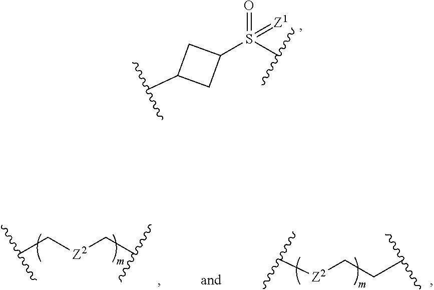

each of which may be optionally substituted with one or more groups independently selected from X as allowed by valency;
m is independently selected at each occurrence from 1, 2, 3, 4, 5, 6, 7, 8, 9, or 10;
Q is selected from

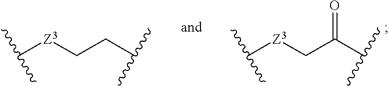

E is absent or selected from —N(R^{a})— and

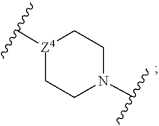

Z¹ and Z² are independently selected at each occurrence from 0 or N(R^{a});
Z³ is selected from CH₂, 0, N(R^{a}), and S;
Z⁴ is selected from CH and N;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 6-membered heterocyclyl)—(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, each of which may be optionally substituted with one or more groups selected from Y as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments of Formula IV, R¹⁰ is R^{10a}.

In some embodiments of Formula IV, R¹⁰ is NH—R^{10a}.

In some embodiments of Formula IV, R^{10a} can include a substituted aryl or a substituted or unsubstituted heteroaryl. For example, R^{10a} can include a 5-, 6- and 7-membered aromatic ring. The ring can be a carbocyclic, heterocyclic, fused carbocyclic, fused heterocyclic, bicarbocyclic, or biheterocyclic ring system, which is optionally substituted as described herein. In some embodiments when R^{10a} is an heteroaryl, R^{10a} can include a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₈) alkyl, phenyl or benzyl. Examples of aryl and heteroaryl rings include, but are not limited to, benzene, pyrrole, furan, thiophene, imidazole, oxazole, thiazole, triazole, pyrazole, pyridine, pyrazine, pyridazine and pyrimidine. The aromatic ring can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. In any of the above disclosed embodiments, R^{10a} can be optionally substituted with B-Q-L²-Sp-L¹-E-.

In some embodiments of Formula IV, R^{10a} includes a polycyclic aryl or heteroaryl ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic. For example, in some embodiments when R^{10a} is a heteroaryl, R^{10a} can include an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like. In any of the above disclosed embodiments, R^{10a} can be substituted optionally with B-Q-L²-Sp-Li-E-.

In specific examples of Formula IV, R^{10a} is selected from a substituted C₅-C₆ aryl or a substituted or unsubstituted C₂-C₉ heteroaryl. For example, R^{10a} can be selected from a substituted phenyl, substituted or unsubstituted furyl, substituted or unsubstituted imidazolyl, substituted or unsubstituted triazolyl, substituted or unsubstituted triazinyl, substituted or unsubstituted oxazoyl, substituted or unsubstituted isoxazoyl, substituted or unsubstituted thiazolyl, substituted or unsubstituted isothiazoyl, substituted or unsubstituted pyrazolyl, substituted or unsubstituted pyrrolyl, substituted or unsubstituted pyrazinyl, substituted or unsubstituted tetrazolyl, substituted or unsubstituted pyridyl, substituted or unsubstituted thienyl, substituted or unsubstituted pyrimidinyl, substituted or unsubstituted indolyl, substituted or unsubstituted isoquinolyl, substituted or unsubstituted quinolyl, substituted or unsubstituted benzothienyl, substituted or unsubstituted benzofuranyl, substituted or unsubstituted benzoxazolyl, substituted or unsubstituted benzimidazoly, substituted or unsubstituted benzothiazolyl, substituted or unsubstituted isoindolyl, substituted or unsubstituted indolinyl, substituted or unsubstituted isoindolinyl, substituted or unsubstituted substituted or unsubstituted quinoxalinyl, substituted or unsubstituted quinazolinyl, substituted or unsubstituted cinnolinyl, substituted or unsubstituted [2,3-c] or [3,2-c]-thienopyridyl, and the like. In some examples, R^{10a} is a substituted or unsubstituted fused C₄-C₉ heteroaryl, preferably unsubstituted indolyl. In any of the above disclosed embodiments, R^{10a} can be optionally substituted with B-Q-L²-Sp-Li-E-.

In some embodiments of Formula IV, R^{10a} may be an unsubstituted or substituted heterocycloalkyl. Examples of heterocycloalkyls include, but are not limited to, saturated 4-to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Further examples of heterocycloalkyls include, but are not limited to, dihydrothienyl, dihydropyranyl, dihydrofuryl, and dihydrothiazolyl. Even further examples of heterocycloalkyls include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. In any of the above disclosed embodiments, R^{10a} can be optionally substituted with B-Q-L²-Sp-Li-E-.

In some embodiments of Formula IV, R¹⁰ is

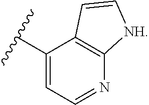 [SMILES: *c1ccnc2[nH]ccc12]

In some embodiments of Formula IV, R¹⁰ is

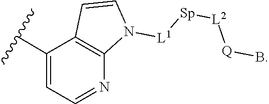

In some embodiments of Formula IV, R¹⁰ is

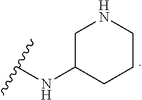 [SMILES: *NC1CCCNC1]

In some embodiments of Formula IV, R¹⁰ is Formula IV, R¹⁰ is

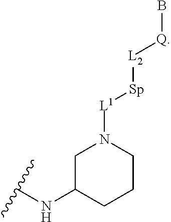

In some embodiments of Formula IV, R¹⁰ is

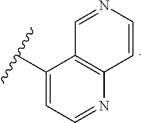 [SMILES: *c1ccnc2ccncc12]

In some embodiments of Formula IV, R¹⁰ is

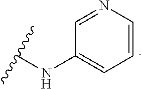 [SMILES: *Nc1cccnc1]

In some embodiments of Formula IV, R¹⁰ is

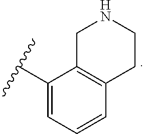 [SMILES: *c1cccc2c1CNCC2]

In some embodiments of Formula IV, R¹⁰ is

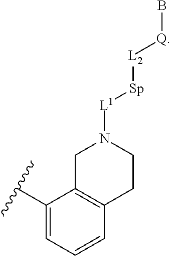

In some embodiments of Formula IV, R^{11a} and R^{11b} may be brought together with the carbon to which they are attached to form a, unsubstituted or substituted cycloalkyl ring. For example, R^{11a} and R^{11b} may be brought together with the carbon to which they are attached to form a cyclopropyl ring, a cyclobutyl ring, a cyclopentyl ring, or a cyclohexyl ring. In particular embodiments, R^{11a} and R^{11b} may be brought together with the carbon to which they are attached to form a cyclopropyl ring or a cyclobutyl ring. In particular examples, R^{11a} and R^{11b} may be brought together with the carbon to which they are attached to form a cyclopropyl ring. The cycloalkyl ring can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN.

In some embodiments of Formula IV, R^{11a} and R^{11b} may be brought together with the carbon to which they are attached to form an unsubstituted or substituted heterocycloalkyl ring containing at least one ring heteroatom (for example, 1 or 2 ring heteroatoms) selected from —O—, —N(Y¹)—, and S, wherein Y¹ is hydrogen, C₁-C₆ alkyl, cycloalkyl, aryl, or heteroaryl. In particular embodiments, R^{11a} and R^{11b} may be brought together with the carbon to which they are attached to form a heterocycloalkyl ring containing 1 or 2 ring heteroatoms selected from —O— and —N(Y¹)—. In particular embodiments, R^{11a} and R^{11b} may be brought together with the carbon atom to which they are attached to form a 3- to 6-membered heterocycloalkyl ring containing 1 or 2 ring oxygen atoms. In particular embodiments, R^{11a} and R^{11b} may be brought together with the carbon to which they are attached to form a 3- to 6-membered heterocycloalkyl ring containing 1 or 2 ring nitrogen atoms. The heterocycloalkyl ring can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN.

In some embodiments of Formula IV, R^{11a} and R^{11b} are brought together with the carbon to which they are attached to form

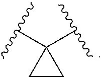 [SMILES: *C1(*)CC1]

In some embodiments of Formula IV, R^{11a} and R^{11b} are brought together with the carbon to which they are attached to form

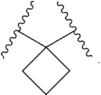 [SMILES: *C1(*)CCC1]

In some embodiments of Formula II, R^{31a} and R^{31b} are brought together with the carbon to which they are attached to form

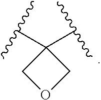 [SMILES: *C1(*)COC1]

In some embodiments of Formula IV, R^{11a} and R^{11b} are brought together with the carbon to which they are attached to form

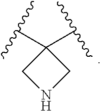 [SMILES: *C1(*)CNC1]

In some embodiments of Formula IV, R¹² is R^{12a}.

In some embodiments of Formula IV, R¹² is —NH—R^{12b}.

In some embodiments of Formula IV, R^{12a} is C₁-C₆ alkyl. In some embodiments of Formula II, R^{12a} is selected from methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In particular embodiments of Formula II, R^{12a} is methyl. In any of the above embodiments, R^{12a} can be optionally substituted with B-Q-L²-Sp-L¹-E-.

In some embodiments of Formula IV, R^{12a} is —(C₁-C₆ alkyl)-(substituted or unsubstituted aryl). In particular embodiments of Formula IV, R^{12a} is —CH₂-(substituted or unsubstituted aryl). The aryl in R^{12a} can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. In particular embodiments, R^{12a} is —CH₂-(aryl substituted with amino). In particular embodiments, R^{12a} is benzyl. In any of the above embodiments, R^{12a} can be optionally substituted with B-Q-L²-Sp-L¹-E-.

In some embodiments of Formula IV, R^{12b} is hydrogen.

In some embodiments of Formula IV, R^{12b} is —(C₁-C₆ alkyl)-(substituted or unsubstituted aryl). In particular embodiments of Formula IV, R^{12b} is —CH₂-(substituted or unsubstituted aryl). The aryl in R^{32b} can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. In particular embodiments, R^{12b} is —CH₂-(aryl substituted with amino). In particular embodiments, R^{12b} is benzyl. In any of the above embodiments, R^{12b} can be optionally substituted with B-Q-L²-Sp-L¹-E-.

In some embodiments of Formula IV, R¹² is

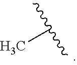

In some embodiments of Formula IV, R¹² is

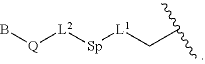

In some embodiments of Formula II, R¹² is

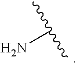 [SMILES: *N]

In some embodiments of Formula IV, R¹² is

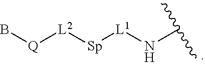

In some embodiments of Formula II, R¹² is

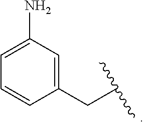 [SMILES: *Cc1cccc(N)c1]

In some embodiments of Formula II, R¹² is

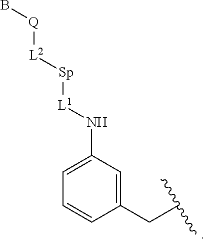

In some embodiments of Formula II, R¹² is

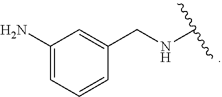 [SMILES: *NCc1cccc(N)c1]

In some embodiments of Formula II, R¹² is

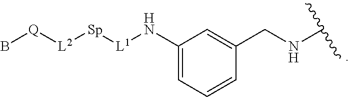

In some embodiments of Formula II, Formula III, or Formula IV, E is a bond. In some embodiments of Formula II, Formula III, or Formula IV, E is —N(R^{a})—. In some embodiments of Formula II, Formula III, or Formula IV, E is —NH—. In some embodiments of Formula II, Formula III, or Formula IV, E is

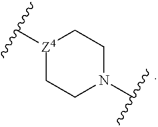

In some embodiments of Formula II, Formula III, or Formula IV, E is

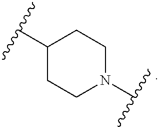 [SMILES: *C1CCN(*)CC1]

In some embodiments of Formula II, Formula III, or Formula IV, E is

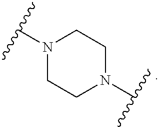 [SMILES: *N1CCN(*)CC1]

In some embodiments of Formula I, Formula II, Formula III or Formula IV, L¹ is C₁-C₁₀ alkyl. In some embodiments of Formula I, Formula II, Formula III or Formula IV, L¹ is selected from

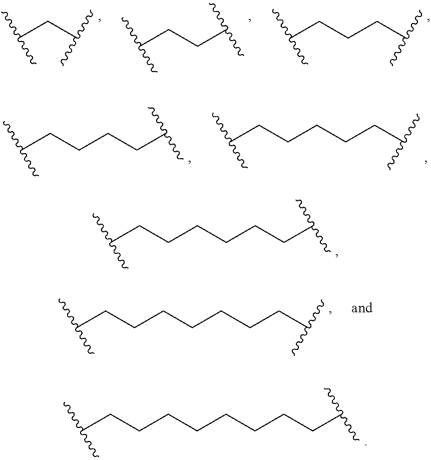

In some embodiments of Formula I, Formula II, Formula III or Formula IV, L¹ is

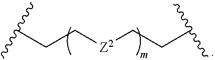

In some embodiments of Formula I, Formula II, Formula III or Formula IV, L¹ is selected from

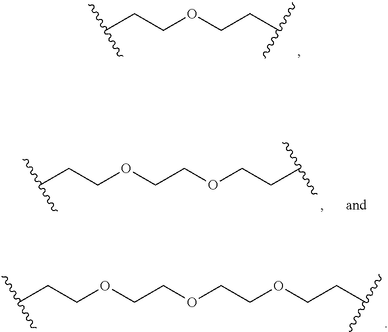 [SMILES: *CCOCC* ; *CCOCCOCC* ; *CCOCCOCCOCC*]

In some embodiments of Formula I, Formula II, Formula III or Formula IV, L² is C₁-C₁₀ alkyl. In some embodiments of Formula I, Formula II, or Formula III, L² is selected from

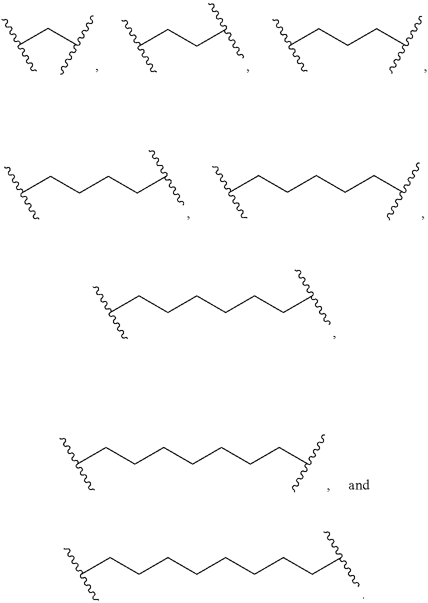

In some embodiments of Formula I, Formula II, Formula III or Formula IV, L² is

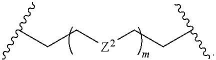

In some embodiments of Formula I, Formula II, Formula III or Formula IV, L² is selected from

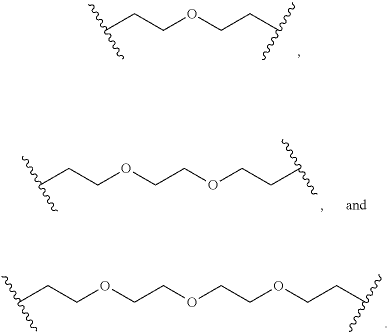 [SMILES: *CCOCC* ; *CCOCCOCC* ; *CCOCCOCCOCC*]

In some embodiments of Formula I, Formula II, Formula III or Formula IV, Sp is 5-to 6-membered heteroaryl. In some embodiments of Formula I, Formula II, Formula III or Formula IV, Sp is

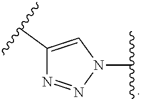 [SMILES: *c1cn(*)nn1]

In some embodiments of Formula I, Formula II, or Formula III, Sp is 3- to 6-membered heterocyclyl. In some embodiments of Formula I, Formula II, Formula III or Formula IV, Sp is

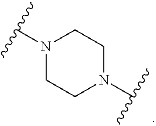 [SMILES: *N1CCN(*)CC1]

In some embodiments of Formula I, Formula II, Formula III or Formula IV, S is

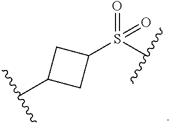 [SMILES: *C1CC(S(*)(=O)=O)C1]

In some embodiments of Formula I, Formula II, Formula III or Formula IV, Sp is

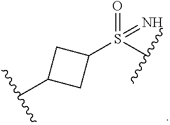 [SMILES: *C1CC(S(*)(=N)=O)C1]

In some embodiments of Formula I, Formula II, Formula III or Formula IV, Sp is

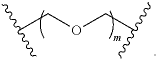

In some embodiments of Formula I, Formula II, Formula III or Formula IV, Sp is

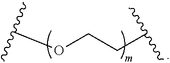

In some embodiments of Formula I, Formula II, Formula III or Formula IV, Q is a bond.

In some embodiments of Formula I, Formula II, Formula III or Formula IV, Q is

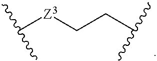

In some embodiments of Formula I, Formula II, Formula III or Formula IV, Q is

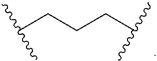 [SMILES: *CCC*]

In some embodiments of Formula I, Formula II, and Formula III or Formula IV Q is

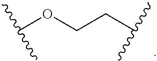 [SMILES: *CCO*]

In some embodiments of Formula I, Formula II, Formula III or Formula IV, Q is

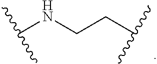 [SMILES: *CCN*]

In some embodiments of Formula I, Formula II, Formula III or Formula IV, Q is

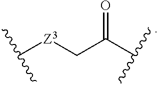

In some embodiments of Formula I, Formula II, Formula III or Formula IV, Q is

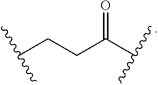 [SMILES: *CCC(*)=O]

In some embodiments of Formula I, Formula II, Formula III or Formula IV, Q is

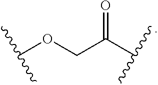 [SMILES: *OCC(*)=O]

In some embodiments of Formula I, Formula II, Formula III or Formula IV, Q is

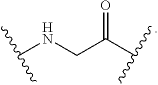 [SMILES: *NCC(*)=O]

The E3 ubiquitin ligase-recruiting moiety B is a chemical moiety capable of recruiting an E3 ubiquitin ligase to a given substrate protein (for example, TAF1) resulting in its targeted degradation. In some embodiments, B is a chemical moiety based upon a high affinity small molecule for E3 ubiquitin ligases, such as von Hippel-Lindau or cereblon. In some embodiments, B is a chemical moiety based upon von Hippel-Lindau binder such as VH032 or VH298. In some embodiments, B is a chemical moiety based upon a cereblon binder such as thalidomide, lenalidomide, or pomalidomide.

In some embodiments of Formula I, Formula II, Formula III or Formula IV, B is selected from

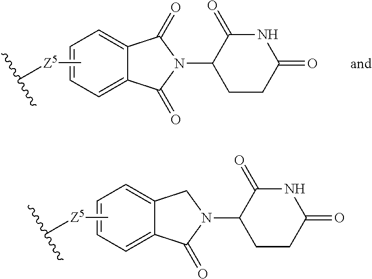

wherein Z⁵ is selected from O, N(R^{a}), and CH₂.

In some embodiments of Formula I, Formula II, Formula III or Formula IV, B is selected from

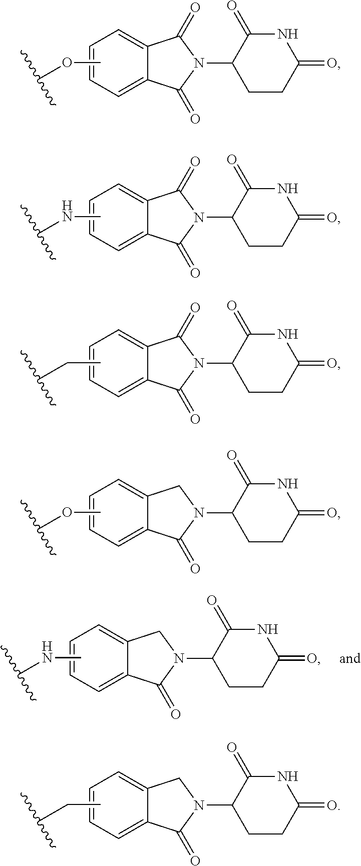

In some embodiments of Formula I, Formula II, Formula III or Formula IV, B is selected from

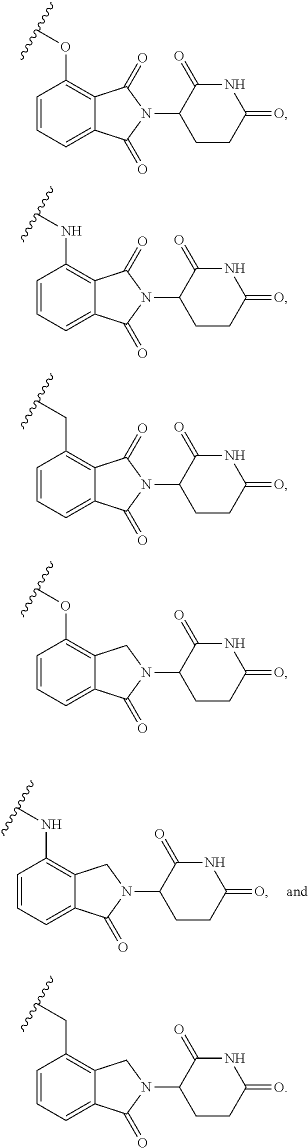

In some embodiments of Formula I, Formula II, Formula III or Formula IV, B is selected from

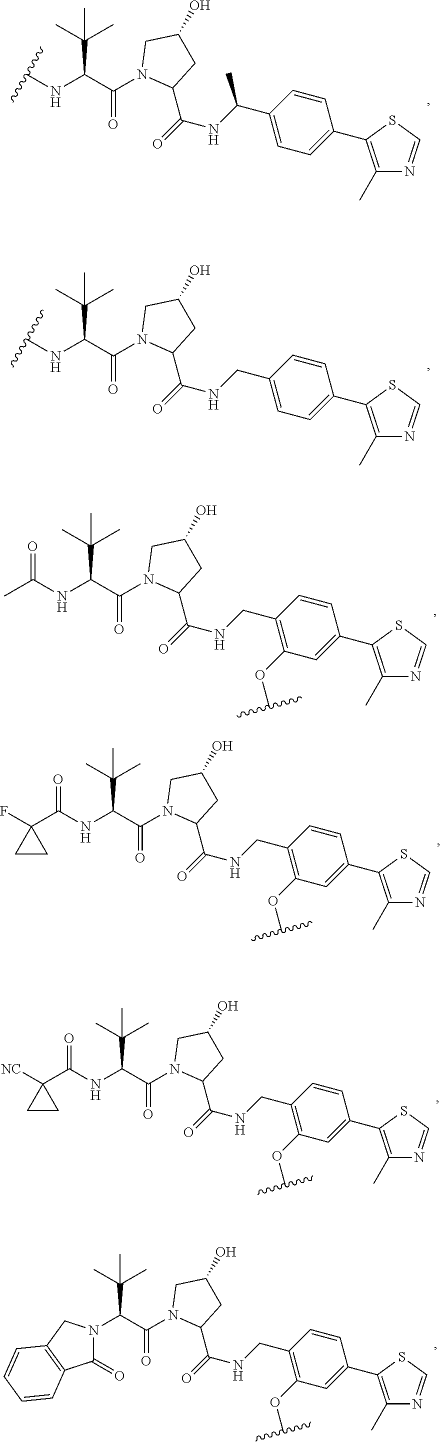 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

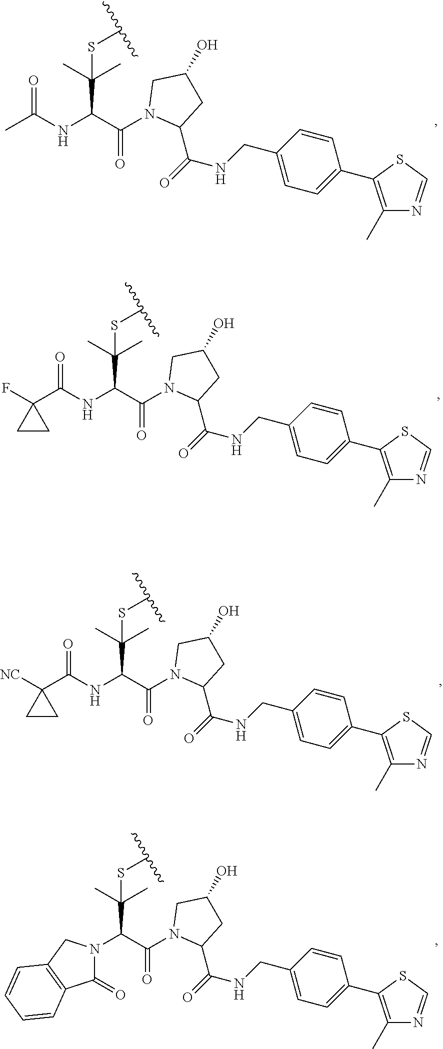 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O]

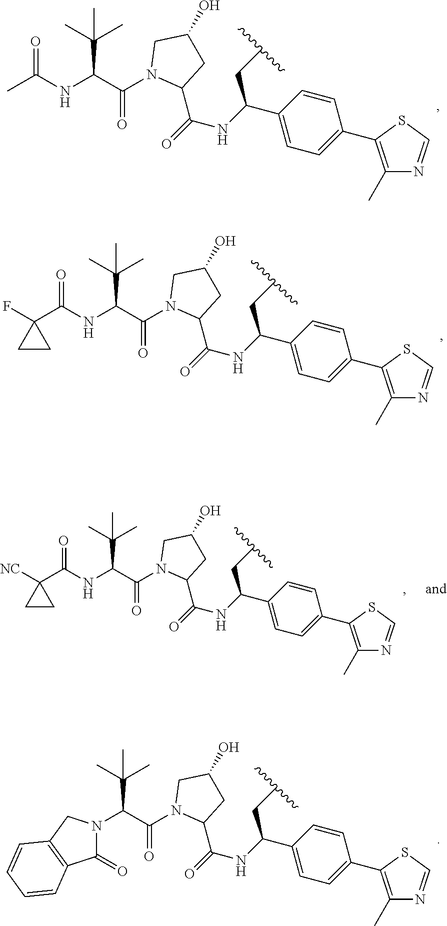 [SMILES: *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)C)c1ccc(-c2scnc2C)cc1 ; *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

Representative compounds of the present disclosure include, but are not limited to:

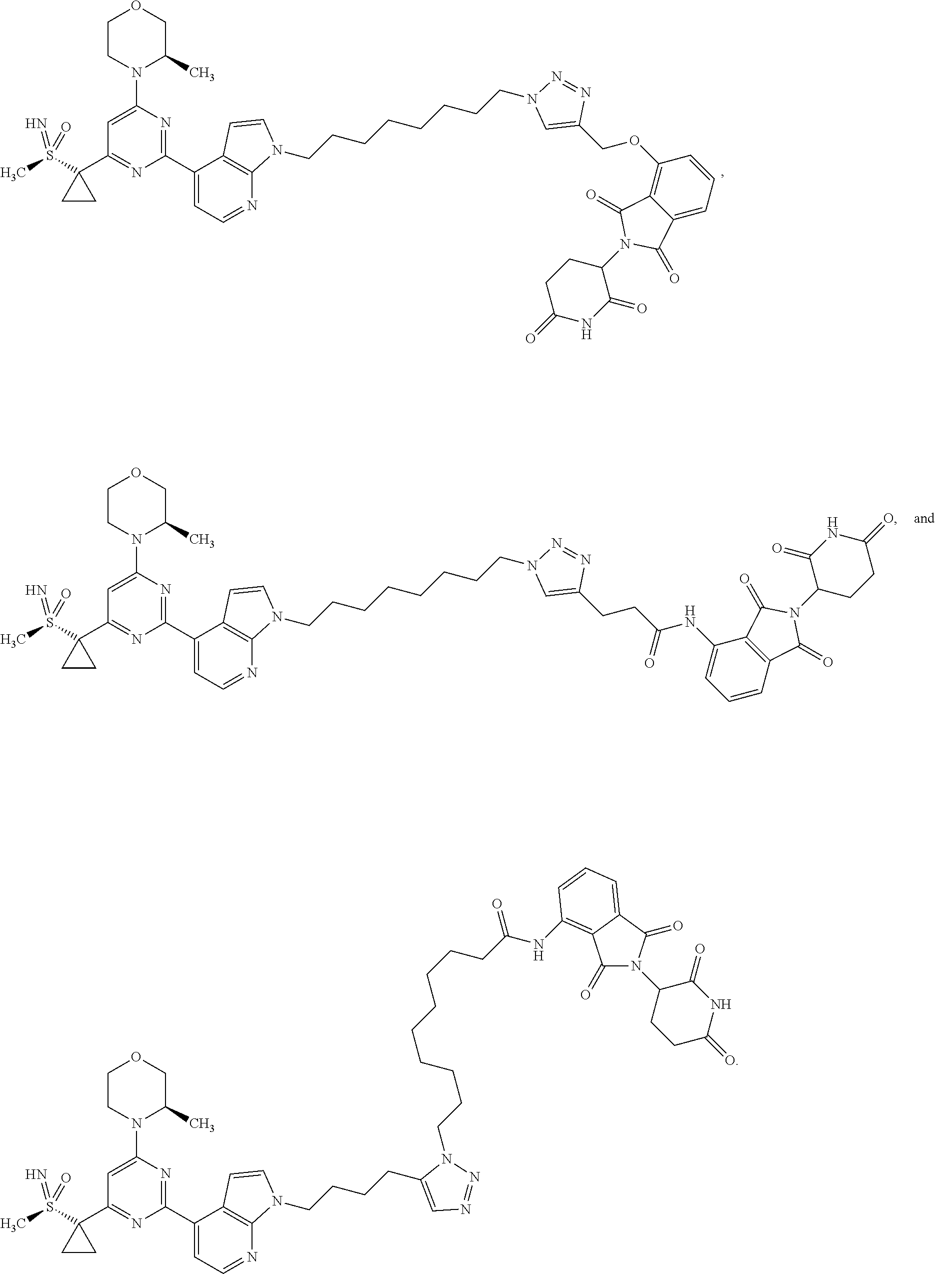 [SMILES: C[C@@H]1COCCN1c1cc(C2([S@](C)(=N)=O)CC2)nc(-c2ccnc3c2ccn3CCCCCCCCn2cc(COc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCCCCCn2cc(CCC(=O)Nc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCc2cnnn2CCCCCCCCCC(=O)Nc2cccc3c2C(=O)N(C2CCC(=O)NC2=O)C3=O)n1]

The present disclosure also includes compounds of Formula I, Formula II, or Formula III with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P ³²P, ³⁵S, ³⁶Cl, and 125I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen- free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard- filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard- filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some embodiments, the compounds can facilitate degradation of TAF1. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma,hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

In another aspect, the following particular embodiments of the disclosure are also provided:

Embodiment 1. A compound of Formula I, Formula II, or Formula III:

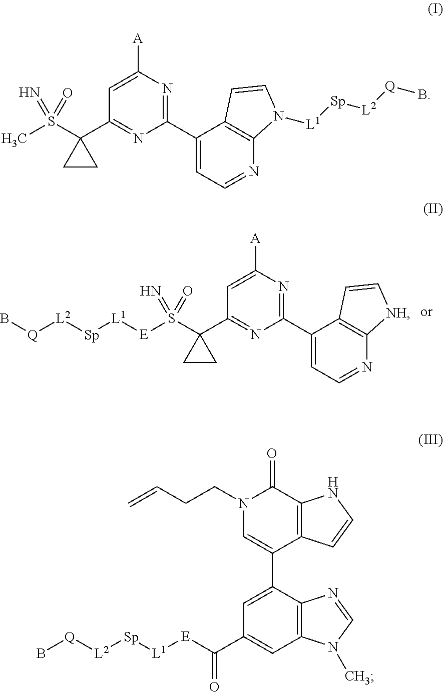

or a pharmaceutically acceptable salt thereof; wherein:
A is selected from:

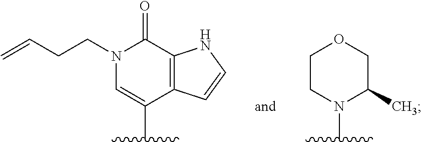 [SMILES: *c1cn(CCC=C)c(=O)c2[nH]ccc12 ; "and" ; *N1CCOC[C@H]1C]

B is an E3 ubiquitin ligase-recruiting moiety;
L¹ and L² are independently selected from C₁-C₁₀ alkyl, C₂-C₁₀ alkenyl, C₂-C₁₀ alkynyl, and

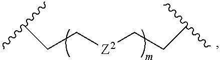

each of which may be substituted with one or more groups independently selected from X as allowed by valency;

Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl,

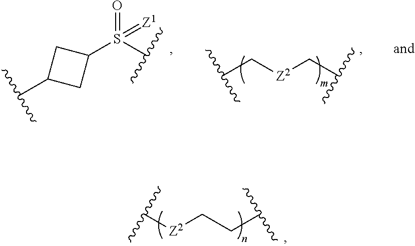

each of which may be optionally substituted with one of more groups independently selected from X as allowed by valency;
m is independently selected at each occurrence from 1, 2, 3, 4, 5, 6, 7, 8, 9, or 10; Q is a bond or selected from and

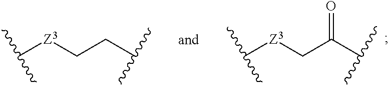

E is a bond or selected from —N(R^{a})— and;

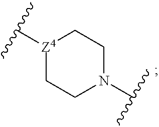

Z¹ and Z² are independently selected at each occurrence from O or N(R^{a});
Z³ is selected from CH₂, O, N(R^{a}), and S;
Z⁴ is selected from CH and N;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 6-membered heterocyclyl)—(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, each of which may be optionally substituted with one or more groups selected from Y as allowed by valency;
R^{Z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.
   Embodiment 2. The compound of embodiment 1, wherein the compound is of Formula I:

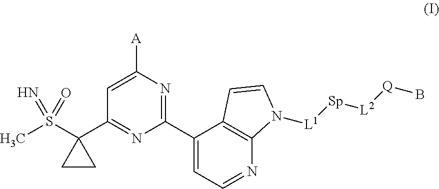

or a pharmaceutically acceptable salt thereof.
   Embodiment 3. The compound of embodiment 1, wherein the compound is of Formula II:

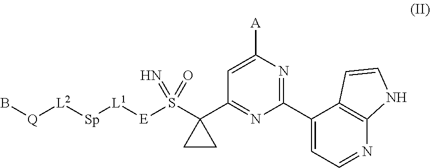

or a pharmaceutically acceptable salt hereof.
   Embodiment 4. The compound of any one of embodiments 1-3, wherein A is

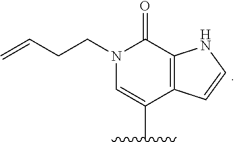 [SMILES: *c1cn(CCC=C)c(=O)c2[nH]ccc12]

Embodiment 5. The compound of any one of embodiments 1-3, wherein A is

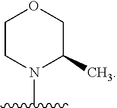 [SMILES: *N1CCOC[C@H]1C]

Embodiment 6. The compound of embodiment 1, wherein the compound is of Formula III:

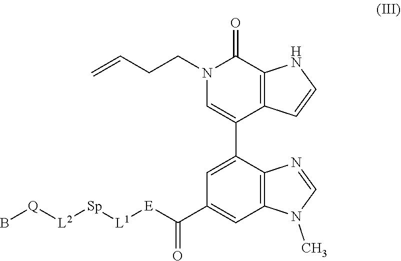

or a pharmaceutically acceptable salt thereof.
   Embodiment 7. A compound of Formula IV

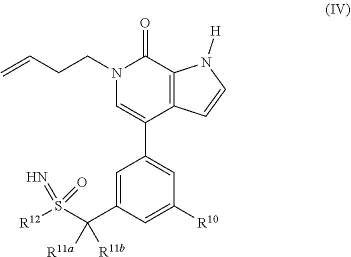

or a pharmaceutically acceptable salt thereof;
wherein:
R¹⁰ is selected from R^{10a} and —NH—R^{10a};
R^{10a} is selected from aryl, heteroaryl, cycloalkyl, or heterocycloalkyl, each of which may be optionally substituted with one or more groups independently selected from X as allowed by valency;
R^{11a} and R^{11b} are brought together with the carbon to which they are attached to form a cycloalkyl or heterocycloalkyl ring which may be optionally substituted with one or more groups independently selected from X as allowed by valency;
R¹² is selected from R^{12a} and —NH—R^{12b};
R^{12a} is selected from C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;
R^{12b} is selected from hydrogen C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;
wherein at least one of R¹⁰ or R¹² is substituted with B-Q-L²-Sp-Li-E-;
B is an E3 ubiquitin ligase-recruiting moiety;
L¹ and L² are independently selected from C₁-C₁₀ alkyl, C₂-C₁₀ alkenyl, C₂-C₁₀ alkynyl, and

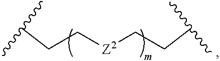

each of which may be substituted with one or more groups independently selected from X as allowed by valency;

Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl,

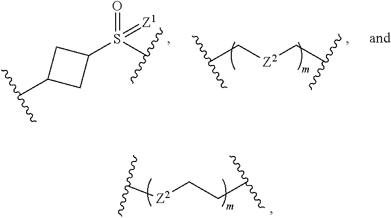

each of which may be optionally substituted with one or more groups independently selected from X as allowed by valency;
m is independently selected at each occurrence from 1, 2, 3, 4, 5, 6, 7, 8, 9, or 10;
Q is selected from

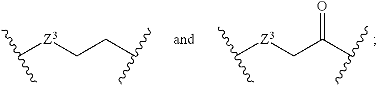

E is absent or selected from —N(R^{a})— and;

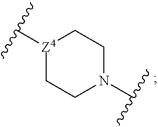

Z¹ and Z² are independently selected at each occurrence from O or N(R^{a});
Z³ is selected from CH₂, O, N(R^{a}), and S;
Z⁴ is selected from CH and N;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 6-membered heterocyclyl)—(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, each of which may be optionally substituted with one or more groups selected from Y as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)-(C₀-C₃ alkyl)-, (5- to 10-membered monocyclic or bicyclic heteroaryl)—(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.
   Embodiment 8. The compound of embodiment 7, wherein R¹⁰ is R^{10a}.
   Embodiment 9. The compound of embodiment 7, wherein R¹⁰ is —NH—R^{10a}.
   Embodiment 10. The compound of any one of embodiments 7-9, wherein R^{10a} is unsubstituted or substituted aryl.
   Embodiment 11. The compound of any one of embodiments 7-9, wherein R^{10a} is unsubstituted or substituted heteroaryl.
   Embodiment 12. The compound of any one of embodiments 7-9, wherein R^{10a} is unsubstituted or substituted heterocycloalkyl.
   Embodiment 13. The compound of embodiment 7, wherein R¹⁰ is selected from:

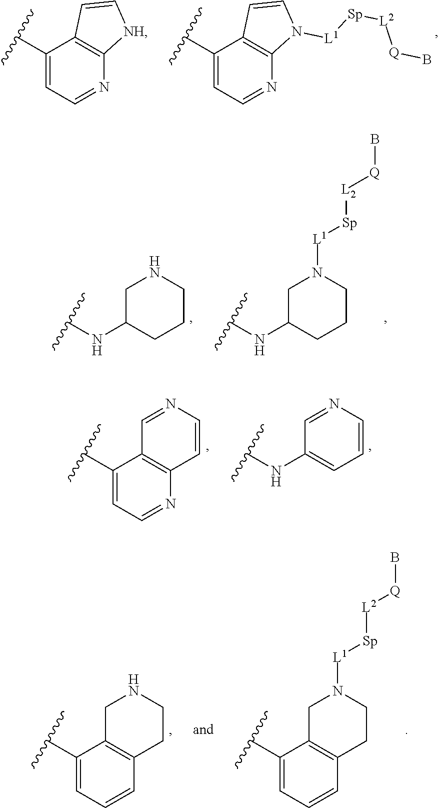

Embodiment 14. The compound of any one of embodiments 7-13, wherein R^{11a} and R^{11b} are brought together with the carbon to which they are attached to form a substituted or unsubstituted cycloalkyl ring.
Embodiment 15. The compound of any one of embodiments 7-13, wherein R^{11a} and R^{11b} are brought together with the carbon to which they are attached to form a substituted or unsubstituted heterocycloalkyl ring.
Embodiment 16. The compound of any one of embodiments 7-13, wherein R^{11a} and R^{11b} are brought together with the carbon to which they are attached to form:

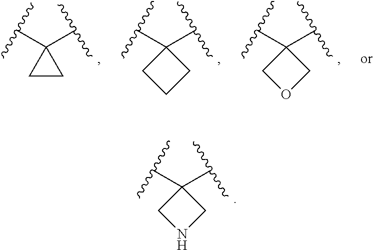 [SMILES: *C1(*)CC1 ; *C1(*)CCC1 ; *C1(*)COC1 ; *C1(*)CNC1]

Embodiment 17. The compound of any one of embodiments 7-16, wherein R¹² is R^{12a}.
Embodiment 18. The compound of embodiment 17, wherein R^{12a} is C₁-C₆ alkyl.
Embodiment 19. The compound of embodiment 17, wherein R^{12a} is methyl.
Embodiment 20. The compound of embodiment 17, wherein R^{12a} is —(C₁-C₆ alkyl)-(substituted or unsubstituted aryl).
Embodiment 21. The compound of any one of embodiments 7-13, wherein R¹² is —NH—R^{12b}.
Embodiment 22. The compound of embodiment 21, wherein R^{12b} is hydrogen.
Embodiment 23. The compound of embodiment 21, wherein R^{12b} is —(C₁-C₆ alkyl)-(substituted or unsubstituted aryl).

Embodiment 24. The compound of any one of embodiments 7-13, wherein R¹² is selected from:

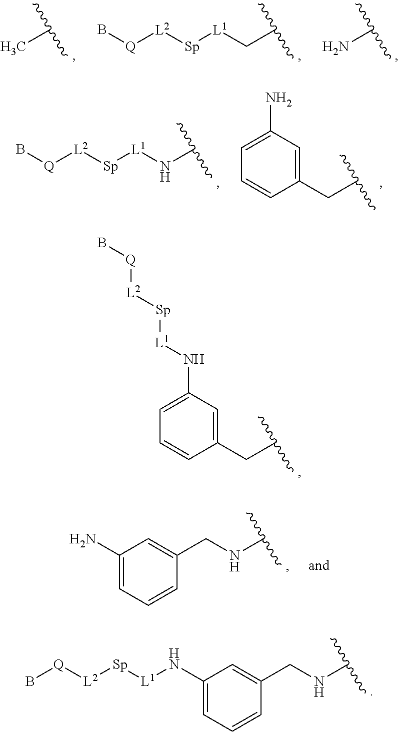

Embodiment 25. The compound of any one of embodiments 1 and 3-24, wherein E is a bond.
Embodiment 26. The compound of any one of embodiments 1 and 3-24, wherein E is —N(R^{a})—.
Embodiment 27. The compound of any one of embodiments 1 and 3-24, wherein E is

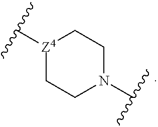

Embodiment 28. The compound of embodiment 27, wherein Z⁴ is CH.
Embodiment 29. The compound of embodiment 27, wherein Z⁴ is N.
Embodiment 30. The compound of any one of embodiments 1-29, wherein L¹ is C₁-C₁₀ alkyl.
Embodiment 31. The compound of any one of embodiments 1-30, wherein L² is C₁-C₁₀ alkyl.
Embodiment 32. The compound of any one of embodiments 1-31, wherein Sp is 5- to 6-membered heteroaryl.
Embodiment 33. The compound of embodiment 32, wherein Sp is

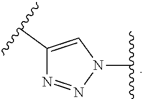 [SMILES: *c1cn(*)nn1]

Embodiment 34. The compound of any one of embodiments 1-31, wherein Sp is 3- to 6-membered heterocyclyl.

Embodiment 35. The compound of embodiment 34, wherein Sp is

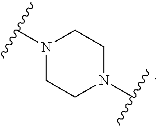 [SMILES: *N1CCN(*)CC1]

Embodiment 36. The compound of any one of embodiments 1-31, wherein Sp is selected from

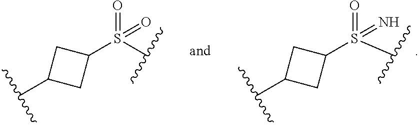 [SMILES: *C1CC(S(*)(=O)=O)C1 ; *C1CC(S(*)(=N)=O)C1]

Embodiment 37. The compound of any one of embodiments 1-31, wherein Sp is

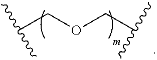

Embodiment 38. The compound of any one of embodiments 1-31, wherein Sp is

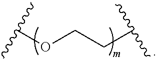

Embodiment 39. The compound of any one of embodiments 1-38, wherein Q is a bond.
Embodiment 40. The compound of any one of embodiments 1-38, wherein Q is

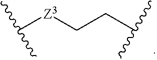

Embodiment 41. The compound of any one of embodiments 1-38, wherein Q is

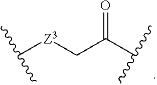

Embodiment 42. The compound of embodiment 40 or embodiment 41, wherein Z³ is selected from CH₂, O, and NH.
Embodiment 43. The compound of any one of embodiments 1-42, wherein B is selected from

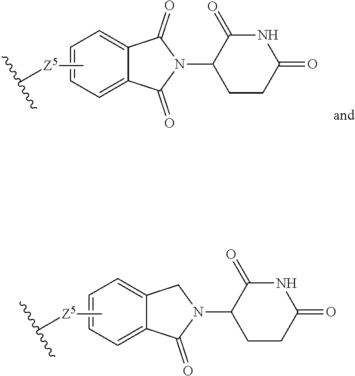

wherein Z⁵ is selected from O, N(R^{a}), and CH₂.
Embodiment 44. The compound of any one of embodiments 1-42, wherein B is selected from

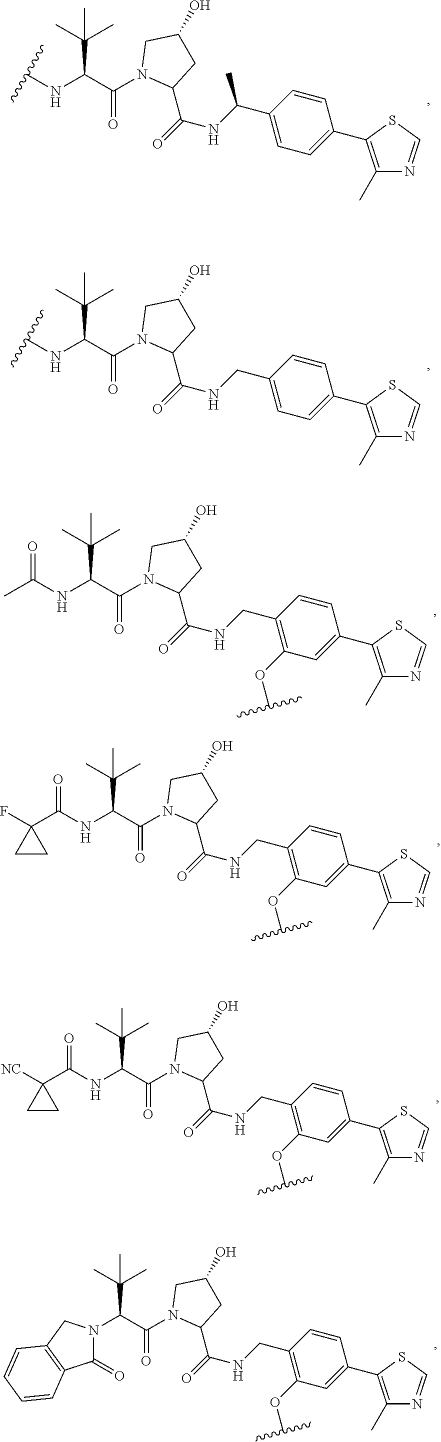 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

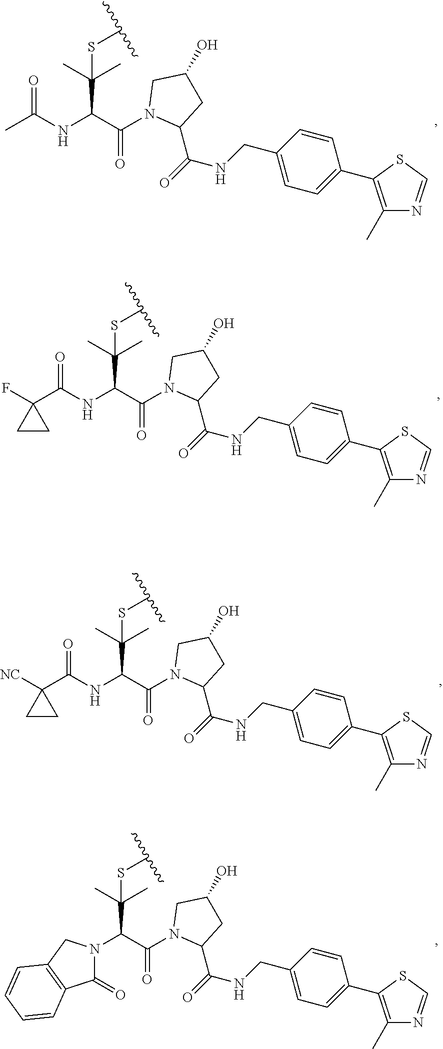 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O]

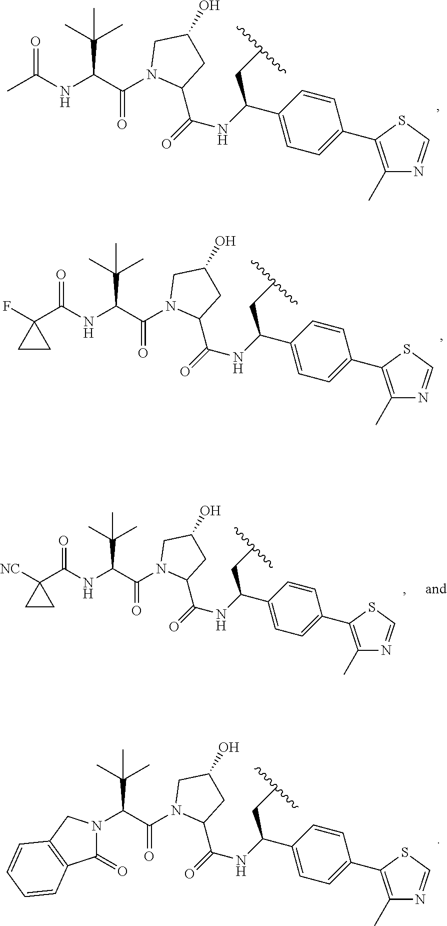 [SMILES: *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)C)c1ccc(-c2scnc2C)cc1 ; *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

Embodiment 45. The compound of embodiment 1, wherein the compound is selected from:

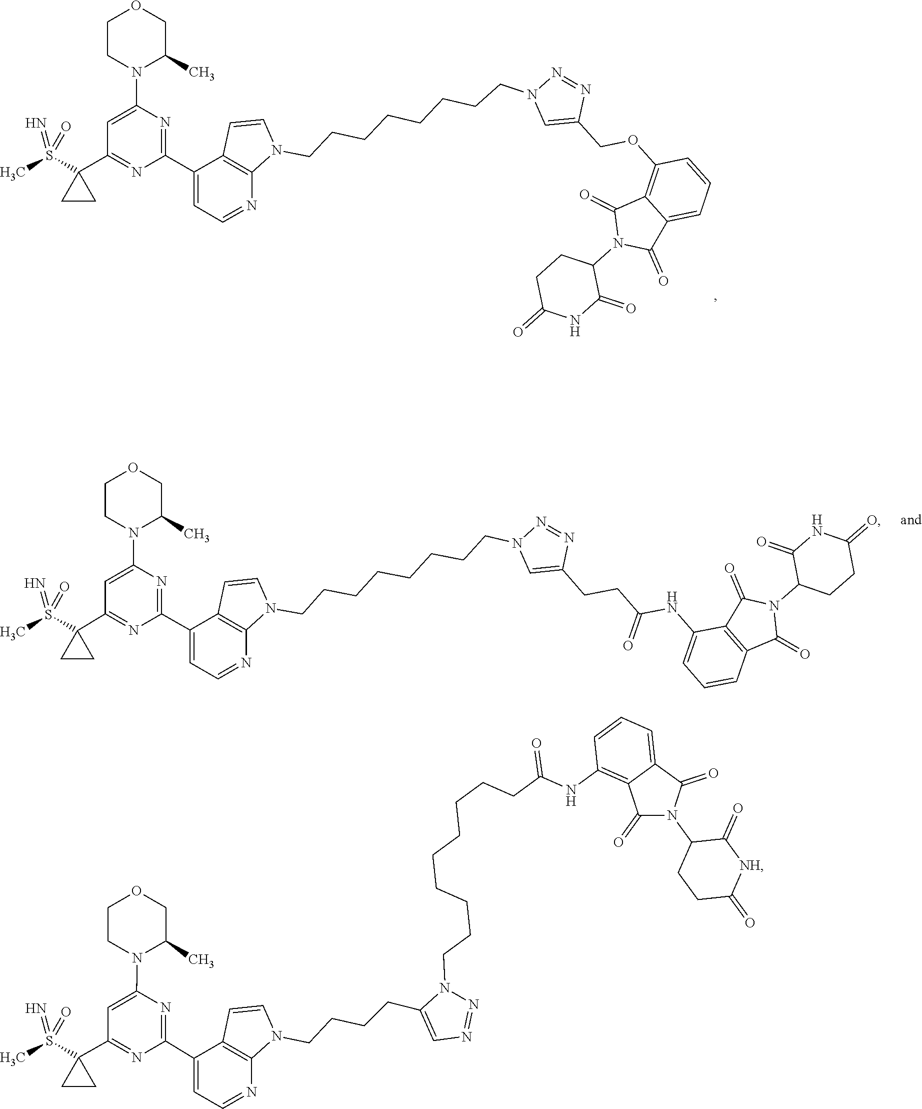 [SMILES: C[C@@H]1COCCN1c1cc(C2([S@](C)(=N)=O)CC2)nc(-c2ccnc3c2ccn3CCCCCCCCn2cc(COc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCCCCCn2cc(CCC(=O)Nc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCc2cnnn2CCCCCCCCCC(=O)Nc2cccc3c2C(=O)N(C2CCC(=O)NC2=O)C3=O)n1]

or a pharmaceutically acceptable salt thereof.
Embodiment 46. The compound of embodiment 1, wherein the compound is selected from:

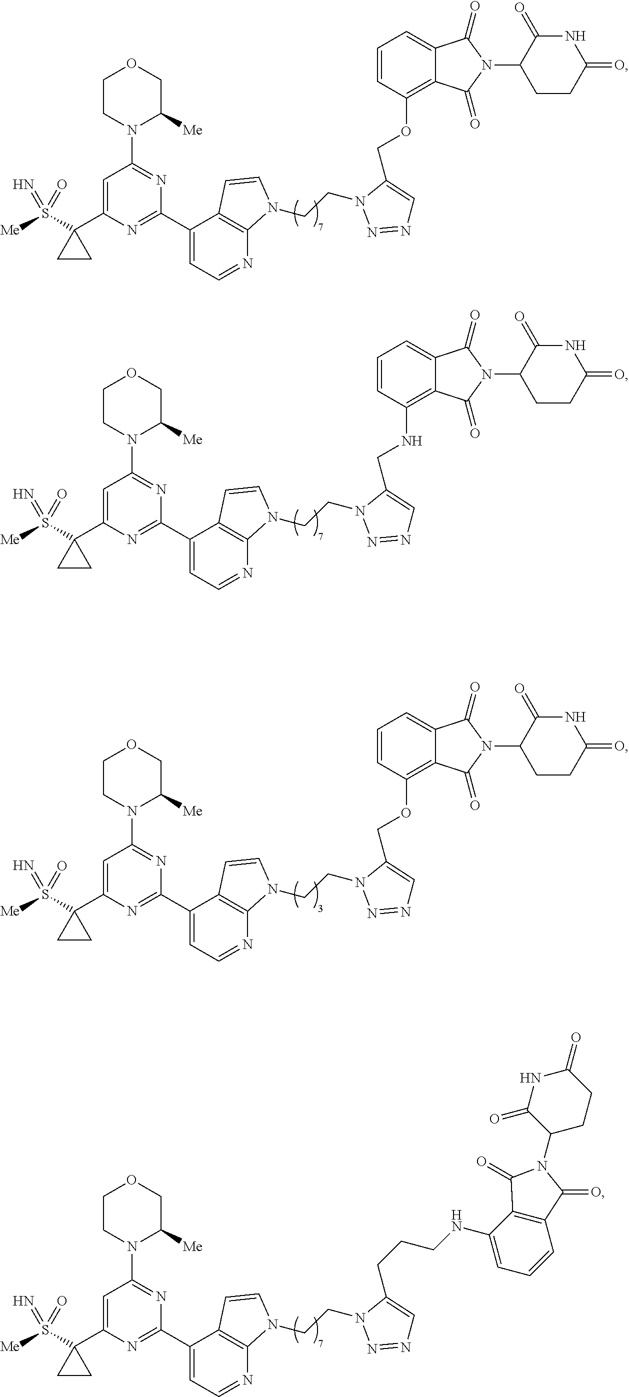

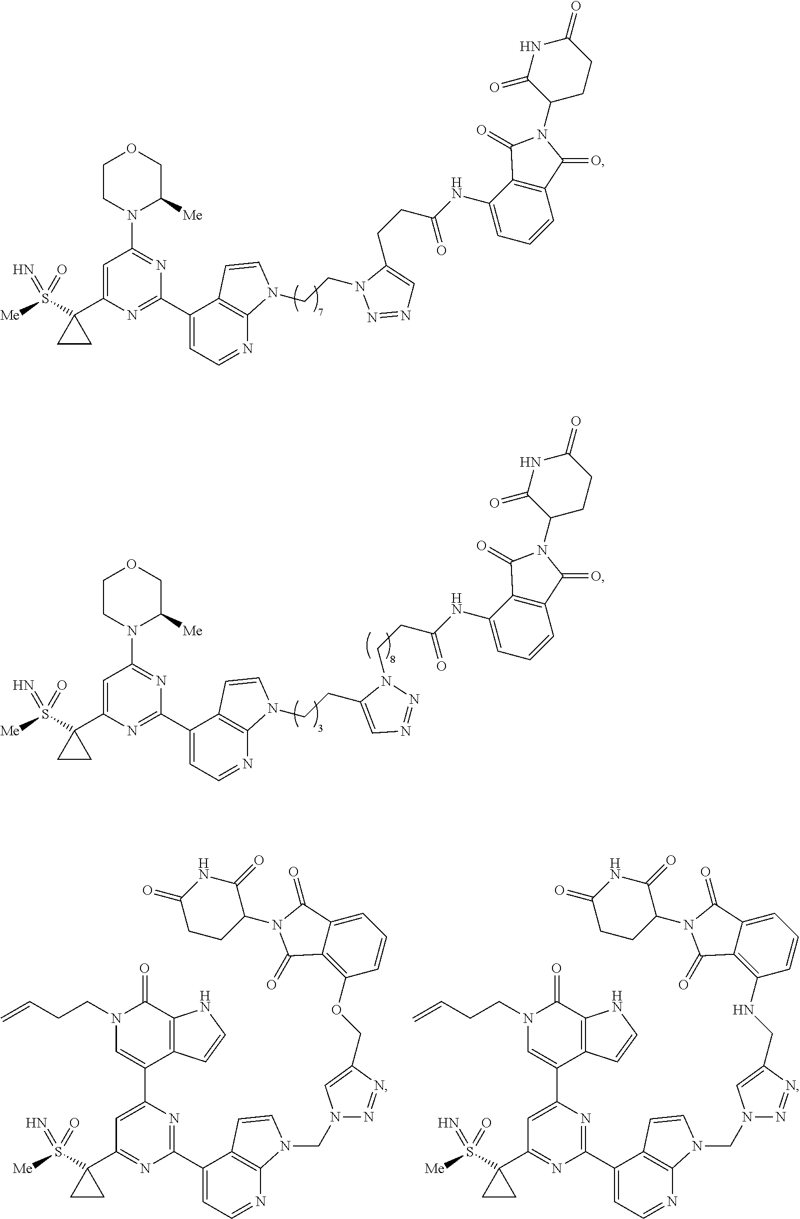

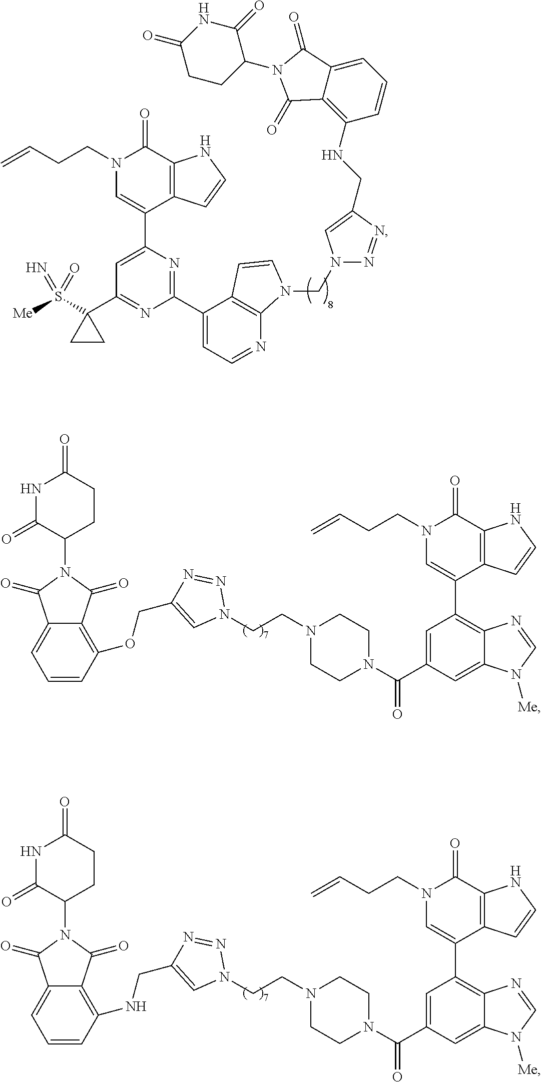

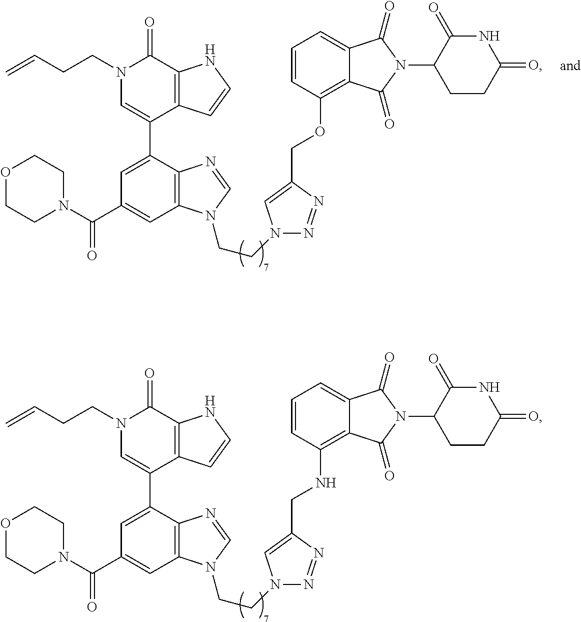

or a pharmaceutically acceptable salt thereof.
Embodiment 47. A pharmaceutical composition comprising a compound of any one of embodiments 1-46, or a pharmaceutically acceptable salt thereof, and a pharmaceutically acceptable carrier.
Embodiment 48. A method of treating cancer in a subject in need thereof comprising administering to the subject a therapeutically effective amount of a compound of any one of embodiments 1-46, or a pharmaceutically acceptable salt thereof, or a pharmaceutical composition of embodiment 47.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Synthesis of Compounds

Representative compounds of the present disclosure may be prepared as described in the following schemes:

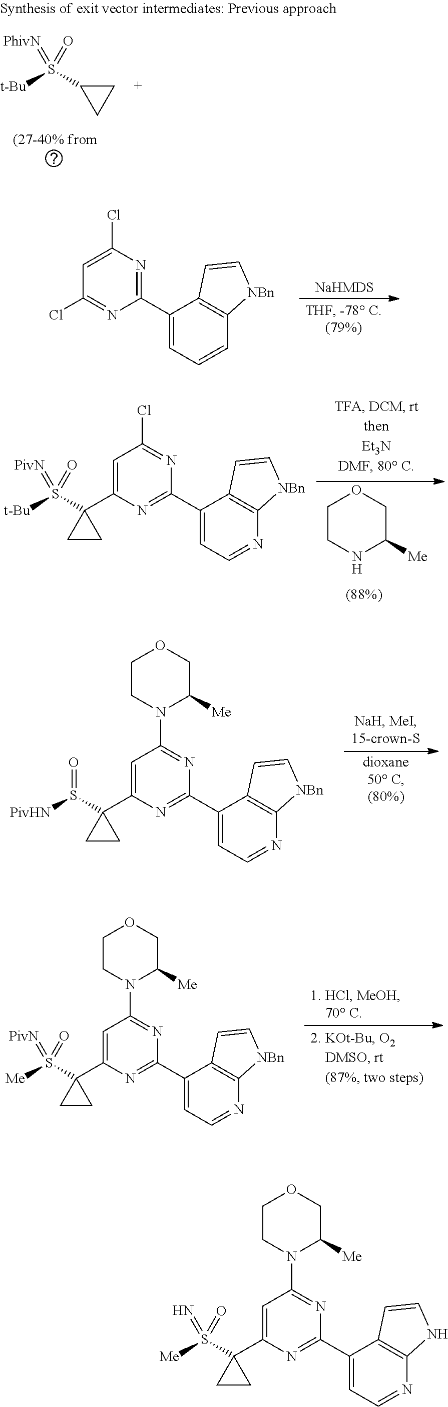

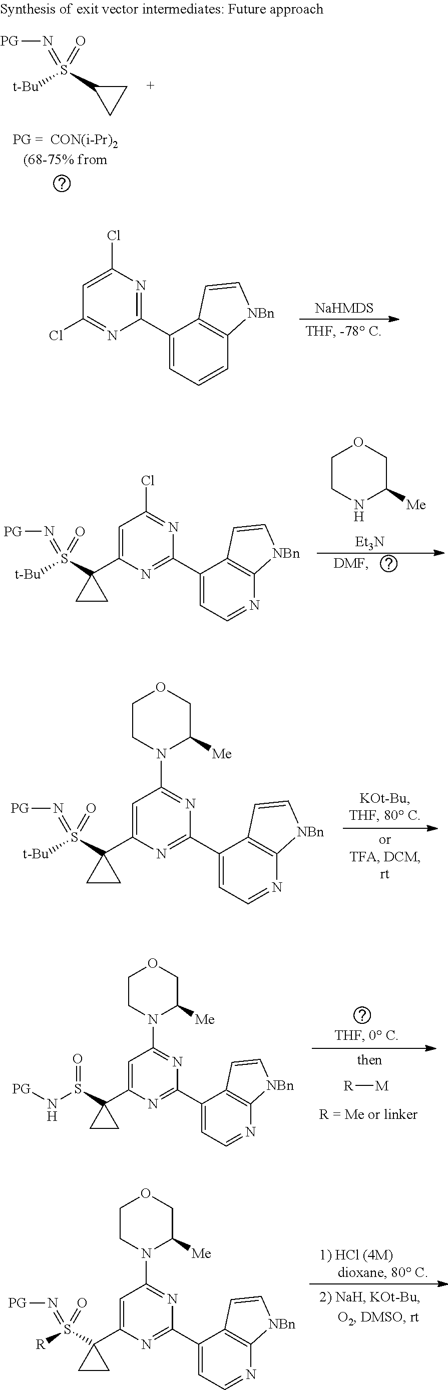

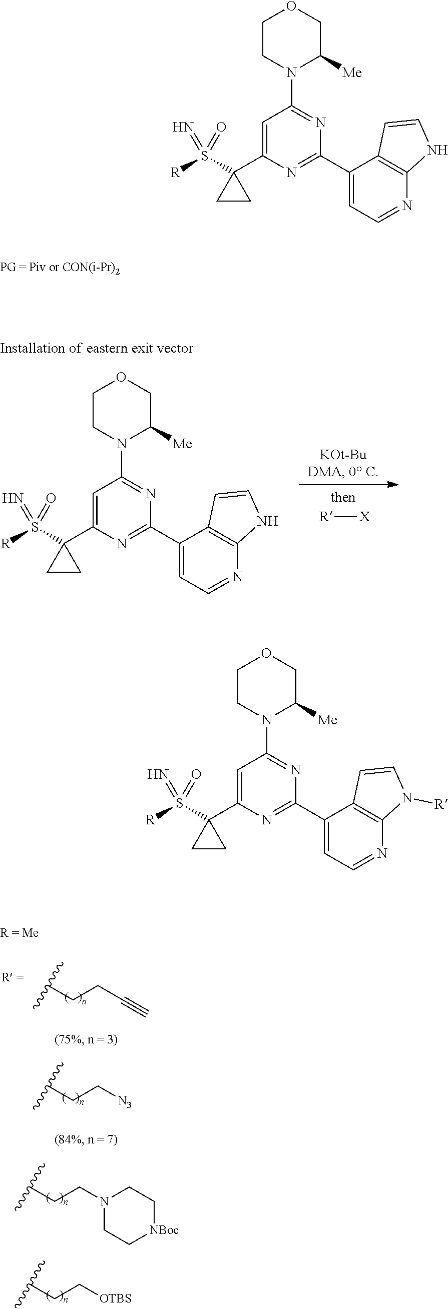

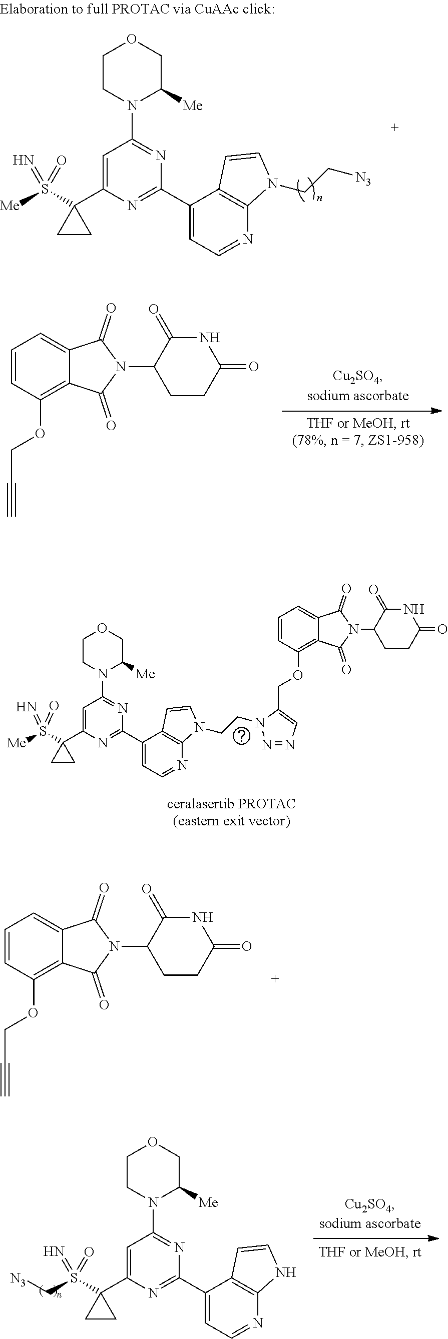

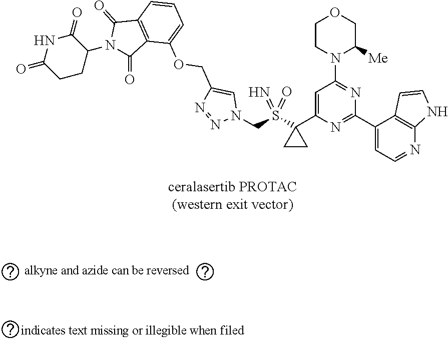 [SMILES: C[C@@H]1COCCN1c1cc(C2([S@](=N)(=O)Cn3cc(COc4cccc5c4C(=O)N(C4CCC(=O)NC4=O)C5=O)nn3)CC2)nc(-c2ccnc3[nH]ccc23)n1]

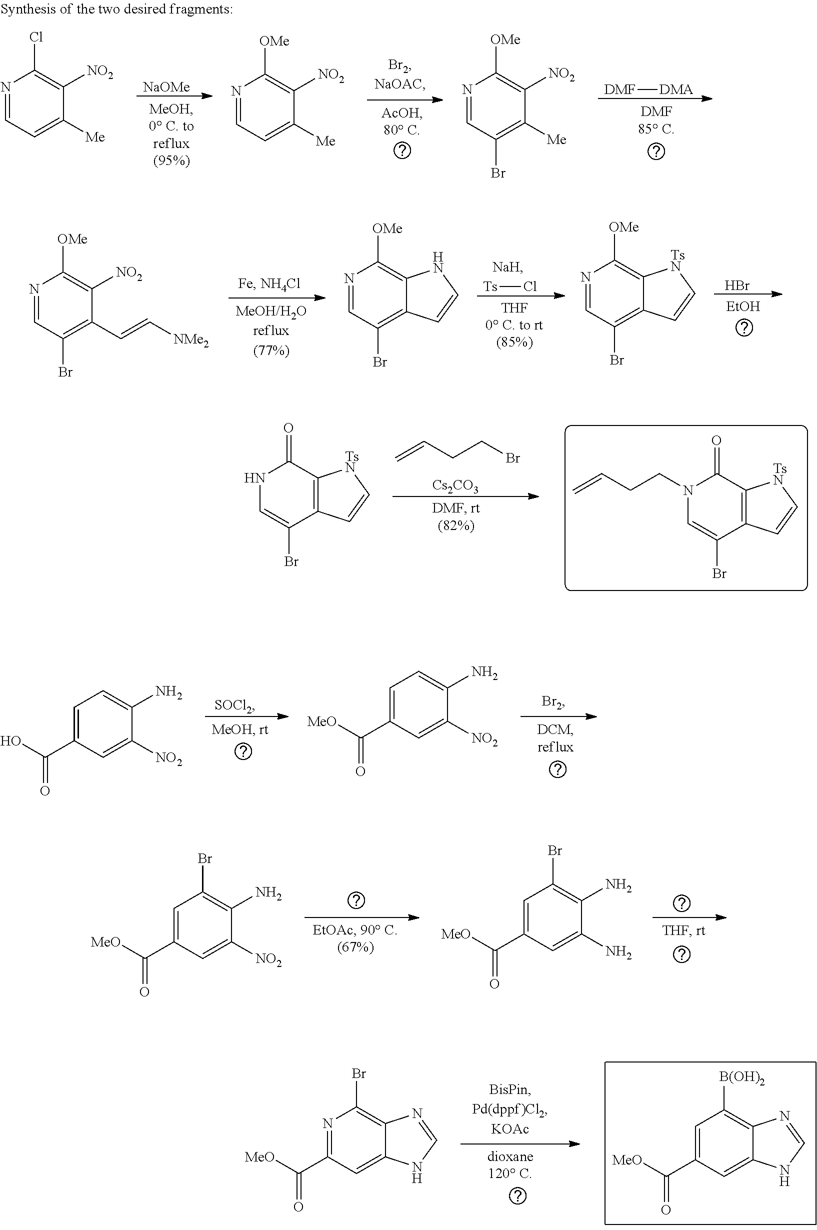

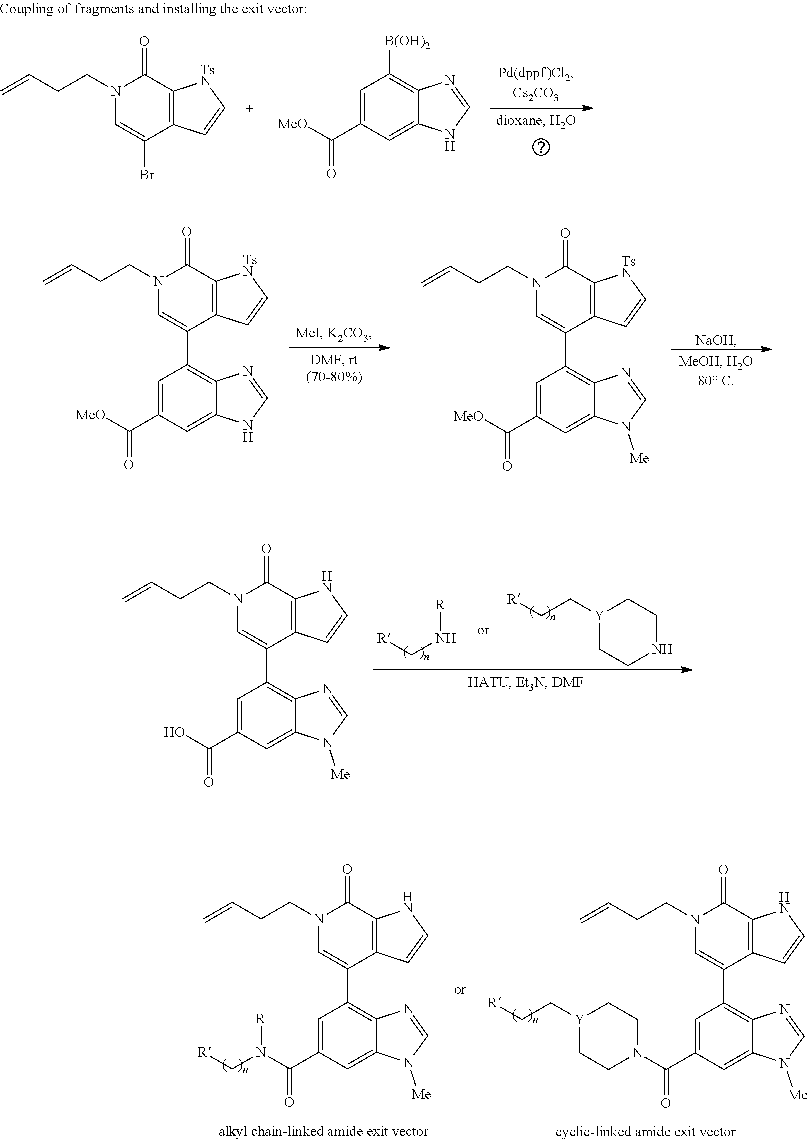

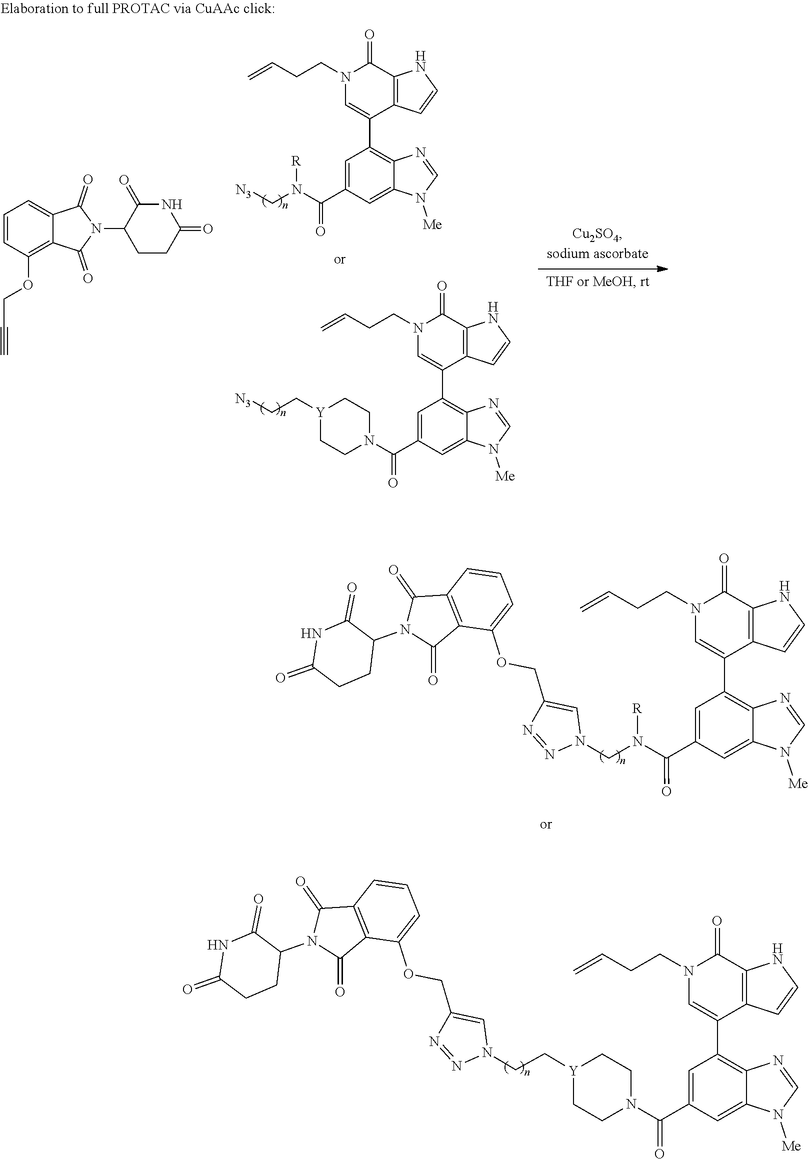

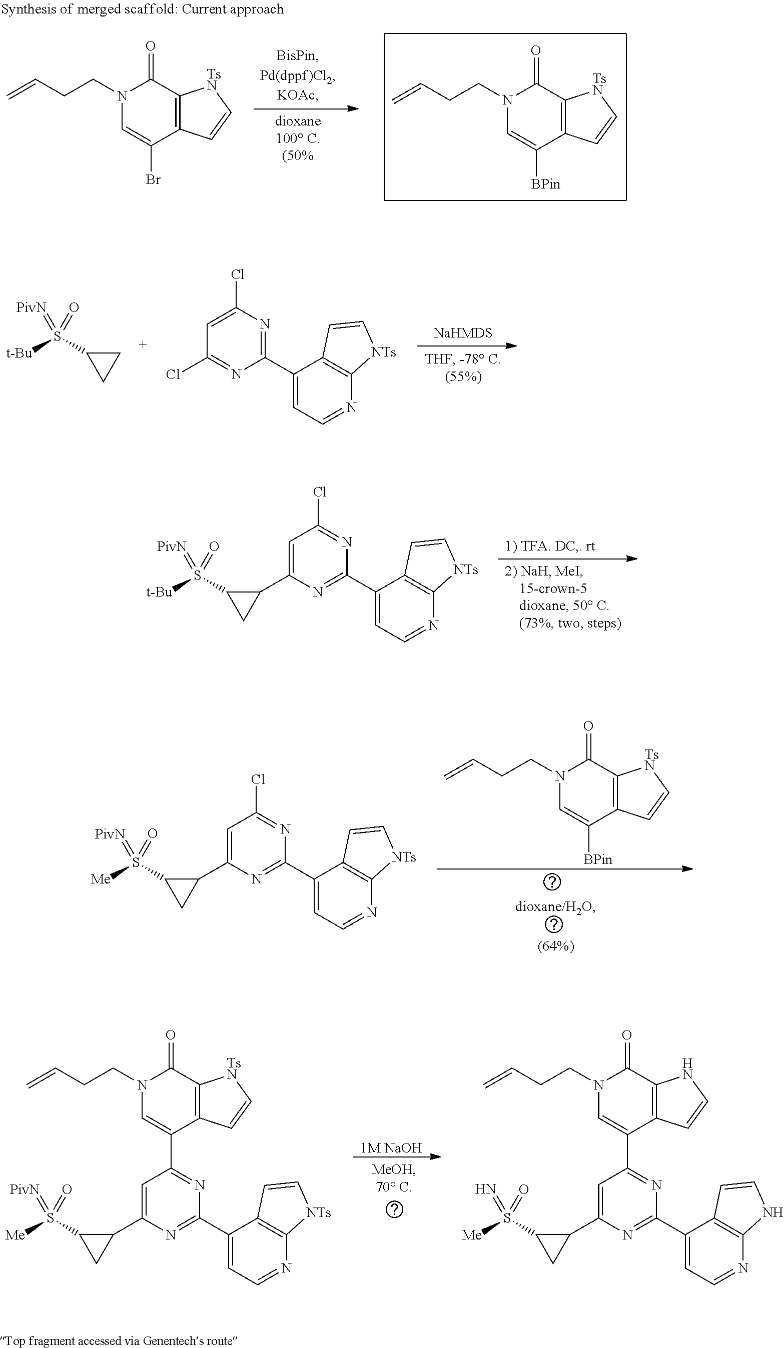

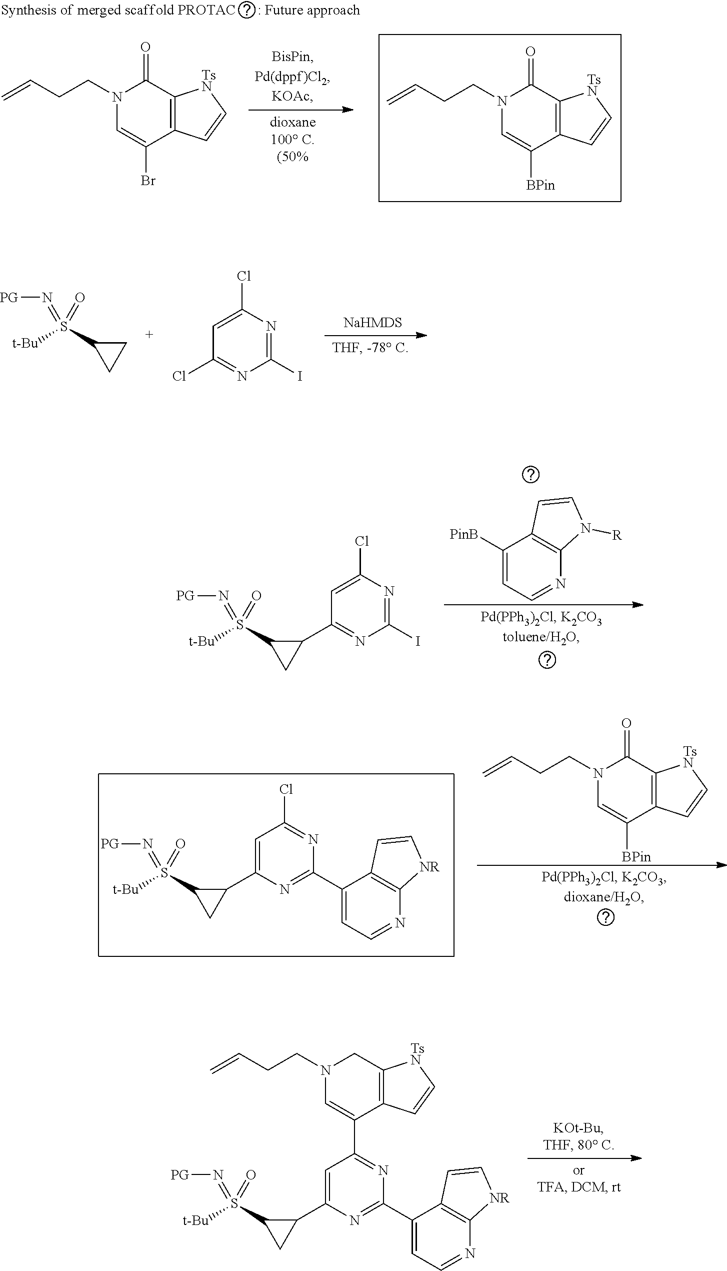

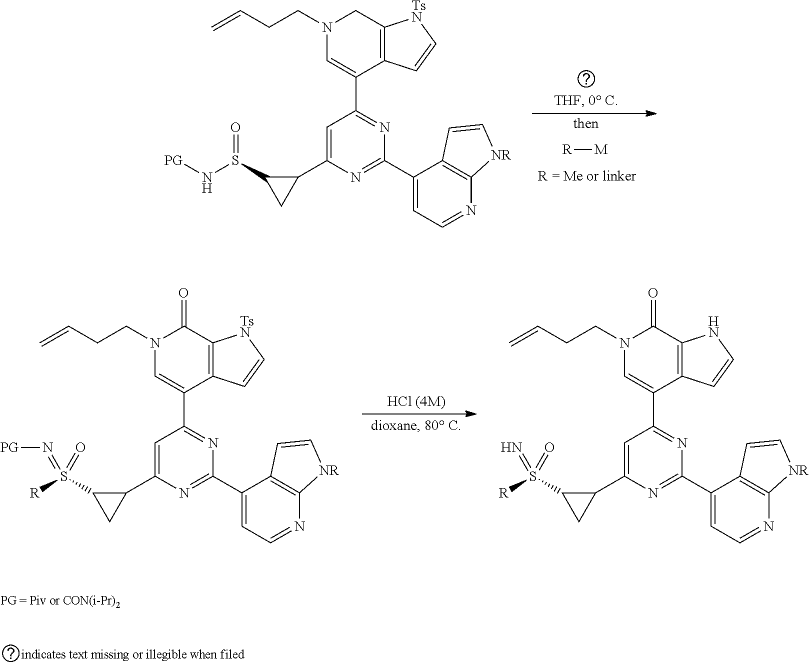

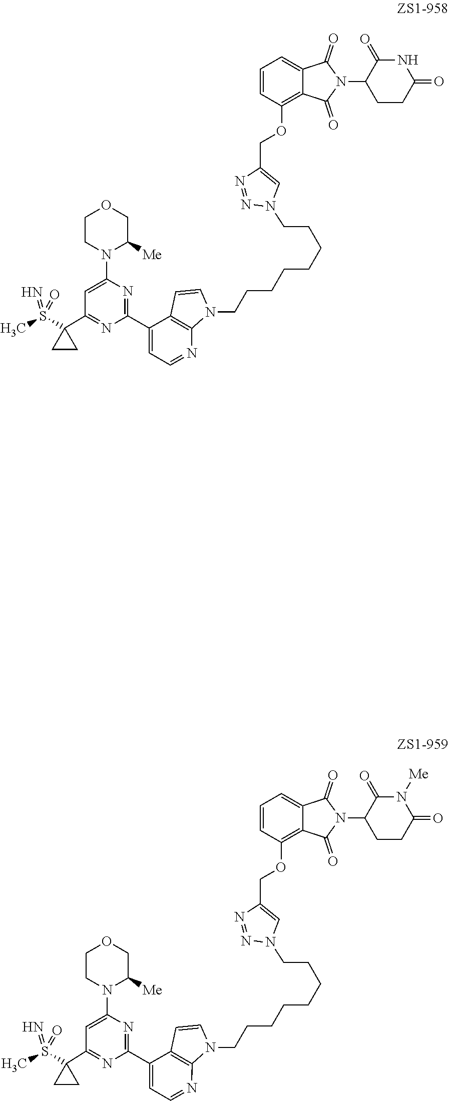 [SMILES: C[C@@H]1COCCN1c1cc(C2([S@](C)(=N)=O)CC2)nc(-c2ccnc3c2ccn3CCCCCCCCn2cc(COc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCCCCCn2cc(COc3cccc4c3C(=O)N(C3CCC(=O)N(C)C3=O)C4=O)nn2)n1]

Synthesis of ceralasertib¹ and PROTACs thereof:

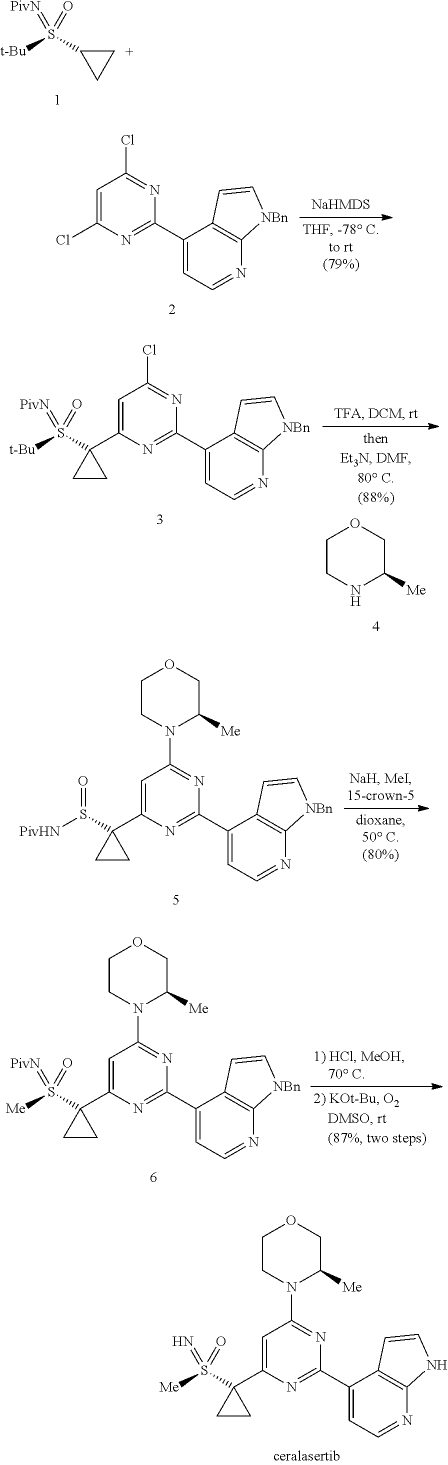

Starting materials for the synthesis of ceralasertib, (S)—N-(tert-butyl(cyclopropyl)(oxo)-λ⁶-sulfaneylidene)pivalamide (1) and 1-benzyl-4-(4,6-dichloropyrimidin-2-yl)-1H-pyrrolo[2,3-b]pyridine (2), were prepared using known literature procedures.¹ The following synthesis was also adopted from our previously published route.¹

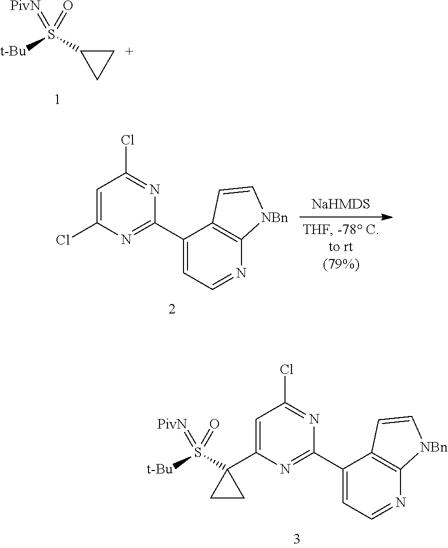

In a septum capped 200 mL round-bottomed flask equipped with a stir bar and argon balloon was added 1 (2.35 g, 9.58 mmol, 1 eq.) and 2 (3.40 g, 9.58 mmol, 1 eq.) and THF (96 mL) then cooled to −78° C. NaHMDS (7.18 mL, 14.37 mmol, 2M in THF, 1.5 eq) was added dropwise and the reaction stirred at −78° C. for 7 hours before gradually warming to room temperature over 1 hour. Once at room temperature the reaction was quenched with saturated aqueous NH₄Cl (100 mL) and the aqueous layer was extracted with EtOAc (4×90 mL), combined organic layers were dried over Na₂SO₄, filtered and adsorbed to silica gel. Purification by silica gel column chromatography using hexanes/EtOAc (0% to 20% EtOAc gradient) provided 3 (4.28 g, 7.59 mmol, 79% yield) as a yellow oil. TLC: R_{f}=0.19 (20% EtOAc in hexanes, UV)¹H NMR: (500 MHz, CDCl₃) δ 8.51 (d, J=5.1 Hz, 1H), 8.17 (d, J=5.0 Hz, 1H), 7.71 (s, 1H), 7.44 (d, J=3.5 Hz, 1H), 7.38 (d, J=3.5 Hz, 1H), 7.34-7.26 (m, 3H), 7.27-7.21 (m, 4H), 5.58 (d, J=3.8 Hz, 2H), 2.58 (ddd, J=10.5, 8.1, 5.9 Hz, 1H), 2.20-2.12 (m, 1H), 1.99 (ddd, J=10.5, 7.9, 5.5 Hz, 1H), 1.47-1.40 (m, 10H), 1.27 (s, 9H) ppm. ¹³C NMR: (126 MHz, CDCl₃) δ 188.03, 165.87, 164.87, 162.03, 149.36, 142.79, 137.45, 134.68, 130.19, 128.77, 127.76, 127.51, 122.69, 118.79, 115.68, 102.12, 66.73, 48.20, 42.32, 42.28, 27.81, 24.66, 16.64, 14.62 ppm. Specific rotation:

$$[ α ] \frac{25}{D} = - 116.6$$

(c 1.00, CHCl₃). HRMS: Calc'd for C₃₀H₃₄ClN₅O₂SNa [M+Na⁺] 586.2014; found: 586.2016.

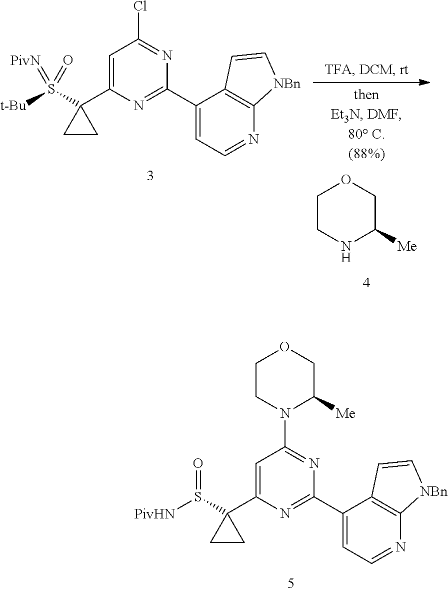

In a septum capped 200 mL round-bottomed flask equipped with a stir bar and argon balloon was added 3 (3.80 g, 6.74 mmol, 1 eq.) and DCM (67 mL). TFA (0.773 mL, 10.1 mmol, 1.5 eq.) was added dropwise then stirred at room temperature for 3.5 hours at which time full conversion was achieved (LC-MS). DCM was removed under reduced pressure then replaced with DMF (50 mL) then 4 (0.818 g, 8.09 mmol, 1.2 eq.) and Et₃N (1.88 mL, 13.5 mmol, 2 eq.) were added. The reaction mixture was heated to 80° C. for 72 hours. The reaction mixture was cooled to room temperature and the solvent removed under reduced pressure to give a crude oil that was taken up in EtOAc (200 mL) and saturated aqueous NH₄Cl (200 mL). The aqueous layer was extracted with EtOAc (3×150 mL), combined organic layers washed with brine (150 mL), dried over Na₂SO₄, filtered and concentrated. Further purification by silica gel column chromatography using hexanes/EtOAc (10% to 60% EtOAc gradient) provided 5 (3.42 g, 5.97 mmol, 88% yield) as a yellow oil. TLC: R_{f}=0.25 (60% EtOAc in hexanes, UV)¹H NMR: (500 MHz, CDCl₃) δ 10.10 (s, 1H), 8.45 (d, J=5.0 Hz, 1H), 7.94 (d, J=5.0 Hz, 1H), 7.33-7.26 (m, 4H), 7.23-7.18 (m, 3H), 6.57 (s, 1H), 5.63 (d, J=15.4 Hz, 1H), 5.51 (d, J=15.4 Hz, 1H), 4.53 (s, 1H), 4.18 (s, 1H), 4.05 (dd, J=11.5, 3.7 Hz, 1H), 3.85 (d, J=11.5 Hz, 1H), 3.75 (dd, J=11.5, 3.1 Hz, 1H), 3.59 (td, J=12.0, 3.0 Hz, 1H), 3.39 (td, J=12.8, 3.9 Hz, 1H), 1.76-1.69 (m, 1H), 1.62-1.56 (m, 1H), 1.45-1.36 (m, 5H), 0.91 (s, 9H) ppm. ¹³C NMR: (126 MHz, CDCl₃) δ 179.41, 164.08, 162.21, 161.40, 149.25, 142.83, 137.78, 137.60, 129.28, 128.71, 127.68, 127.40, 118.77, 115.80, 103.35, 101.83, 70.94, 66.72, 48.06, 47.35, 44.86, 39.39, 26.89, 13.92, 10.43, 10.20 ppm. Specific rotation:

$$[ α ] \frac{23}{D} = - 179.5$$

(c 1.00, CHCl₃). HRMS: Calc'd for C₃₁H₃₇N₆O₃S [M+H⁺] 573.2642; found: 573.2639.

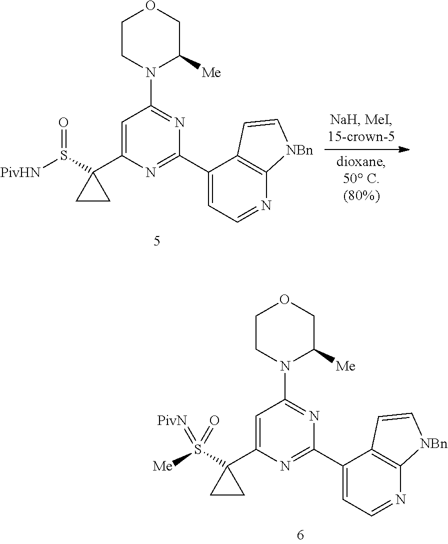

In a septum capped 200 mL round-bottomed flask equipped with a stir bar and argon balloon was added 5 (3.40 g, 5.94 mmol, 1 eq.) and dioxane (60 mL). 15-crown-5 ether (1.41 mL, 7.12 mmol, 1.2 eq.) was added followed by NaH (285 mg, 7.12 mmol, 1.2 eq.) then stirred at room temperature for 20 minutes. Mel (0.742 mL, 11.9 mmol, 2 eq.) was then added and the reaction was heated to 50° C. for 18 hours at which time it was cooled then quenched with saturated aqueous NH₄Cl (75 mL). The aqueous layer was extracted with EtOAc (4×75 mL), combined organic layer washed with brine (2×60 mL), dried over Na₂SO₄, filtered and concentrated. Further purification by silica gel column chromatography using hexanes/EtOAc (0% to 40% EtOAc gradient) provided 6 (2.79 g, 4.75 mmol, 80% yield) as a light-yellow oil. TLC: R_{f}=0.53 (40% EtOAc in hexanes, UV) ¹H NMR: (500 MHz, CDCl₃) δ 8.47 (d, J=5.0 Hz, 1H), 8.04 (d, J=5.0 Hz, 1H), 7.32-7.26 (m, 5H), 7.24-7.21 (m, 2H), 6.78 (s, 1H), 5.57 (s, 2H), 4.62 (s, 1H), 4.12-3.99 (m, 2H), 3.84 (d, J=11.5 Hz, 1H), 3.73 (dd, J=11.5, 3.0 Hz, 1H), 3.57 (td, J=11.9, 3.1 Hz, 1H), 3.45 (s, 3H), 3.44-3.35 (m, 1H), 2.18 (ddd, J=10.4, 7.5, 5.8 Hz, 1H), 1.89 (ddd, J=10.5, 7.3, 5.2 Hz, 1H), 1.70 (ddd, J=9.4, 7.2, 5.8 Hz, 1H), 1.53-1.47 (m, 1H), 1.39 (d, J=6.8 Hz, 3H), 1.07 (s, 9H) ppm. ¹³C NMR: (126 MHz, CDCl₃) δ 188.06, 163.70, 162.12, 161.29, 142.71, 137.66, 129.24, 128.73, 127.68, 118.85, 115.59, 103.46, 101.91, 71.01, 66.71, 48.13, 47.13, 46.54, 41.35, 40.03, 39.54, 27.64, 13.95, 13.10, 12.72 ppm. Specific rotation:

$$[ α ] \frac{23}{D} = - 63.7$$

(c 1.00, CHCl₃). HRMS: Calc'd for C₃₂H₃₉N₆O₃S [M+H⁺]

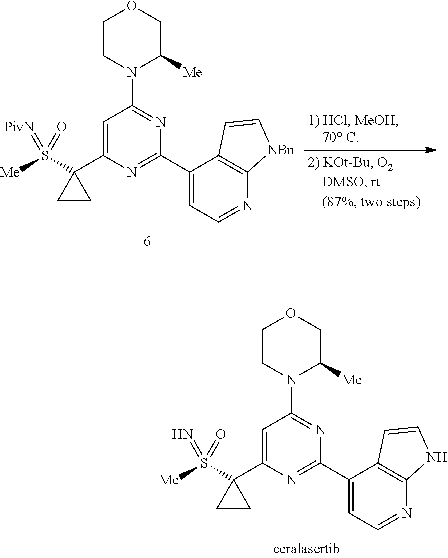

In a septum capped 100 mL round-bottomed flask equipped with a stir bar was added 6 (1.91 g, 3.26 mmol, 1 eq.) and MeOH (33 mL). Once dissolved, HCl (5.43 mL, 32.55 mmol, 6M, 10 eq.) was added and heated to 70° C. for 10 hours then cooled to room temperature. The reaction mixture was made neutral with 1M NaOH and the solvent removed. Aqueous layer extracted with EtOAc (4×75 mL), combined organic layer dried over Na₂SO₄, filtered and concentrated to give a crude oil that was transferred to a septum capped 100 mL round-bottomed flask. DMSO (6.6 mL) and THF (6.6 mL) were added followed by NaH (94 mg, 3.91 mmol, 1.2 eq., 60% wt). The resulting mixture stirred at room temperature for 15 minutes at which time KOt-Bu (40.7 mL, 81.4 mmol, 25 eq., 2M in THF) was added and O₂ (balloon) was bubbled through the reaction mixture for 4 hours then neutralized with 2M HCl. Brine (20 mL) was added, aqueous layer extracted with EtOAc (4×50 mL), combined organic layers washed with brine (25 mL), dried over Na₂SO₄, filtered and concentrated. Further purification by silica gel column chromatography using DCM/MeOH (0% to 5% MeOH gradient) provided ceralasertib (1.17 g, 2.84 mmol, 87% yield) as a golden foam. MTBE (50 mL) was added then stirred for 36 hours and filtered to provide an off-white amorphous solid. TLC: R_{f}=0.24 (5% MeOH in DCM, UV). ¹H NMR: (500 MHz, CDCl₃) δ 10.49 (s, 1H), 8.44 (d, J=5.1 Hz, 1H), 8.07 (d, J=5.1 Hz, 1H), 7.47 (d, J=3.4 Hz, 1H), 7.33 (d, J=3.4 Hz, 1H), 6.91 (s, 1H), 4.53 (s, 1H), 4.17 (d, J=12.3 Hz, 1H), 4.07 (dd, J=11.5, 3.7 Hz, 1H), 3.85 (d, J=11.5 Hz, 1H), 3.76 (dd, J=11.5, 3.1 Hz, 1H), 3.62 (td, J=11.9, 3.1 Hz, 1H), 3.39 (td, J=12.8, 3.9 Hz, 1H), 3.18 (s, 3H), 2.25 (s, 1H), 1.84-1.79 (m, 2H), 1.61-1.54 (m, 2H), 1.39 (d, J=6.8 Hz, 3H) ppm. ¹³C NMR: (126 MHz, CDCl₃) δ 163.66, 162.70, 162.31, 150.27, 142.52, 138.02, 126.20, 118.71, 115.61, 102.84, 102.78, 71.02, 66.77, 48.61, 47.27, 41.32, 39.43, 13.73, 12.73, 12.58 ppm. ¹H NMR: (500 MHz, DMSO) δ 11.79 (s, 1H), 8.33 (d, J=5.0 Hz, 1H), 7.95 (d, J=5.0 Hz, 1H), 7.61-7.54 (m, 1H), 7.22 (dd, J=3.3, 2.0 Hz, 1H), 7.00 (s, 1H), 4.58 (s, 1H), 4.19 (d, J=11.6 Hz, 1H), 4.01 (dd, J=11.4, 3.5 Hz, 1H), 3.80 (d, J=11.4 Hz, 1H), 3.66 (dd, J=11.5, 3.0 Hz, 1H), 3.51 (td, J=11.8, 3.0 Hz, 1H), 3.27 (td, J =13.0, 3.9 Hz, 2H), 3.11 (s, 3H), 1.75 (ddd, J=11.1, 7.7, 3.9 Hz, 1H), 1.58-1.48 (m, 2H), 1.47-1.41 (m, 1H), 1.27 (d, J=6.8 Hz, 3H) ppm. ¹³C NMR: (126 MHz, DMSO) δ 163.70, 163.14, 162.42, 150.64, 142.75, 137.20, 127.80, 118.22, 115.11, 103.37, 102.05, 70.72, 66.50, 48.30, 46.97, 41.59, 39.43, 13.93, 12.74, 11.75 ppm. *Spectroscopic data is in accordance with the literature.² Specific rotation:

$$[ α ] \frac{25}{D} = - 53.7$$

(c 1.00, CHCl₃). HRMS: Calc'd for C₂₀H₂₅N₆O₂S [M+H⁺] 413.1754; found: 413.1748.

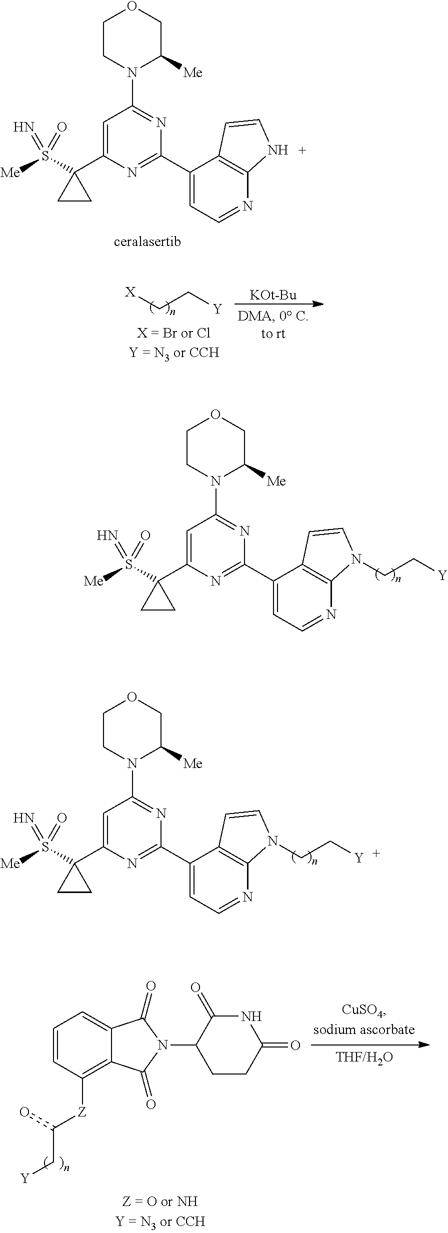

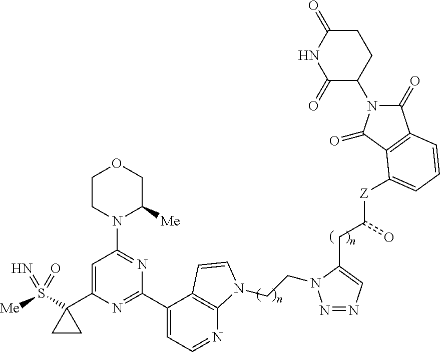

General scheme for the synthesis ceralasertib PROTACs.

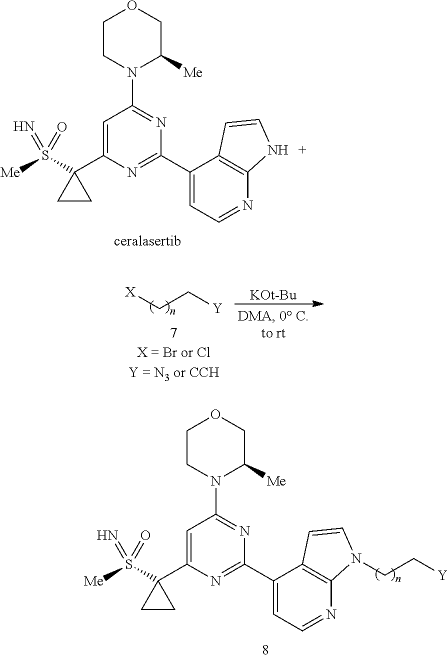

General procedure for the N-alkylation of ceralasertib with alkyl azide or alkyne (7) (GP-1): In a 2-dram septum capped reaction vial equipped with a stir bar and argon balloon was added ceralasertib (1 eq.) and DMA (0.1 M) then cooled to 0° C. KOt-Bu (1.15 eq.) was added, and the temperature maintained at 0° C. for 25 minutes followed by dropwise addition of 7 (1.5 eq.). The reaction mixture stirred at 0° C. for 1 hour then gradually warmed to room temperature where it stirred for an additional 3 hours. The reaction was diluted with EtOAc then acidified to a pH of 3 using 1 M HCl. Additional H₂O was added and extracted with EtOAc, the combined organic layers were dried over Na₂SO₄, filtered and concentrated. Further purification by silica gel column chromatography using DCM/MeOH provides 8.

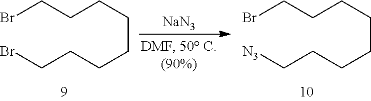 [SMILES: BrCCCCCCCCBr ; [N-]=[N+]=NCCCCCCCCBr ; "NaN3"]

The title compound was prepared according to a known literature procedure.³

In a 20 mL vial equipped with a stir bar was 9 (2.94 g, 10.8 mmol, 2 eq.) in DMF (8.5 mL) at room temperature. NaN₃ (351 mg, 5.40 mmol, 1 eq.) was added and the reaction was heated to 50° C. for 18 hours. The reaction mixture was cool to room temperature, diluted with hexanes (100 mL), washed with water (2×50 mL) and brine (50 mL), dried over Na₂SO₄, filtered and concentrated. Further purification by silica gel column chromatography using hexanes/EtOAc (0% to 2% EtOAc) provided 10 (1.14 g, 4.87 mmol, 90% yield) as a clear colorless oil. TLC: R_{f}=0.26 (100% hexanes, PMA stain). ¹H NMR: (500 MHz, CDCl₃) δ 3.40 (t, J=6.8 Hz, 2H), 3.26 (t, J=6.9 Hz, 2H), 1.90-1.81 (m, 2H), 1.63-1.56 (m, 2H), 1.48-1.40 (m, 2H), 1.40-1.29 (m, 6H) ppm. ¹³C NMR: (126 MHz, CDCl₃) δ 51.45, 33.93, 32.75, 28.97, 28.81, 28.62, 28.05, 26.62 ppm.

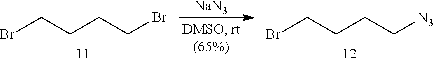 [SMILES: BrCCCCBr ; [N-]=[N+]=NCCCCBr ; "NaN3"]

In a 100 mL round-bottomed flask equipped with a stir bar and septum cap was added 11 (1.87 g, 1.04 mL, 8.68 mmol, 1.15 eq.), DMSO (30 mL) and NaN₃ (491 mg, 7.55 mmol, 1 eq.). The reaction mixture stirred at room temperature for 13 hours then was transferred to a separatory funnel and partitioned between water (150 mL) and hexanes (50 mL). The aqueous layer was extracted with hexanes (3×50 mL), combined organic layer was washed with water (50 mL) and brine (50 mL). Combined organic layer was dried over Na₂SO₄, filtered, and concentrated to give a crude oil that was further purified by silica gel column chromatography using hexanes/EtOAc (0% to 5% EtOAc) to give 12 (876 mg, 4.92 mmol, 65% yield) as a clear colorless oil. ¹H NMR: (500 MHz, CDCl₃) δ 3.44 (t, J=6.5 Hz, 2H), 3.34 (t, J=6.7 Hz, 2H), 2.00-1.92 (m, 2H), 1.83-1.72 (m, 2H) ppm. ¹³C NMR: (126 MHz, CDCl₃) δ 50.61, 32.87, 29.78, 27.49 ppm. Spectroscopic data is in accordance with the literature.⁴

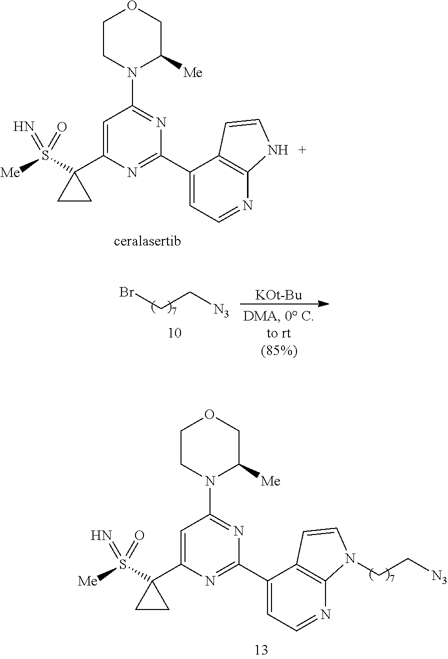

The title compound was prepared using GP-1.

Ceralasertib (75.0 mg, 0.181 mmol, 1 eq.), DMA (1.8 mL, 0.1 M), KOt-Bu (23.5 mg, 0.209 mmol, 1.15 eq.) and 10 (64.0 mg, 0.273 mmol, 1.5 eq.) were used. Purification by silica gel column chromatography using DCM/MeOH (0% to 3% MeOH) provided 13 (87 mg, 0.154 mmol, 85% yield) as a light-yellow oil. TLC: R_{f}=0.36 (5% MeOH in DMC, UV). ¹H NMR: (600 MHz, CDCl₃) δ 8.43 (d, J=5.1 Hz, 1H), 8.02 (d, J=5.0 Hz, 1H), 7.33 (d, J=3.4 Hz, 1H), 7.27 (d, J=3.5 Hz, 1H), 6.90 (s, 1H), 4.54 (s, 1H), 4.34 (t, J=7.2 Hz, 2H), 4.15 (s, 1H), 4.07 (dd, J=11.4, 3.7 Hz, 1H), 3.85 (d, J=11.4 Hz, 1H), 3.77 (dd, J=11.5, 3.0 Hz, 1H), 3.62 (td, J=12.0, 3.0 Hz, 1H), 3.38 (td, J=12.9, 3.9 Hz, 1H), 3.23 (t, J=7.0 Hz, 2H), 3.16 (s, 3H), 1.92-1.86 (m, 2H), 1.82-1.78 (m, 2H), 1.59-1.54 (m, 4H), 1.39 (d, J=6.8 Hz, 3H), 1.36-1.27 (m, 9H) ppm. ¹³C NMR: (126 MHz, CDCl₃) δ 163.79, 162.65, 162.29, 149.05, 142.42, 137.69, 129.15, 118.90, 115.32, 102.73, 101.14, 71.02, 66.77, 51.44, 48.59, 47.25, 44.81, 41.30, 39.42, 30.42, 29.08, 28.99, 28.77, 26.76, 26.60, 13.73, 12.72, 12.56 ppm. HRMS: Calc'd for C₂₈H₄₀N₉O₂S [M+H⁺] 566.3020; found: 566.3030.

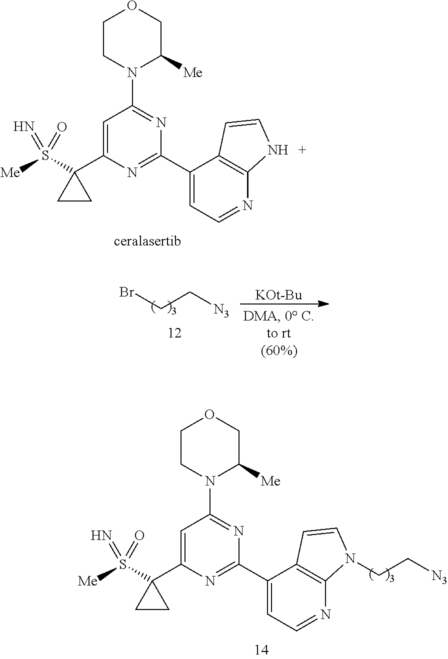

The title compound was prepared using GP-1.

Ceralasertib (155 mg, 0.376 mmol, 1 eq.), 12 (100 mg, 0.564 mmol, 1.5 eq.), and KOt-Bu (49 mg, 0.432 mmol, 1.15. eq.) were used. Purification by silica gel column chromatography using DCM/MeOH (0% to 3% MeOH) provided 14 (115 mg, 0.226 mmol, 60% yield) clear colorless oil. ¹H NMR: (500 MHz, CDCl₃) δ 8.42 (d, J=5.0 Hz, 1H), 8.02 (d, J=5.0 Hz, 1H), 7.32 (d, J=3.4 Hz, 1H), 7.28 (d, J=3.5 Hz, 1H), 6.89 (s, 1H), 4.59-4.48 (m, 1H), 4.38 (t, J=7.0 Hz, 2H), 4.15 (d, J=13.3 Hz, 1H), 4.07 (dd, J=11.5, 3.8 Hz, 1H), 3.85 (d, J=11.5 Hz, 1H), 3.76 (dd, J=11.5, 3.2 Hz, 1H), 3.62 (td, J=11.8, 3.1 Hz, 1H), 3.38 (td, J=12.8, 3.9 Hz, 1H), 3.29 (t, J=6.8 Hz, 2H), 3.16 (s, 3H), 2.03-1.92 (m, 2H), 1.83-1.77 (m, 2H), 1.64-1.54 (m, 4H), 1.38 (d, J=6.8 Hz, 3H) ppm. *Exchangeable N—H not detected. HRMS: Calc'd for C₂₄H₃₂N₉O₂S [M+H⁺] 510.2394; found: 510.2397.

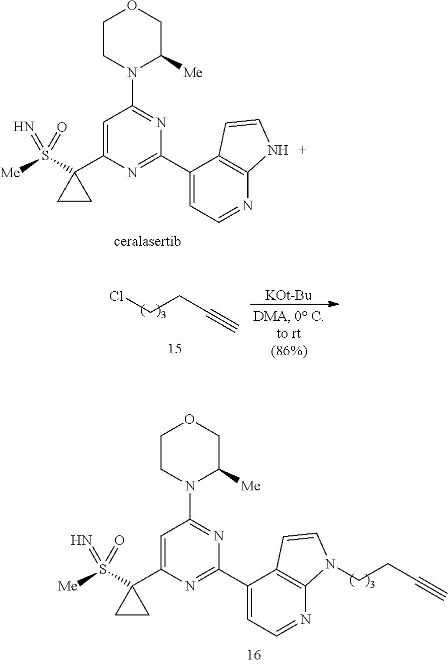

The title compound was prepared using GP-1.

Ceralasertib (79 mg, 0.192 mmol, 1 eq.), 15 (23.4 mg, 0.201 mmol, 1.05 eq.), KOt-Bu (22.6 mg, 0.201 mmol, 1.05. eq.) and KI (47.7 mg, 0.287 mmol, 1.5 eq.) Purification by silica gel column chromatography using DCM/MeOH (0% to 3% MeOH) provided 16 (81 mg, 0.164 mmol, 86% yield) as an amber oil. ¹H NMR: (500 MHz, CDCl₃) δ 8.42 (d, J=5.0 Hz, 1H), 8.02 (d, J=5.0 Hz, 1H), 7.34 (d, J=3.5 Hz, 1H), 7.27 (d, J=3.4 Hz, 1H), 6.90 (s, 1H), 4.58-4.48 (m, 1H), 4.38 (t, J=7.1 Hz, 2H), 4.16 (d, J=12.9 Hz, 1H), 4.07 (dd, J=11.5, 3.8 Hz, 1H), 3.85 (d, J=11.5 Hz, 1H), 3.76 (dd, J=11.5, 3.2 Hz, 1H), 3.62 (td, J=11.9, 3.1 Hz, 1H), 3.38 (td, J=12.8, 3.9 Hz, 1H), 3.16 (s, 3H), 2.44 (s, 1H), 2.23 (td, J=7.0, 2.6 Hz, 2H), 2.09-1.98 (m, 2H), 1.94 (t, J=2.6 Hz, 1H), 1.86-1.75 (m, 2H), 1.60-1.52 (m, 4H), 1.38 (d, J=6.8 Hz, 3H) ppm. ¹³C NMR: (126 MHz, CDCl₃) δ 163.74, 162.65, 162.28, 149.03, 142.45, 137.78, 129.04, 118.91, 115.39, 102.77, 101.37, 83.90, 71.02, 68.75, 66.76, 48.58, 47.24, 44.23, 41.29, 39.42, 29.50, 25.61, 18.09, 13.73, 12.72, 12.56 ppm. HRMS: Calc'd for C₂₆H₃₃N₆O₂S [M+H⁺] 493.2380; found: 493.2384.

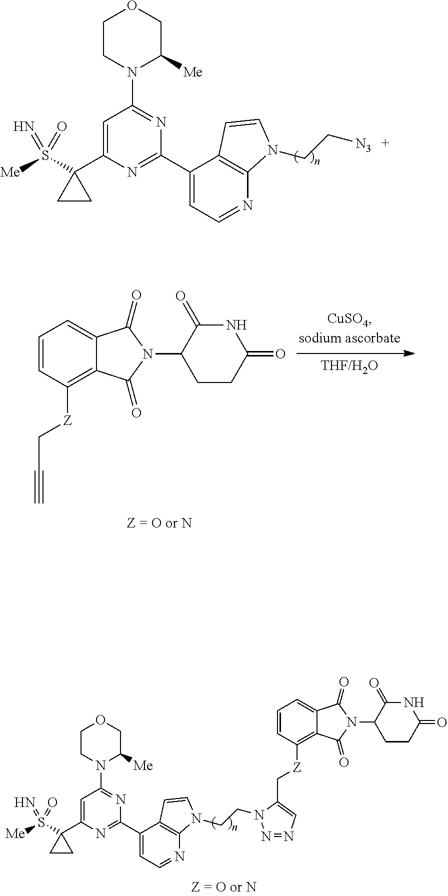

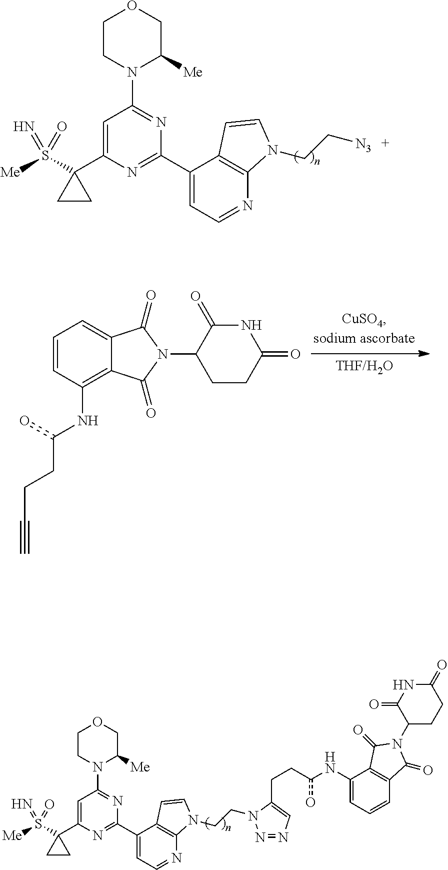

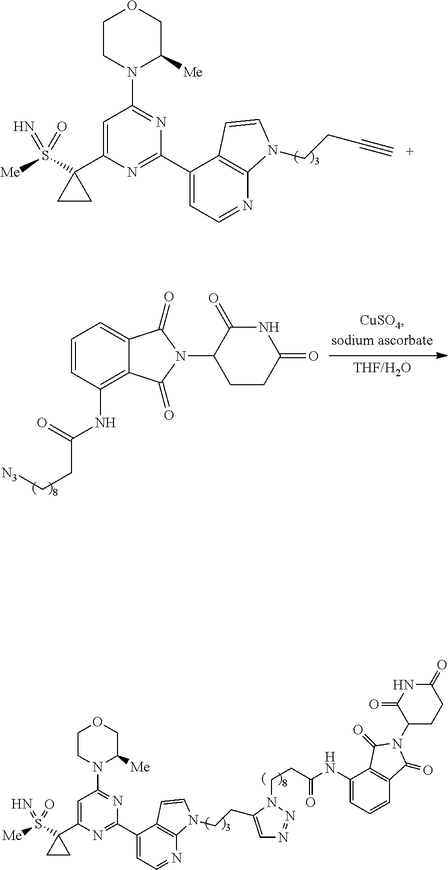

Synthetic schemes for the synthesis of ceralasertib PROTACs.

General procedure for CuAAc PROTAC synthesis (GP-2):

In a 2-dram reaction vial equipped with a stir bar was ceralasertib analog (1 eq.), thalidomide analog (1 eq.), CuSO₄ (0.2 eq.), sodium ascorbate (0.2) eq.), THF and H₂O (˜20:1, 0.1M) under an atmosphere or argon. The reaction stirred at room temperature until full consumption was achieved (determined by TLC and LCMS). Once complete, the reaction was diluted with EtOAc (10 mL), dried over Na₂SO₄, filtered and concentrated. Further purification by preparative TLC or silica gel column chromatography using DCM/MeOH provides PROTACs of greater than 95% purity determined by HPLC.

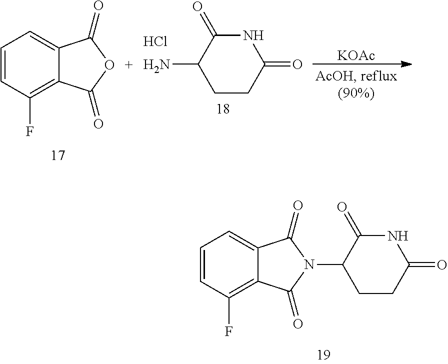 [SMILES: O=C1OC(=O)c2c(F)cccc21 ; Cl ; NC1CCC(=O)NC1=O ; O=C1CCC(N2C(=O)c3cccc(F)c3C2=O)C(=O)N1 ; "KOAc"]

Compound 19 was prepared according to a known literature procedure.⁵

In a 200 mL round-bottomed flask equipped with a stir bar and reflux condenser was 17 (3.05 g, 18.4 mmol, 1 eq.) and 18 (3.32 g, 20.2 mmol, 1.1 eq) in AcOH (30 mL) at room temperature. KOAc (5.41 g, 55.1 mmol, 3 eq.) was added and the reaction mixture was heated to 125° C. for 18 hours. The mixture was cooled to room temperature and AcOH was removed under reduced pressure. H₂O (200 mL) was added, and the resulting suspension stirred at room temperature for 2 hours. The solid was filtered and washed with H₂O (250 mL) and dried to give 19 (4.59 g, 16.6 mmol, 90% yield) as a light-grey solid. ¹H NMR: (500 MHz, DMSO) δ 11.14 (s, 1H), 7.94 (td, J=7.9, 4.5 Hz, 1H), 7.78 (d, J=7.3 Hz, 1H), 7.72 (t, J=8.9 Hz, 1H), 5.15 (dd, J=12.9, 5.4 Hz, 1H), 2.88 (ddd, J=17.5, 14.1, 5.4 Hz, 1H), 2.60 (d, J=17.5 Hz, 1H), 2.56-2.45 (m, 1H), 2.12-2.01 (m, 1H) ppm. ¹⁹F NMR: (471 MHz, DMSO) 6-114.66 ppm. ¹³C NMR: (126 MHz, DMSO) δ 173.21, 170.16, 166.57 (d, J=2.6 Hz), 164.44, 157.28 (d, J=262.3 Hz), 138.53 (d, J=7.9 Hz), 133.92, 123.47 (d, J=19.6 Hz), 120.52 (d, J=3.0 Hz), 117.50 (d, J=12.6 Hz), 49.56, 31.37, 22.31 ppm.

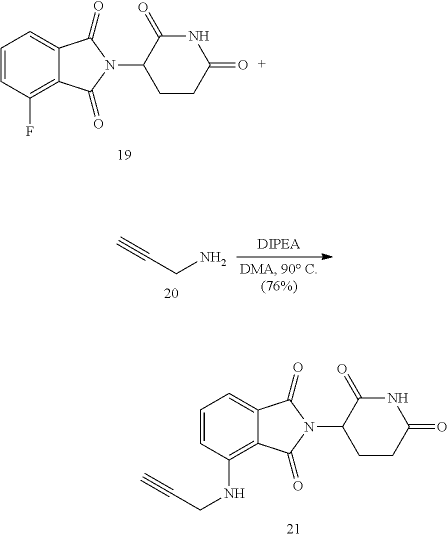

Compound 21 was prepared using a known literature procedure.^{6, 7}

In a 20 mL septum-capped reaction vial equipped with a stir bar was 19 (550 mg, 2.01 mmol, 1 eq.) and 20 (122 mg, 142 μL, 2.21 mmol, 1.1 eq.) in DMA (8 mL) at room temperature. DIPEA (1.05 mL, 6.03 mmol, 3 eq.) was added and the reaction mixture was heated to 90° C. for 19.5 hours. The mixture was cooled to room temperature then quenched with saturated aqueous NH₄Cl (20 mL) and extracted with EtOAc (3×35 mL). The combined organic layers were washed with brine (3×15 mL), dried over Na₂SO₄, filtered and concentrated. Further purification by silica gel column chromatography using hexanes/EtOAc (5% to 50% EtOAc) provided 21 (477 mg, 1.53 mmol, 76% yield) as a yellow oil. ¹H NMR: (500 MHz, CDCl₃) δ 8.17 (s, 1H), 7.57 (dd, J=8.5, 7.2 Hz, 1H), 7.19 (d, J=7.2 Hz, 1H), 7.03 (d, J=8.5 Hz, 1H), 6.45 (t, J=6.2 Hz, 1H), 4.95-4.89 (m, 1H), 4.09 (dd, J=6.1, 2.4 Hz, 2H), 2.80-2.69 (m, 3H), 2.27 (t, J=2.4 Hz, 1H), 2.17 (dq, J=7.4, 2.4 Hz, 1H) ppm.

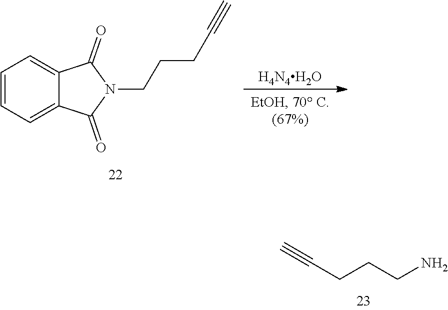

Phthalimide 22 was purchased from Combi-Blocks. The title compound was prepared from a known literature procedure.⁸

In a 200 mL round-bottomed flask equipped with a stir bar and septum capped was 22 (7.40 g, 34.7 mmol, 1 eq.), H₄N₂·H₂O (1.74 g, 34.7 mmol, 1 eq.) in EtOH (70 mL, 0.5 M). The reaction was heated to 70° C. for 2.5 hours then cooled to room temperature. H₂O (50 mL) was added, and the pH of the mixture was adjusted to a pH of 3 using 2 M HCl then filtered and rinsed with H₂O. The filtrate was cooled to 0° C. followed by slow addition of 10 M NaOH (30 mL). The aqueous layer was extracted with DCM (3×100 mL), combined organic layers were dried over Na₂SO₄, filtered and concentrated. Short path distillation (1 atm, 80 to 150° C.) of the crude mixture provided 23 (1.92 g, 23.1 mmol, 66% yield, 105° C.) as a clear colorless oil. ¹H NMR: (500 MHz, CDCl₃) δ 2.85-2.69 (m, 2H), 2.28-2.16 (m, 2H), 1.95-1.89 (m, 1H), 1.69-1.55 (m, 2H), 1.25 (s, 2H) ppm. ¹³C NMR: (126 MHz, CDCl₃) δ 83.95, 68.53, 41.06, 32.09, 15.82 ppm.

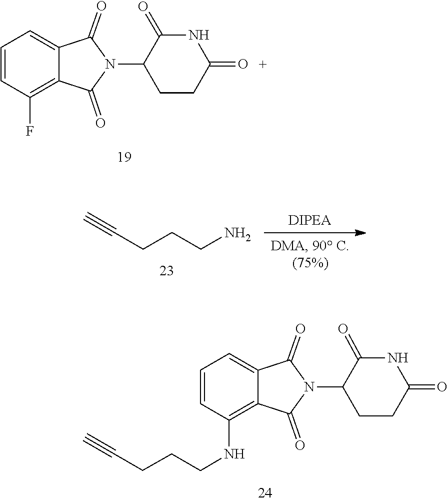

The title compound was prepared according to a similar literature procedure.⁹

In a 20 mL capped vial equipped with a stir bar was 19 (555 mg, 2.01 mmol, 1 eq), 23 (0.184 g, 2.21 mmol, 1.1 eq.) and DMA (8 mL). DIPEA (1.05 mL, 6.03 mmol, 3 eq.) was added and the reaction was heated to 90° C. for 19.5 hours, cooled to room temperature then quenched with saturated aqueous NH₄Cl (15 ml). The aqueous layer was extracted with DCM (4×25 mL), combined organic layers were washed with brine (3×15 mL), dried over Na₂SO₄, filtered and concentrated. Further purification by silica gel column chromatography using hexanes/EtOAc (0% to 60%) provided 24 (508 mg, 1.50 mmol, 75% yield) as a yellow oil. ¹H NMR: (500 MHz, CDCl₃) δ 8.40 (s, 1H), 7.49 (dd, J=8.5, 7.1 Hz, 1H), 7.09 (d, J=7.0 Hz, 1H), 6.93 (d, J=8.6 Hz, 1H), 6.30 (s, 1H), 4.91 (dd, J=12.3, 5.3 Hz, 1H), 3.43 (t, J=7.0 Hz, 2H), 2.90-2.84 (m, 1H), 2.81-2.68 (m, 2H), 2.33 (td, J=6.8, 2.7 Hz, 2H), 2.15-2.08 (m, 1H), 2.02 (t, J=2.6 Hz, 1H), 1.88-1.84 (m, 2H) ppm. ¹³C NMR: (126 MHz, CDCl₃) δ 171.23, 169.50, 168.48, 167.60, 146.86, 136.18, 132.53, 116.62, 111.65, 110.13, 83.01, 69.55, 48.89, 41.35, 31.42, 27.84, 22.80, 15.97 ppm.

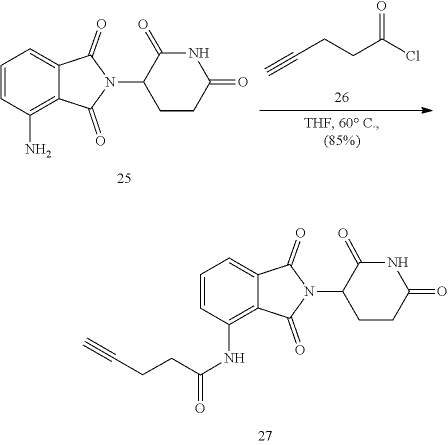

Pomalidomide (25) was purchased from Combi-Blocks. Acid chloride 26 was prepared according to known literature procedure from the corresponding commercial carboxylic acid.¹⁰ The title compound was prepared according to a known literature procedure.¹

In a 20 mL septum capped vial equipped with a stir bar was 25 (0.10 g, 0.37 mmol, 1 eq.) and 26 (85.3 m, 0.732 mmol, 2 eq.) in THF (4 mL). The mixture was heated to 60° C. for 12 hours then cooled to room temperature and solvent removed under reduced pressure. The crude material was taken up in EtOAc and filtered through a short silica plug while rinsing with EtOAc to provide 27 (110 mg, 0.311 mmol, 85% yield) as a yellow solid that was used without further purification. ¹H NMR: (500 MHz, CDCl₃) δ 9.46 (s, 1H), 8.84 (dd, J=8.5, 0.8 Hz, 1H), 8.28 (s, 1H), 7.76-7.69 (m, 1H), 7.56 (dd, J=7.3, 0.8 Hz, 1H), 4.96 (dd, J=12.3, 5.4 Hz, 1H), 2.98-2.88 (m, 1H), 2.84-2.75 (m, 2H), 2.73-2.68 (m, 2H), 2.66-2.63 (m, 2H), 2.21-2.15 (m, 1H), 2.03 (t, J=2.6 Hz, 1H) ppm. ¹³C NMR: (126 MHz, CDCl₃) δ 175.83, 170.75, 170.17, 167.86, 166.63, 137.57, 136.52, 131.13, 125.41, 118.71, 115.46, 82.09, 69.78, 49.30, 36.65, 32.85, 22.68, 14.48 ppm.

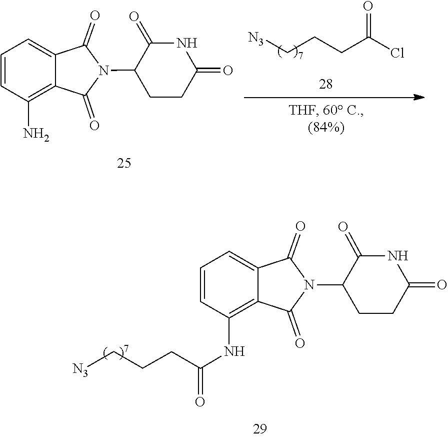

Acid chloride 28 was prepared in two steps using a known literature procedure from the corresponding commercial carboxylic acid.¹²

In a 2-dram capped vial equipped with a stir bar was 25 (0.096 g, 0.35 mmol, 1 eq.) and 28 (326 mg, 1.41 mmol, 4 eq.) in THF (3.5 mL). The reaction was heated to 60° C. for 3 hours, cooled to room temperature then quenched with saturated aqueous NaHCO₃ (8 mL). The aqueous layer was extracted with EtOAc (4×15 mL), combined organic layers were dried over Na₂SO₄, filtered and concentrated. Further purification by silica gel column chromatography using DCM/MeOH (0% to 2% MeOH) provided 29 (139 mg, 0.297 mmol, 84% yield) as a light-yellow solid. ¹H NMR: (500 MHz, CDCl₃) δ 9.40 (s, 1H), 8.82 (d, J=8.5 Hz, 1H), 8.68 (s, 1H), 7.69 (t, J=7.9 Hz, 1H), 7.52 (d, J=7.3 Hz, 1H), 5.00-4.88 (m, 1H), 3.24 (t, J=7.0 Hz, 2H), 2.93-2.86 (m, 1H), 2.82-2.70 (m, 2H), 2.44 (t, J=7.6 Hz, 2H), 2.19-2.11 (m, 1H), 1.73 (p, J=7.4 Hz, 2H), 1.59-1.55 (m, 2H), 1.39-1.29 (m, 10H) ppm. ¹³C NMR: (126 MHz, CDCl₃) δ 172.47, 171.17, 169.19, 168.16, 166.73, 137.90, 136.46, 131.09, 125.32, 118.42, 115.26, 51.46, 49.26, 37.96, 31.37, 29.23, 29.15, 29.06, 29.05, 28.80, 26.67, 25.20, 22.68 ppm.

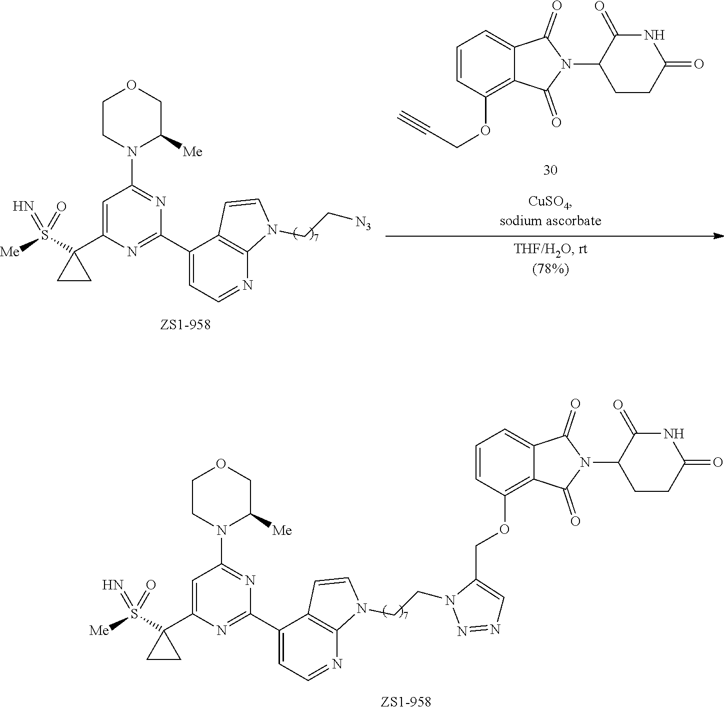

The title compound was prepared using GP-2. Propargyl thalidomide analog (30) was purchased by Matrix Scientific.

13 (17.0 mg, 30.1 μmol, 1 eq.), 30 (9.40 mg, 30.1 μmol, 1 eq.), CuSO₄ (1.0 mg, 6.0 μmol, 0.2 eq.), sodium ascorbate (1.2 mg, 6.0 μmol, 0.2 eq.), THF (0.4 mL) and H₂O (2 drops). Purification by preparative TLC using DCM/MeOH (5% MeOH) provided ZS1-958 (20.6 mg, 23.5 μmol, 78% yield) as a light-yellow solid. TLC: R_{f}=0.36 (5% MeOH in DMC, UV). ¹H NMR: (500 MHz, CDCl₃) δ 8.54 (s, 1H), 8.42 (d, J=5.0 Hz, 1H), 8.01 (d, J=5.0 Hz, 1H), 7.71 (s, 1H), 7.67 (dd, J=8.5, 7.3 Hz, 1H), 7.50-7.45 (m, 2H), 7.31 (d, J=3.4 Hz, 1H), 7.26-7.25 (m, 1H), 6.88 (s, 1H), 5.46 (s, 2H), 4.94 (dd, J=12.1, 5.4 Hz, 1H), 4.59-4.48 (m, 1H), 4.32 (q, J=6.7 Hz, 4H), 4.16 (d, J=13.0 Hz, 1H), 4.07 (dd, J=11.5, 3.7 Hz, 1H), 3.85 (d, J=11.5 Hz, 1H), 3.76 (dd, J=11.5, 3.0 Hz, 1H), 3.65-3.59 (m, 1H), 3.41-3.36 (m, 1H), 3.18 (s, 3H), 2.93-2.65 (m, 4H), 1.88-1.84 (m, 4H), 1.80 (dd, J=5.1, 3.1 Hz, 2H), 1.57-1.54 (m, 2H), 1.38 (d, J=6.8 Hz, 3H), 1.27 (d, J=15.2 Hz, 9H) ppm. HRMS: Calc'd for C₄₄H₅₂N₁₁O₇S [M+H⁺] 878.3766; found: 878.3761.

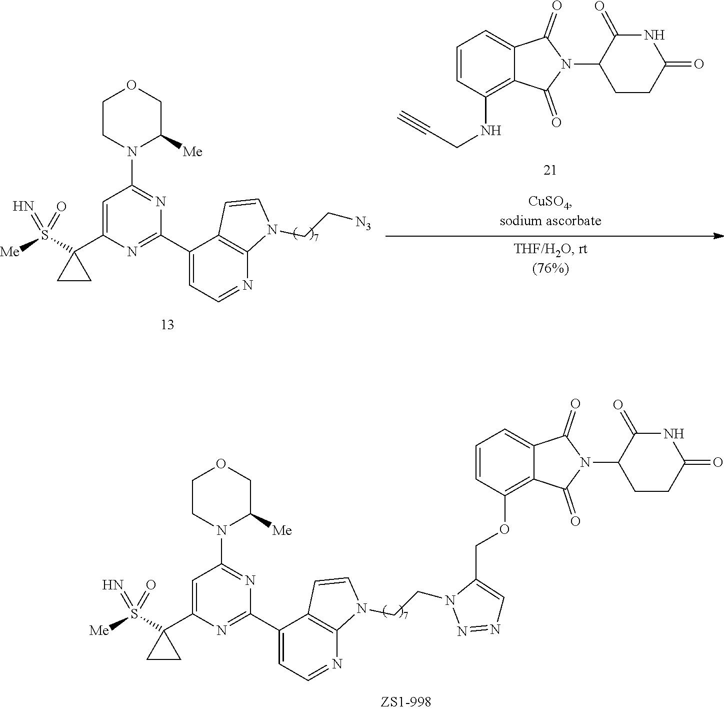

The title compound was prepared using GP-2.

13 (35.0 mg, 61.9 μmol, 1 eq.), 21 (19.3 mg, 61.9 μmol, 1 eq.), sodium ascorbate (2.5 mg, 12 μmol, 0.2 eq.), CuSO₄ (2 mg, 12 mol, 0.2 eq.) were used. Purification by preparative TLC using DCM/MeOH (5% MeOH) provided ZS1-998 (41.1 mg, 46.9 μmol, 76% yield) as a yellow solid. ¹H NMR: (500 MHz, CDCl₃) δ 8.52 (s, 1H), 8.42 (d, J=5.2 Hz, 1H), 8.05 (d, J=5.1 Hz, 1H), 7.49-7.43 (m, 2H), 7.35-7.28 (m, 2H), 7.12 (d, J=7.1 Hz, 1H), 6.98 (d, J=8.5 Hz, 1H), 6.91 (s, 1H), 6.67 (t, J=5.9 Hz, 1H), 4.90 (ddd, J=12.2, 5.1, 1.5 Hz, 1H), 4.63 (d, J=6.0 Hz, 2H), 4.58-4.47 (m, 1H), 4.37 (t, J=7.0 Hz, 2H), 4.29 (t, J=7.3 Hz, 2H), 4.21-4.12 (m, 1H), 4.08 (dd, J=11.5, 3.5 Hz, 1H), 3.86 (d, J=11.5 Hz, 1H), 3.76 (dd, J=11.5, 3.0 Hz, 1H), 3.65-3.58 (m, 1H), 3.38 (td, J=12.8, 3.9 Hz, 1H), 3.19 (s, 3H), 2.89-2.69 (m, 3H), 2.11 (ddt, J=11.7, 9.6, 5.8 Hz, 1H), 1.88-1.80 (m, 5H), 1.38 (d, J=6.8 Hz, 3H), 1.32-1.24 (m, 12H) ppm. HRMS: Calc'd for C₄₄H₅₃N₁₂O₆S [M+H⁺] 877.3926; found: 877.3928.

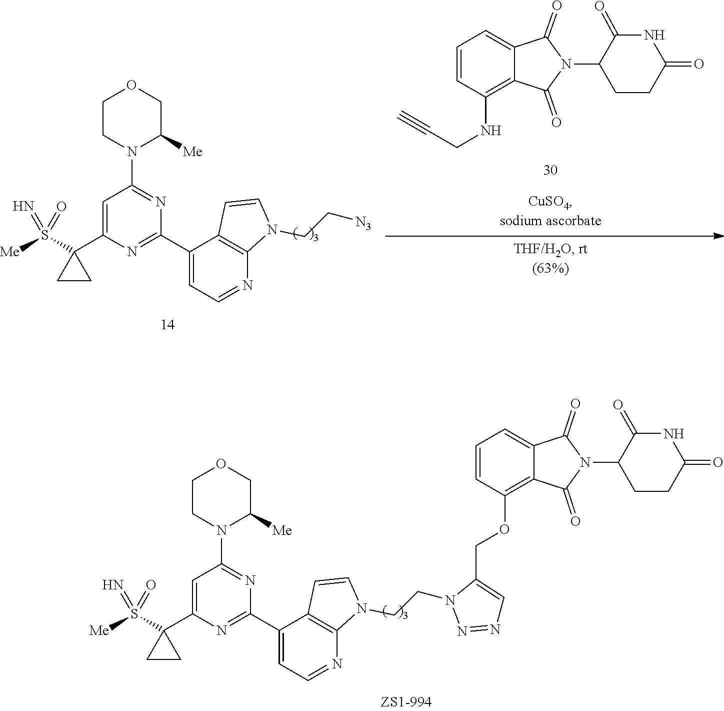

14 (78 mg, 153 μmol, 1 eq.), 30 (47.8 mg, 153 μmol, 1 eq.), sodium ascorbate (6.1 mg, 31 μmol, 0.2 eq.), CuSO₄ (4.9 mg, 31 μmol, 0.2 eq.) were used. Purification by silica gel column chromatography using DCM/MeOH (5% MeOH) provided ZS1-994 (79 mg, 96.1 μmol, 63% yield) as a beige solid. ¹H NMR: (500 MHz, CDCl₃) δ 8.77 (d, J=6.4 Hz, 1H), 8.42 (dd, J=5.0, 1.1 Hz, 1H), 8.06 (dd, J=5.2, 3.4 Hz, 1H), 7.73 (d, J=19.0 Hz, 1H), 7.66 (dd, J=8.5, 7.2 Hz, 1H), 7.49-7.44 (m, 2H), 7.31-7.27 (m, 2H), 6.89 (d, J=2.0 Hz, 1H), 5.44 (d, J=2.6 Hz, 2H), 4.91 (td, J=12.1, 5.4 Hz, 1H), 4.59-4.49 (m, 1H), 4.45-4.37 (m, 4H), 4.08 (dd, J=11.5, 3.7 Hz, 1H), 3.86 (d, J=11.5 Hz, 1H), 3.76 (dd, J=11.5, 3.2 Hz, 1H), 3.67-3.58 (m, 2H), 3.38 (tt, J=12.7, 3.5 Hz, 1H), 3.19 (d, J=3.1 Hz, 3H), 2.90-2.68 (m, 4H), 2.13-2.08 (m, 2H), 1.99-1.89 (m, 5H), 1.85-1.78 (m, 2H), 1.38 (d, J=6.8 Hz, 3H) ppm. HRMS: Calc'd for C₄₀H₄₄N₁₁O₇S [M+H⁺] 822.3140; found: 822.3148.

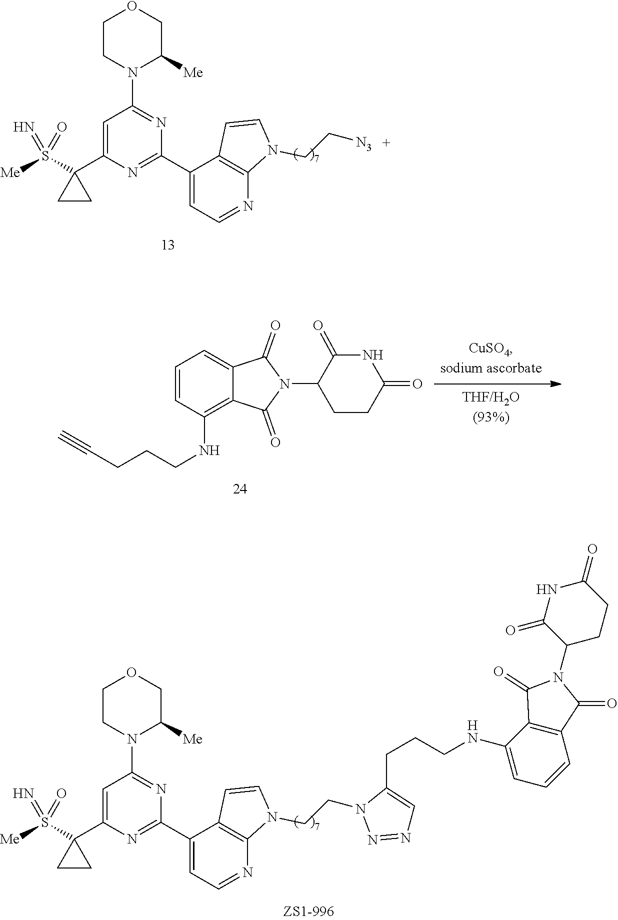

The title compound was prepared using GP-2:

13 (35.0 mg, 61.9 μmol, 1 eq.), 24 (21.0 mg, 62.0 μmol, 1 eq.), sodium ascorbate (2.5 mg, 12 μmol, 0.2 eq.), CuSO₄ (2 mg, 12 μmol, 0.2 eq.) were used. Purification by preparative TLC using DCM/MeOH (5% MeOH) provided ZS1-996 (52.0 mg, 61.9 μmol, 93% yield) as a yellow solid with >95% purity determined by HPLC. ¹H NMR: (500 MHz, CDCl₃) δ 8.42 (d, J=5.2 Hz, 1H), 8.31 (s, 1H), 8.07 (d, J=5.1 Hz, 1H), 7.47 (t, J=7.8 Hz, 1H), 7.36-7.28 (m, 3H), 7.08 (d, J=7.1 Hz, 1H), 6.93 (s, 1H), 6.88 (d, J=8.5 Hz, 1H), 6.29 (s, 1H), 4.89 (dt, J=12.1, 4.3 Hz, 1H), 4.59-4.47 (m, 1H), 4.40 (t, J=6.9 Hz, 2H), 4.28 (t, J=7.2 Hz, 2H), 4.20-4.13 (m, 1H), 4.08 (dd, J=11.5, 3.4 Hz, 1H), 3.86 (d, J=11.5 Hz, 1H), 3.76 (dd, J=11.5, 2.8 Hz, 1H), 3.62 (td, J=11.9, 2.6 Hz, 1H), 3.38 (ddd, J=23.0, 12.6, 5.1 Hz, 3H), 3.18 (s, 3H), 2.85-2.68 (m, 5H), 2.15-2.09 (m, 1H), 2.08-2.03 (m, 2H), 1.86 (dt, J=21.4, 6.7 Hz, 6H), 1.38 (d, J=6.8 Hz, 3H), 1.33-1.25 (m, 12H) ppm. HRMS: Calc'd for C₄₆H₅₇N₁₂O₆S [M+H⁺] 905.4239; found: 905.4245.

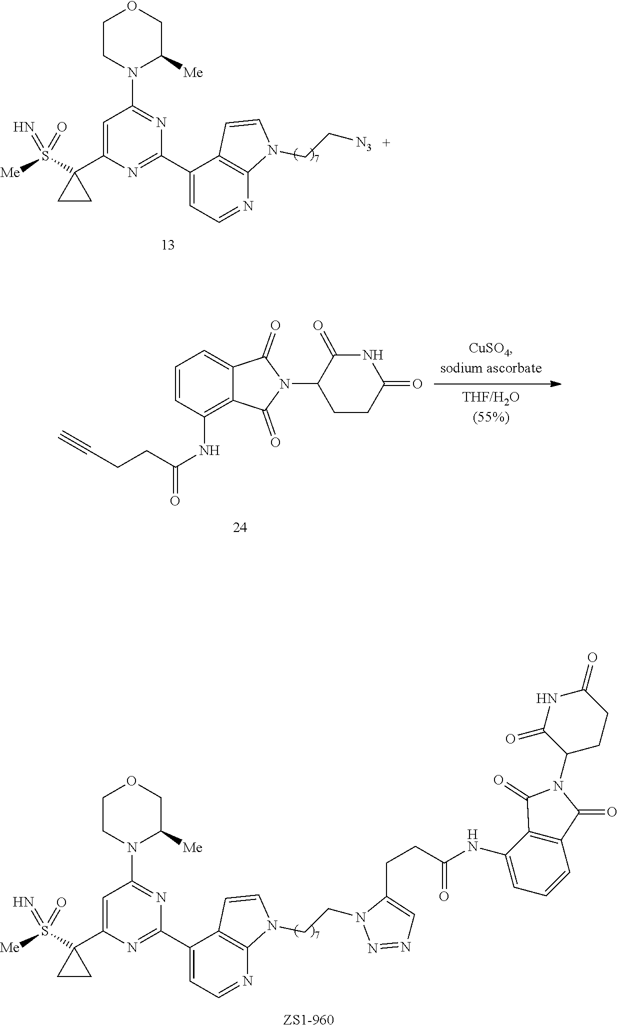

The title compound was prepared using GP-2:

13 (20.0 mg, 35.4 μmol, 1 eq.), 27 (12.5 mg, 35.4 μmol, 1 eq.), sodium ascorbate (1.4 mg, 7.1 μmol, 0.2 eq.), CuSO₄ (1.4 mg, 7.1 μmol, 0.2 eq.) were used. Purification by preparative TLC using DCM/MeOH (5% MeOH) provided ZS1-960 (18.0 mg, 19.6 μmol, 55% yield) as a light-yellow solid. ¹H NMR: (500 MHz, CDCl₃) δ 9.42 (s, 1H), 8.80 (dd, J=8.5, 0.8 Hz, 1H), 8.42 (d, J=5.0 Hz, 2H), 8.04-7.99 (m, 1H), 7.69 (dd, J=8.5, 7.3 Hz, 1H), 7.53 (dd, J=7.3, 0.8 Hz, 1H), 7.34 (s, 1H), 7.31 (d, J=3.5 Hz, 1H), 6.88 (s, 1H), 4.96-4.89 (m, 1H), 4.59-4.48 (m, 1H), 4.32 (t, J=7.1 Hz, 2H), 4.26 (t, J=7.2 Hz, 2H), 4.16 (d, J=13.0 Hz, 1H), 4.07 (dd, J=11.5, 3.8 Hz, 1H), 3.86 (d, J=11.5 Hz, 1H), 3.76 (dd, J=11.5, 3.2 Hz, 1H), 3.66-3.60 (m, 3H), 3.45-3.34 (m, 1H), 3.18 (s, 3H), 3.15 (t, J=7.2 Hz, 2H), 2.93-2.90 (m, 2H), 2.82-2.70 (m, 2H), 2.20-2.11 (m, 1H), 1.88-1.79 (m, 5H), 1.58-1.54 (m, 2H), 1.38 (d, J=6.8 Hz, 3H), 1.30-1.26 (m, 1OH) ppm. HRMS: Calc'd for C₄₆H₅₅N₁₂O₇S [M+H⁺] 919.4032; found: 919.4015.

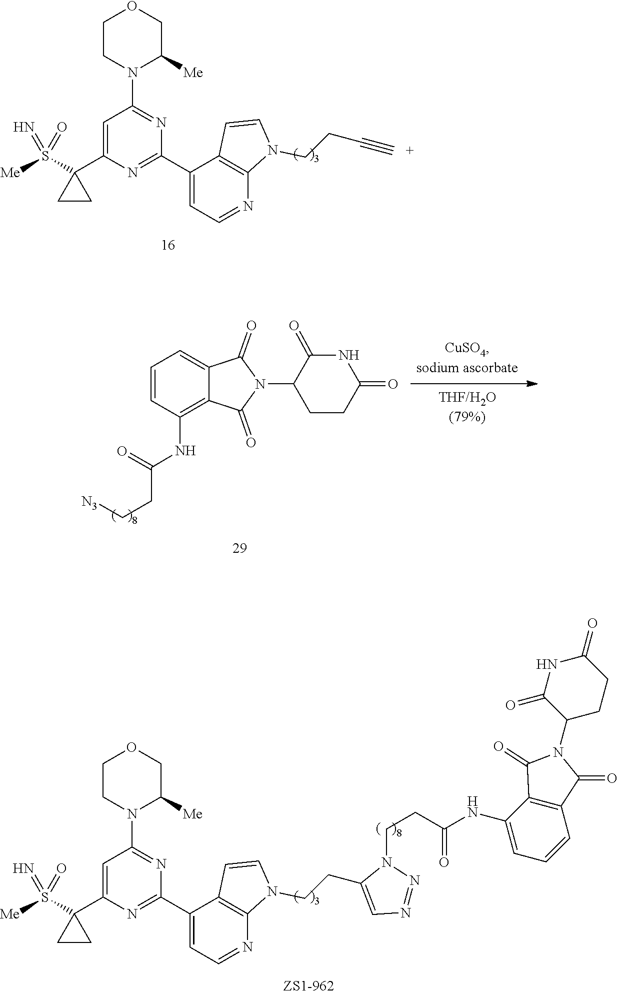

The title compound was prepared using GP-2.

16 (22.0 mg, 44.7 μmol, 1 eq.), 29 (20.9 mg, 44.7 μmol, 1 eq.), sodium ascorbate (1.8 mg, 8.9 μmol, 0.2 eq.), CuSO₄ (1.4 mg, 8.9 μmol, 0.2 eq.) were used. Purification by silica gel column chromatography using DCM/MeOH (0% to 5% MeOH) provided ZS1-962 (34 mg, 35.4 μmol, 79% yield) as a yellow oil that was not >95% pure by HPLC. Further purification by preparative TLC using 5% MeOH in DCM provided ZS1-962 with >95% purity determined by HPLC. ¹H NMR: (500 MHz, CDCl₃) δ 9.41 (s, 1H), 8.86-8.79 (m, 1H), 8.52 (s, 1H), 8.42 (d, J=5.1 Hz, 1H), 8.03 (d, J=5.1 Hz, 1H), 7.70 (dd, J=8.6, 7.3 Hz, 1H), 7.54 (dd, J=7.3, 0.7 Hz, 1H), 7.33 (d, J=3.5 Hz, 1H), 7.27 (d, J=3.5 Hz, 1H), 7.22 (s, 1H), 6.91 (s, 1H), 4.99-4.91 (m, 1H), 4.60-4.49 (m, 1H), 4.40 (t, J=7.1 Hz, 2H), 4.26 (t, J=7.2 Hz, 2H), 4.14 (dd, J=12.7, 7.9 Hz, 1H), 4.07 (dd, J=11.5, 3.8 Hz, 1H), 3.85 (d, J=11.5 Hz, 1H), 3.76 (dd, J=11.5, 3.2 Hz, 1H), 3.62 (td, J=11.9, 3.0 Hz, 1H), 3.38 (td, J=12.9, 4.0 Hz, 1H), 3.17 (s, 3H), 2.94-2.72 (m, 5H), 2.44 (t, J=7.5 Hz, 2H), 2.20-2.13 (m, 1H), 1.97 (p, J=7.3 Hz, 2H), 1.87-1.79 (m, 4H), 1.73 (p, J=8.2, 7.6 Hz, 4H), 1.56 (dd, J=4.7, 2.1 Hz, 1H), 1.38 (d, J=6.8 Hz, 3H), 1.35-1.21 (m, 12H) ppm. HRMS: Calc'd for C₄₉H₆₁N₁₂O₇S [M+H⁺] 961.4501; found: 961.4491.

### Synthesis of GNE-371^{13,14} and PROTACs Thereof:

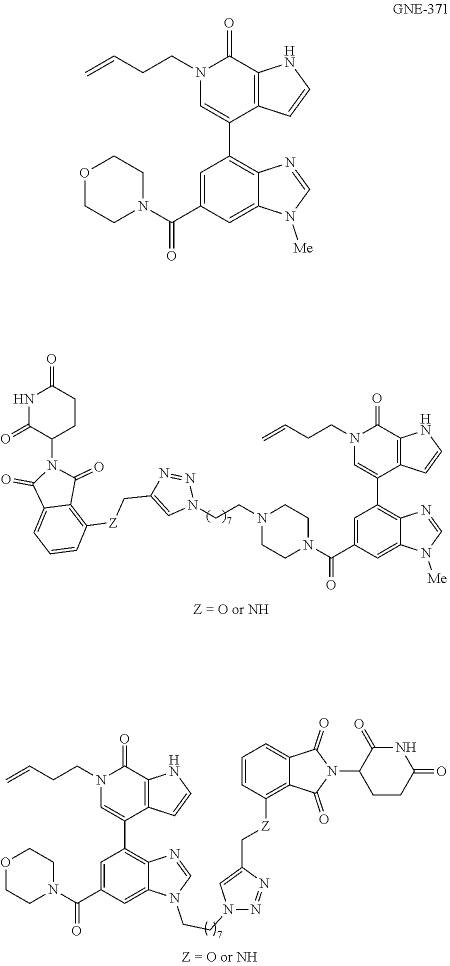

The synthesis of GNE-371 was performed using previously reported methods.^{13, 14}

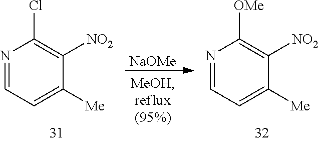 [SMILES: Cc1ccnc(Cl)c1[N+](=O)[O-] ; COc1nccc(C)c1[N+](=O)[O-] ; "NaOMe"]

In a septum capped 500 mL round-bottomed flask equipped with a stir bar, addition funnel and argon balloon were added NaOMe (25.0 g, 464 mmol, 3.2 eq.) and MeOH (85 mL) then cooled 0° C. 2-chloro-4-methyl-3-nitropyridine (31) (25.0 g, 145 mmol, 1 eq.) was dissolved in MeOH (150 mL) and transferred to the addition funnel then added dropwise over 2 hours. Once addition was complete, the reaction mixture was heated to 78° C. (reflux) for 24 hours. Cooled to room temperature, solvent removed under reduced until approximately 90 mL remained pressure then water was added (150 mL) and stirred for 5 minutes. The resulting precipitate was filtered through a sintered glass funnel while rinsing with water. An off-white solid was collected and dried on a high vacuum for 24 hours to give 32 (23.1 g, 137 mmol, 95% yield) as an off-white solid. TLC: R_{f}=0.73 (20% EtOAc in hexanes, UV). ¹H NMR: (600 MHz, CDCl₃) δ 8.10 (d, J=5.2 Hz, 1H), 6.82 (d, J=5.5 Hz, 1H), 4.01 (s, 3H), 2.33 (s, 3H) ppm. ¹³C NMR: (151 MHz, CDCl₃) δ 155.2, 147.6, 141.6, 136.4, 118.9, 54.5, 16.9 ppm.

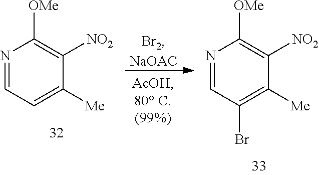

In a septum capped 500 mL round-bottomed flask equipped with a stir bar, addition funnel and argon balloon were added 32 (22.0 g, 131 mmol, 1 eq.), AcOH (131 mL) then NaOMe (38.7 g, 471 mmol, 3.6 eq.) to give a thick suspension. Br₂ (56.0 g, 18.1 mL, 351. mmol, 2.68 eq.) was added to the addition funnel where Br₂ was added dropwise over 30 minutes. After the addition of Br₂ the reaction mixture was heated to 80° C. for 14 hours. Cooled to 0° C. then a solution of 10% aqueous Na₂SO₄ (150 mL) was added followed by a saturated aqueous solution of Na₂SO₄ (150 mL). The resulting slurry stirred for 5 minutes then filtered through a sintered glass funnel. The solid collected was washed with water and dried under high vacuum to give 33 (32.0 g, 130 mmol, 99% yield) as a white solid. TLC: R_{f}=0.88 (20% EtOAc in hexanes, UV). ¹H NMR: (600 MHz, CDCl₃) δ 8.31 (s, 1H), 4.00 (s, 3H), 2.36 (s, 3H) ppm. ¹³C NMR: (151 MHz, CDCl₃) δ 154.2, 148.9, 140.9, 136.6, 114.6, 54.8, 17.8 ppm.

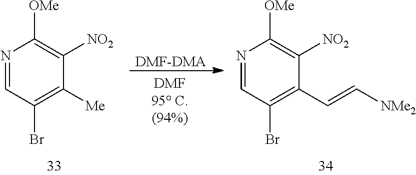

In a 1 L round-bottomed flask equipped with a stir bar and addition funnel was added 33 (47.0 g, 190 mmol, 1 eq.) and DMF (380 mL) then heated to 80° C. DMF-DMA (221 mL, 1.57 mol, 8.26 eq.) was added to the addition funnel then added dropwise to the reaction mixture while at 80° C. Once addition of DMF-DMA was complete, the reaction mixture was heated to 95° C. for 6 hours then cooled to room temperature and poured over ice-cold water (1 L). The resulting red slurry was filtered through a sintered glass funnel, rinsed with water and dried under vacuum to give 34 (54.2 g, 179 mmol, 94% yield) as a red solid. TLC: R_{f}=0.59 (20% EtOAc in hexanes, UV). ¹H NMR: (600 MHz, CDCl₃) δ 8.11 (s, 1H), 6.99 (d, J=13.6 Hz, 1H), 4.91 (d, J=13.6 Hz, 1H), 3.94 (s, 3H), 2.91 (s, 6H) ppm. ¹³C NMR: (151 MHz, CDCl₃) δ 154.7, 148.2, 147.2, 139.8, 131.4, 111.7, 87.6, 54.4, 40.7 ppm.

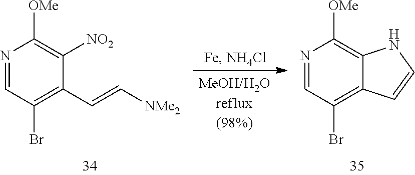

In a 1 L round-bottomed flask equipped with a stir bar and reflux condenser was added H₂O (100 mL), Fe (40.8 g, 729 mmol, 21 eq.) and NH₄Cl (16.7 g, 312 mmol, 9 eq.) followed by 34 (10.5 g, 34.8 mmol, 1 eq.) and MeOH (300 mL) then heated to 110° C. (reflux) for 24 hours. The reaction mixture was filtered through a pad of celite while still hot and rinsed with warm MeOH (3×50 mL), filtrate was concentrated, and H₂O (125 mL) was added. The resulting precipitate was filtered and washed with water (100 mL) and dried to give 35 (7.70 g, 33.9 mmol, 98% yield) as a beige solid. TLC: R_{f}=0.35 (20% EtOAc in hexanes, UV). ¹H NMR: (500 MHz, CDCl₃) δ 8.71 (s, 1H), 7.84 (s, 1H), 7.31 (dd, J=3.1, 2.5 Hz, 1H), 6.57 (dd, J=3.0, 2.3 Hz, 1H), 4.08 (s, 3H) ppm. ¹³C NMR: (126 MHz, CDCl₃) δ 150.4, 135.7, 134.3, 126.4, 120.9, 105.5, 103.3, 53.5 ppm.

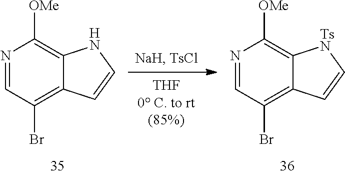 [SMILES: COc1ncc(Br)c2cc[nH]c12 ; COc1ncc(Br)c2ccn(S(=O)(=O)c3ccc(C)cc3)c12]

In a septum capped 500 mL round-bottomed flask equipped with a stir bar and argon balloon was added 35 (3.2 g, 14.1 mmol, 1 eq.) and THF (100 mL) then cooled to 0° C. NaH (846 mg, 21.1 mmol, 1.5 eq., 60% wt) was added portion wise then stirred at 0° C. for 15 minutes. Ts-Cl (3.22 g, 16.9 mmol, 1.2 eq.) was added in one portion then stirred at room temperature for 2 hours. The reaction was quenched with saturated aqueous NH₄Cl (120 mE), extracted aqueous layer with EtOAc (4×60 mE), combined organic layer dried over Na₂SO₄, filtered, concentrated and adsorbed to silica gel. Purification by silica gel column chromatography using hexanes/EtOAc (0% to 10% EtOAc gradient) provided 36 (4.55 g, 14.1 mmol, 85% yield) as light-yellow solid. TLC: R_{f}=0.53 (20% EtOAc in hexanes, UV). ¹H NMR: (600 MHz, CDCl₃) δ 7.97 (d, J=3.7 Hz, 1H), 7.89 (s, 1H), 7.77 (d, J=8.4 Hz, 2H), 7.27 (d, J=8.1 Hz, 2H), 6.67 (d, J=3.6 Hz, 1H), 3.88 (s, 3H), 2.38 (s, 3H) ppm. ¹³C NMR: (151 MHz, CDCl₃) δ 150.5, 145.3, 139.4, 139.2, 135.9, 131.2, 129.6, 127.9, 119.3, 106.1, 104.9, 53.3, 21.7 ppm.

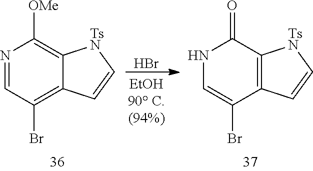 [SMILES: COc1ncc(Br)c2ccn(S(=O)(=O)c3ccc(C)cc3)c12 ; Br ; Cc1ccc(S(=O)(=O)n2ccc3c(Br)c[nH]c(=O)c32)cc1]

In a 250 mL round-bottomed flask equipped with a stir bar and reflux condenser was added 36 (7.8 g, 20.5 mmol, 1 eq.) and EtOH (32 mL). An aqueous solution of HBr (69.9 mL, 614 mmol, 48% wt, 30 eq.) was added and the reaction vial was capped tightly then heated to 90° C. for 2 hours. Cooled to 0° C. and filtered through a sintered glass funnel while rinsing with ice-cold water (100 mL). The collected solid was further dried under vacuum to give 37 (7.08 g, 19.3 mmol, 94% yield) as a white solid. TLC: R_{f}=0.39 (5% MeOH, in DCM, UV). ¹H NMR: (600 MHz, DMSO) δ 11.50 (s, 1H), 8.03 (d, J=3.5 Hz, 1H), 7.93 (d, J=8.4 Hz, 2H), 7.40 (d, J=8.1 Hz, 2H), 7.35 (s, 1H), 6.59 (d, J=3.5 Hz, 1H), 2.36 (s, 3H) ppm. ¹³C NMR: (151 MHz, DMSO) δ 152.5, 145.9, 137.2, 135.5, 131.6, 130.1, 130.1, 128.9, 122.3, 106.8, 91.8, 21.6 ppm.

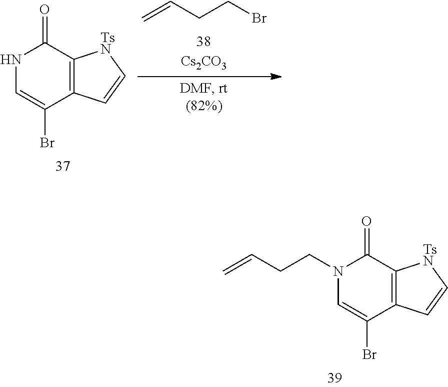

In a septum capped 30 mL reaction vial equipped with a stir bar was added 37 (1.05 g, 2.86 mmol, 1 eq.) and DMF (16 mL). Cs₂CO₃ (1.86 g, 5.72 mmol, 2 eq.) followed by 38 (0.377 mL, 0.502 g, 3.72 mmol, 1.3 eq.) then stirred at room temperature for 6 hours. The reaction mixture was filtered through a pad of celite while rinsing with EtOAc and the solvent removed under reduced pressure. The resulting crude oil was taken up in EtOAc (30 mL) and water (25 mL), aqueous layer extracted with EtOAc (4×30 mL), combined organic layer dried over Na₂SO₄, filtered and concentrated. Further purification by silica gel column chromatography using hexanes/EtOAc (0% to 20% EtOAc gradient) provided 39 (0.988 g, 2.34 mmol, 82% yield) as a light-yellow oil. TLC: R_{f}=0.71 (40% EtOAc in hexanes, UV). ¹H NMR: (600 MHz, CDCl₃) δ 7.98 (d, J=8.4 Hz, 2H), 7.91 (d, J=3.5 Hz, 1H), 7.28 (d, J=8.1 Hz, 2H), 7.12 (s, 1H), 6.49 (d, J=3.5 Hz, 1H), 5.78-5.63 (m, 1H), 5.00 (s, 1H), 4.99-4.96 (m, 1H), 3.94 (t, J=7.3 Hz, 2H), 2.43-2.39 (m, 2H), 2.39 (s, 3H) ppm. ¹³C NMR: (151 MHz, CDCl₃) δ 152.2, 145.1, 136.6, 135.8, 133.9, 131.9, 131.0, 129.5, 128.7, 122.6, 117.9, 106.1, 92.7, 48.8, 33.7, 21.7 ppm. HRMS: Calc'd for C₁₈H₁₈BrN₂O₃S [M+H⁺] 421.0216; found: 421.0219.

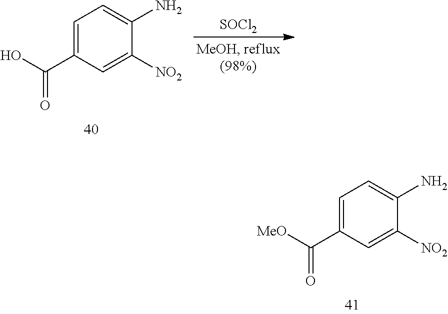

Methyl 4-amino-3-nitrobenzoate (41)¹⁵

In a 1 L round-bottomed flask equipped with a stir bar and reflux condenser was added 4-amino-3-nitrobenzoic acid (40) (50.0 g, 275 mmol, 1 eq.) and MeOH (600 mL). SOCl₂ (20.1 mL, 275 mmol, 1 eq.) was added over 15 minutes with vigorous stirring then heated to 93° C. (reflux) for 13 hours. Cooled to room temperature then chilled in an ice bath for 1 hour. The resulting suspension was filtered through a sintered glass funnel, solid collected and dried under vacuum to give 41 (52.8 g, 269 mmol, 98% yield) as a yellow solid that was use without further purification or characterization.

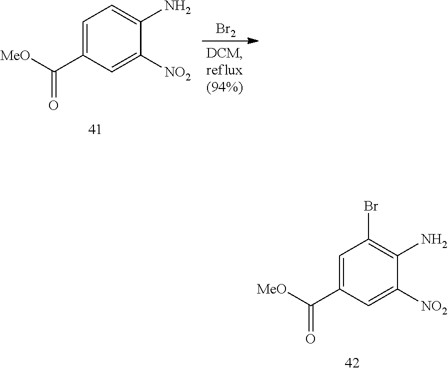

In a septum capped 1 L round-bottomed flask equipped with a stir bar, an aqueous 2M NaOH trap (to quench HBr generated) and argon balloon was added 41 (25.0 g, 127 mmol, 1 eq.) and DCM (690 mL). Br₂ (8.54 mL, 166 mmol, 1.3 eq.) was added then heated to 70° C. (reflux) for 2 hours. Another portion of Br₂ (3.28 mL, 63.8 mmol, 0.5 eq.) was added after cooling to room temperature then heated to 70° C. (reflux) for 1 hour to achieve full consumption of 41. Cooled to room temperature, quenched with a 10% aqueous solution of Na₂S₂O₃ (125 mL). Washed the organic layer with additional 10% aqueous solution of Na₂S₂O₃ (125 mL), water (125 mL), dried over MgSO₄, filtered and concentrated to give 42 (32.8 g, 119 mmol, 94% yield) as a yellow solid. TLC: R_{f}=0.54 (40% EtOAc in hexanes, UV). ¹H NMR: (500 MHz, CDCl₃) δ 8.83 (d, J=1.9 Hz, 1H), 8.34 (d, J=1.9 Hz, 1H), 7.00 (s, 1H), 3.91 (s, 3H) ppm. ¹³C NMR: (126 MHz, CDCl₃) δ 164.5, 144.8, 138.9, 131.9, 128.2, 118.7, 111.8, 52.5 ppm.

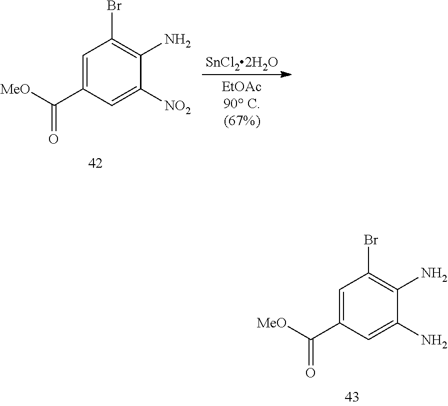

In a 500 mL round-bottomed flask equipped with a stir bar and reflux condenser was added 42(6.3 g, 22.9 mmol, 1 eq.) and EtOAc (191 mL). SnCl₂·2H₂0 (10.3 g, 45.8 mmol, 2 eq.) was added and heated to 90° C. for 18 hours. Additional SnCl₂·2H₂O (10.3 g, 45.8 mmol, 2 eq.) was added to achieve full consumption of 42 after heating to 90° C. for an additional 12 hours. Cooled to room temperature and quenched with saturated aqueous NaHCO₃ (200 mL), extracted aqueous layer with EtOAc (4×250 mL), combine organic layer dried over Na₂SO₄, filtered and concentrated to give 43 (3.75 g, 15.3 mmol, 67% yield) as a light-yellow solid. TLC: R_{f}=0.36 (40% EtOAc in hexanes, UV). ¹H NMR: (500 MHz, CDCl₃) δ 7.73 (d, J=1.8 Hz, 1H), 7.34 (d, J=1.8 Hz, 1H), 4.24 (s, 2H), 3.85 (s, 3H) ppm. ¹³C NMR: (126 MHz, CDCl₃) δ 166.3, 138.5, 133.6, 126.2, 121.2, 117.2, 109.1, 51.9 ppm.

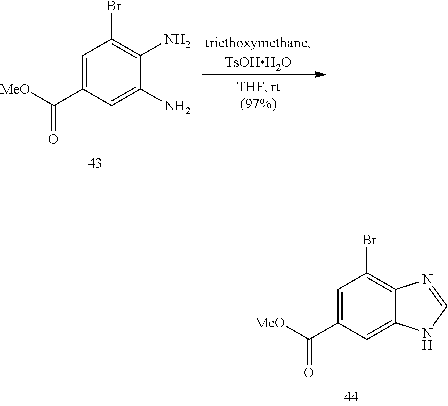 [SMILES: COC(=O)c1cc(N)c(N)c(Br)c1 ; "triethoxymethane,TsOH·H2O" ; COC(=O)c1cc(Br)c2nc[nH]c2c1]

In a 100 mL round-bottomed flask equipped with a stir bar was added 43 (0.814 g, 3.32 mmol, 1 eq.) and THF (29 mL). Triethoxymethane (1.11 mL, 6.64 mmol, 2 eq.) and p-TsOH (63.0 mg, 0.332 mmol, 0.1 eq.) was added, the flask was lightly capped and stirred at room temperature for 5 hours. The solvent was removed under reduced pressure, crude solid was taken up in EtOAc (60 mL) and water (45 mL), aqueous layer extracted with EtOAc (6×50 mL), combined organic layer dried over Na₂SO₄, filtered and concentrated to give 44 (0.821 g, 3.22 mmol, 97% yield) as a beige solid. TLC: R_{f}=0.26 (5% MeOH in DCM, UV). ¹H NMR: (500 MHz, DMSO) δ 8.53 (s, 1H), 8.19 (d, J=1.3 Hz, 1H), 7.97 (d, J=1.4 Hz, 1H), 3.89 (s, 3H) ppm. ¹³C NMR: (126 MHz, DMSO) δ 166.1, 146.0, 128.5, 125.9, 125.5, 125.3, 115.7, 52.8 ppm.

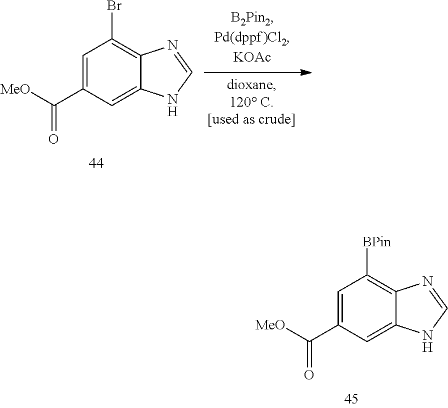

In a septum capped 100 mL round bottomed flask equipped with a stir bar and argon balloon was added 44 (0.820 g, 3.21 mmol, 1 eq.), B₂Pin₂ (1.14 g, 4.50 mmol, 1.4 eq.), Pd(dppf)Cl₂ (0.235 g, 0.321 mmol, 0.1 eq.), KOAc (0.628 g, 6.4 mmol, 2 eq.) and dioxane (30 mL). The resulting mixture was degassed using a flow of argon for 15 minutes then heated to 120° C. for 14 hours. Cooled to room temperature, diluted with EtOAc (30 mL) and filtered through a pad of celite while rinsing with EtOAc (50 mL). Solvent was removed then taken up in EtOAc (50 mL) and water (50 mL), aqueous layer extracted with EtOAc (4×50 mL), combine organic layer washed with brine (100 mL), dried over Na₂SO₄, filtered and concentrated. The black crude oil containing 45 was used without further purification or characterization.

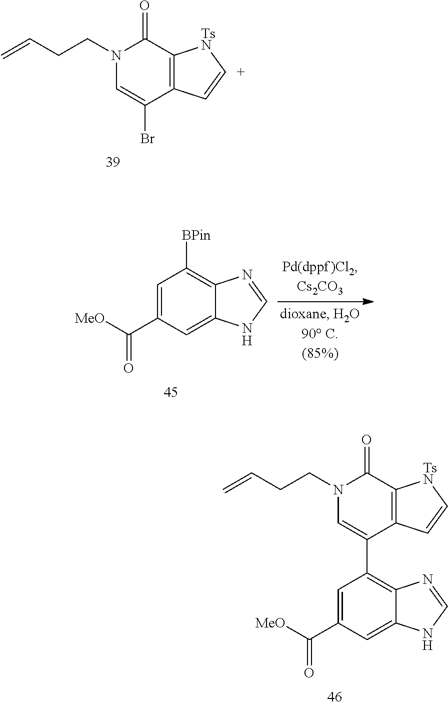

In a septum capped 50 mL round-bottomed flask equipped with a stir bar and argon balloon was 39 (0.158 g, 0.375 mmol, 1 eq.), 45 (0.170 g, 0.563 mmol, 1.5 eq.), Cs₂CO₃ (0.244 g, 0.750 mmol, 2 eq.), Pd(dppf)Cl₂ (27.0 mg, 0.038 mmol, 0.1 eq.), dioxane (9.5 mL) and water (2 mL). The resulting mixture was degassed using a flow of argon for 15 minutes then heated to 90° C. for 14 hours. Cooled to room temperature and the solvent was removed to give a crude oil that was taken up in EtOAc (30 mL) and water (20 mL). Aqueous layer extracted with EtOAc (4×20 mL), combined organic layers dried over Na₂SO₄, filtered and concentrated. Further purification by silica gel column chromatography using DCM/MeOH (0% to 4% MeOH gradient) provided 46 (0.125 g, 0.236 mmol, 85% yield) as a brown oil that solidified to a beige solid. TLC: R_{f}=0.37 (5% MeOH in DCM, UV). ¹H NMR: (500 MHz, CDCl₃) δ 8.45 (s, 1H), 8.25 (s, 1H), 7.97 (d, J=1.3 Hz, 1H), 7.92 (d, J=8.4 Hz, 2H), 7.87 (d, J=3.5 Hz, 1H), 7.30-7.26 (m, 2H), 7.25 (s, 1H), 6.42 (d, J=3.4 Hz, 1H), 5.65-5.51 (m, 1H), 4.87 (d, J=10.1 Hz, 1H), 4.82 (d, J=17.1 Hz, 1H), 3.95 (s, 3H), 3.74-3.54 (m, 2H), 2.38 (s, 3H), 2.30-2.21 (m, 2H) ppm. ¹³C NMR: (126 MHz, CDCl₃) δ 167.4, 152.4, 145.3, 143.3, 136.7, 135.8, 133.8, 132.3, 132.2, 131.7, 129.6, 128.3, 124.9, 124.5, 124.5, 122.5, 117.7, 111.4, 106.2, 52.3, 48.6, 33.6, 21.7 ppm.

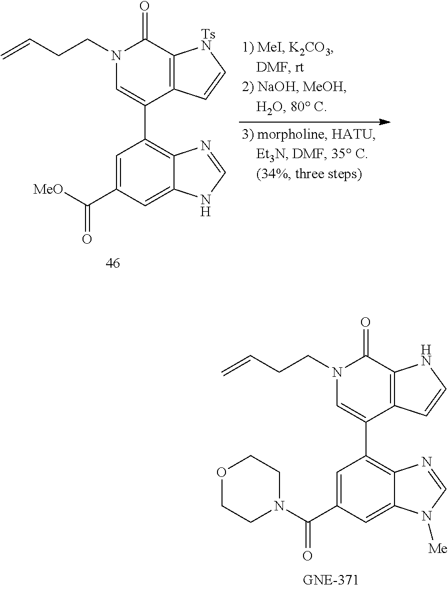 [SMILES: C=CCCn1cc(-c2cc(C(=O)OC)cc3[nH]cnc23)c2ccn(S(=O)(=O)c3ccc(C)cc3)c2c1=O ; C=CCCn1cc(-c2cc(C(=O)N3CCOCC3)cc3c2ncn3C)c2cc[nH]c2c1=O]

Step 1: In a 20 mL vial equipped with a stir bar was added 46 (0.143 g, 0.277 mmol, 1 eq.) and DMF (2 mE). K₂CO₃ (77.0 mg, 0.554 mmol, 2 eq.) was added followed by Mel (21 μL, 0.332 mmol, 1.2 eq.) then stirred at room temperature for 12 hours. Solvent was removed under reduced pressure and the crude residue was taken up in EtOAc (20 mL) and water (15 mL). Aqueous layer extracted with EtOAc (4×25 mL), combined organic layer dried over Na₂SO₄, filtered and concentrated to give the desired product (0.125 g, 0.236 mmol, 85% yield) as a brown oil containing approximately 15% of unwanted N-alkylated regioisomer (determined by LCMS) and used in the next step without further purification or characterization.

Step 2: In a 20 mL vial equipped with a stir bar was added the brown oil from Step 1 (0.149 g, 0.281 mmol, 1 eq.) and MeOH (4 mL). An aqueous solution of 1M NaOH (1.21 mL, 1.21 mmol, 4.3 eq.) was added and heated to 80° C. for 16 hours. The reaction was cooled to room temperature and the solvent removed under reduced pressure then taken up in water (4 mL), acidified to a pH of 5 using an aqueous solution of 2M HCl. The resulting solid was collected by filtration and washed with water to give the desired product (94.0 mg, 0.259 mmol, 92% yield) as a beige solid that was used in the next step without further purification or characterization.

Step 3: In a 2-dram reaction vial equipped with a stir bar was added the beige solid from Step 2 (0.094 mg, 0.259 mmol, 1 eq.) and DMF (2.2 mL). Et₃N (72.3 μL, 0.518 mmol, 2 eq.), HATU (0.197 g, 0.518 mmol, 2 eq.) and morpholine (44.8 μL, 0.518 mmol, 2 eq.) were added. The reaction was capped and heated to 35° C. for 8 hours. The solvent was removed, and the residue was purified by reverse phase chromatography using 19% ACN (0.1% NH₄0H) in water to provide GNE-371 (49 mg, 0.113 mmol, 44% yield) as a white solid. TLC: R_{f}=0.22 (5% MeOH in DCM, UV). ¹H NMR: (500 MHz, DMSO) δ 12.08 (s, 1H), 8.33 (s, 1H), 7.84 (s, 1H), 7.64 (d, J=1.3 Hz, 1H), 7.46 (d, J=1.3 Hz, 1H), 7.34 (s, 1H), 6.41-6.34 (m, 1H), 5.88 (ddt, J=16.9, 10.1, 6.7 Hz, 1H), 5.10 (dd, J=17.2, 1.7 Hz, 1H), 5.02 (dd, J=10.3, 1.7 Hz, 1H), 4.13 (t, J=7.2 Hz, 2H), 3.91 (s, 3H), 3.72-3.42 (m, 8H) ppm. ¹³C NMR: (126 MHz, DMSO) δ 170.1, 154.1, 146.3, 141.9, 135.6, 135.3, 130.3, 130.2, 129.3, 128.5, 127.4, 123.9, 120.2, 117.6, 111.4, 108.7, 103.4, 66.7, 60.0, 47.3, 34.2, 31.4 ppm. HRMS: Calc'd for C₂₄H₂₆N₅O₃ [M+H⁺] 423.2030; found: 423.2026.

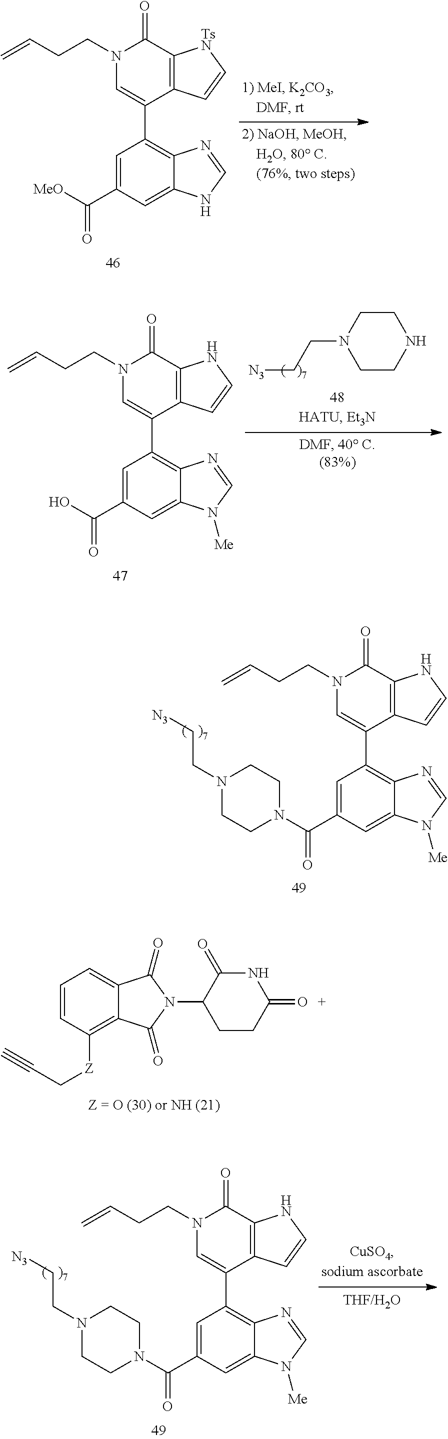

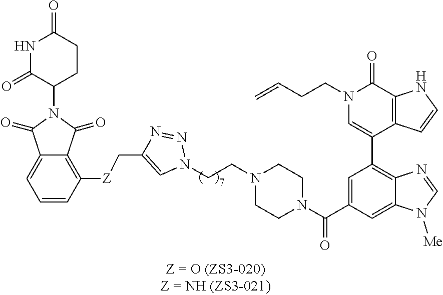

Synthetic Scheme of GNE371 PROTACs with a Western Exit Vector.

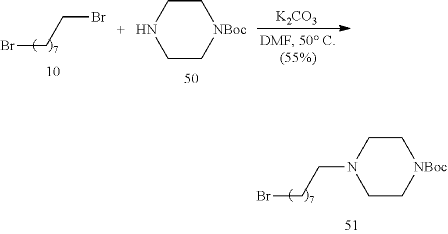

In a 20 mL septum capped vial equipped with a stir bar was 10 (1.02 g, 3.76 mmol, 1 eq.) in DMF (7 mL) at room temperature. 50 (702 mg, 3.76 mmol, 1 eq.) and K₂CO₃ (571 mg, 4.13 mmol, 1.1 eq.) were added, the vial capped and stirred at room temperature for 14 hours then heated to 50° C. for 4 hours. The reaction mixture was cooled to room temperature, quenched with water (50 mL), and extracted with EtOAc (4×50 mL). The combined organic layers were dried over Na₂SO₄, filtered and adsorbed to silica gel. Purification by silica gel column chromatography using hexanes/EtOAc (0% to 50% EtOAc) provided 51 (776 mg, 2.06 mmol, 55% yield) as a light-yellow oil. TLC: R_{f}=0.38 (50% EtOAc in hexanes, PMA stain). ¹H NMR: (500 MHz, CDCl₃) δ 3.43 (t, J=5.1 Hz, 4H), 3.39 (t, J=6.8 Hz, 2H), 2.37 (t, J=4.9 Hz, 4H), 2.36-2.29 (m, 2H), 1.89-1.79 (m, 2H), 1.52-1.46 (m, 2H), 1.45 (s, 9H), 1.44-1.38 (m, 2H), 1.30 (q, J=3.3, 2.5 Hz, 6H) ppm. ¹³C NMR: (126 MHz, CDCl₃) δ 154.75, 79.59, 58.75, 53.05, 34.00, 32.78, 29.32, 28.67, 28.44, 28.10, 27.40, 26.70 ppm.

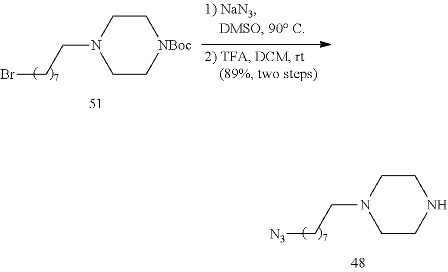

Step1: In a 20 mL capped vial equipped with a stir bar was 51 (538 mg, 1.43 mmol, 1 eq.) in DMSO (3 mL) at room temperature. NaN₃ (304 mg, 3.14 mmol, 2.2 eq.) was added, the vial capped and heated to 70° C. for 3 hours then to 90° C. for 14 hours. The reaction mixture was cooled to room temperature and diluted with EtOAc (80 mL) then partitioned with half-saturated aqueous NaCl (40 mL). The organic layer was further washed with half-saturated aqueous NaCl (2×40 mL), dried over Na₂SO₄, filtered and concentrated to give the desired alkyl azide (480 mg, 1.41 mmol, 99% yield) as an orange oil that was used in the next step without further purification. ¹H NMR: (500 MHz, CDCl₃) δ 3.43 (t, J=5.1 Hz, 4H), 3.24 (t, J=7.0 Hz, 2H), 2.37 (t, J=5.0 Hz, 4H), 2.34-2.29 (m, 2H), 1.62-1.55 (m, 2H), 1.52-1.46 (m, 2H), 1.44 (s, 9H), 1.38-1.27 (m, 8H) ppm. ¹³C NMR: (126 MHz, CDCl₃) δ 154.75, 79.59, 58.74, 53.04, 51.46, 29.36, 29.06, 28.81, 28.43, 27.40, 26.69, 26.65 ppm.

Step 2: In a 20 mL septum capped vial equipped with a stir bar and an argon balloon was the crude from step 1 (460 mg, 1.35 mmol, 1 eq.) in DCM (4 mL) at room temperature. TFA (1.56 mL, 20.3 mmol, 15 eq.) was added dropwise then the reaction stirred at room temperature for 30 minutes. The solvent and excess TFA was removed under reduced pressure and the crude taken up in DCM (100 mL). The organic layer was washed with saturated aqueous Na₂CO₃ (3×25 mL), dried over Na₂SO₄, filtered and concentrated to give 48 (292 mg, 1.22 mmol, 90% yield) as a light-yellow oil that was used in the next step without further purification. ¹H NMR: (500 MHz, CDCl₃) δ 4.75 (s, 1H), 3.25 (t, J=6.9 Hz, 2H), 3.10 (t, J=5.0 Hz, 4H), 2.62 (t, J=5.3 Hz, 4H), 2.41-2.35 (m, 2H), 1.60-1.56 (m, 2H), 1.49-1.43 (m, 2H), 1.37-1.29 (m, 8H) ppm. ¹³C NMR: (126 MHz, CDCl₃) δ 58.41, 51.45, 51.15, 44.19, 29.29, 29.05, 28.81, 27.19, 26.64, 26.38 ppm. HRMS: Calc'd for C₁₂H₂₆N₅ [M+H⁺] 240.2183; found: 240.2184.

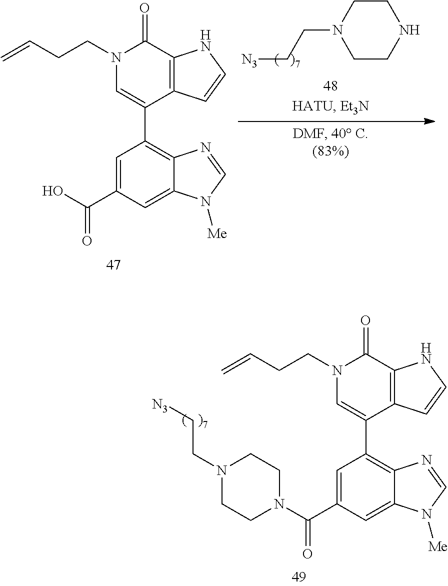

47 was obtained from Step 2 of the final three step sequence for the synthesis of GNE-371.

In a 1-dram capped vial equipped with a stir bar was 47 (35.0 mg, 96.6 μmol, 1 eq.), 48 (34.7 mg, 145 μmol, 1.5 eq.) and DMF (1 mL) at room temperature. Et₃N (27 μL, 193 mol, 2 eq.) and HATU (73.5 mg, 193 μmol, 2 eq.) were added and the reaction mixture was heated to 40° C. for 15 hours then cooled to room temperature. The solvent was removed under reduced pressure and the crude was taken up in EtOAc (10 mL) and portioned with saturated aqueous NH₄Cl (6 mL). The aqueous layer was extracted with EtOAc (4×10 mL), combined organic layers were dried over Na₂SO₄, filtered and concentrated. Further purification by silica gel column chromatography using DCM/MeOH (0% to 8% MeOH) provided 49 (47.0 mg, 80.5 μmol, 83% yield) as a beige oil. ¹H NMR: (500 MHz, CDCl₃) δ 10.18 (s, 1H), 7.98 (s, 1H), 7.63 (s, 1H), 7.54 (d, J=15.4 Hz, 2H), 7.28-7.26 (m, 1H), 6.47 (s, 1H), 5.87 (ddt, J=17.0, 10.2, 6.8 Hz, 1H), 5.14-5.04 (m, 2H), 4.22 (t, J=7.4 Hz, 2H), 4.13-3.71 (m, 7H), 3.25 (t, J=6.9 Hz, 2H), 2.61 (dt, J=14.4, 7.6 Hz, 6H), 2.26-2.19 (m, 1H), 2.01 (q, J=6.1 Hz, 1H), 1.57 (d, J=7.3 Hz, 2H), 1.37-1.32 (m, 10H) ppm. ¹³C NMR: (126 MHz, CDCl₃) δ 170.75, 154.51, 144.88, 135.16, 134.52, 130.04, 129.78, 129.75, 129.65, 126.47, 124.23, 120.66, 117.47, 113.93, 112.46, 108.07, 103.80, 58.28, 51.42, 48.43, 35.96, 34.17, 31.92, 31.40, 29.71, 28.78, 26.60, 25.55, 22.70, 14.13 ppm. HRMS: Calc'd for C₃₂H₄₂N₉O₂ [M+H⁺] 584.3456; found: 584.3455.

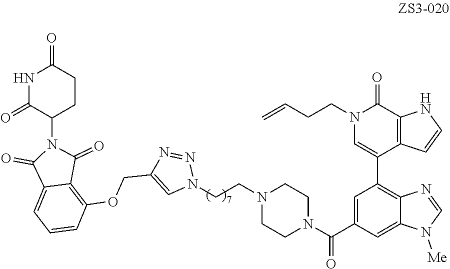

The title compound was prepared using GP-2.

49 (19.0 32.6 μmol, 1 eq.), 30 (10.2 mg, 32.6 μmol, 1 eq.), sodium ascorbate (1.3 mg, 6.5 μmol, 0.2 eq.), CuSO₄ (1.0 mg, 6.5 μmol, 0.2 eq.), THF (0.7 mL) and H₂O (20 μL) were used. After 18 hours at room temperature, another 0.2 equivalents of sodium ascorbate and CuSO₄ were added to achieve further consumption of the starting materials. Purification by silica gel column chromatography using DCM/MeOH (0% to 10% MeOH) provided ZS3-020 (15.0, 16.7 μmol, 51% yield) as a beige solid. ¹H NMR: (500 MHz, DMSO) δ 12.09 (s, 1H), 11.11 (s, 1H), 8.27 (s, 1H), 7.88-7.80 (m, 2H), 7.73 (d, J=8.6 Hz, 1H), 7.61 (s, 1H), 7.48 (d, J=7.2 Hz, 1H), 7.44 (s, 1H), 7.35 (t, J=2.6 Hz, 1H), 7.09 (d, J=6.9 Hz, 1H), 6.83 (d, J=7.7 Hz, 1H), 6.37 (t, J=2.1 Hz, 1H), 5.88 (ddt, J=17.0, 10.2, 6.7 Hz, 1H), 5.42 (s, 2H), 5.13-5.01 (m, 3H), 4.37 (t, J=7.0 Hz, 2H), 4.14 (t, J=7.2 Hz, 2H), 3.91 (s, 3H), 3.69-3.50 (m, 5H), 2.93-2.82 (m, 1H), 2.61-2.55 (m, 1H), 2.45-2.23 (m, 4H), 2.06-1.97 (m, 1H), 1.84-1.75 (m, 2H), 1.57 (s, 1H), 1.43 (s, 2H), 1.22 (d, J=17.8 Hz, 10H) ppm. HRMS: Calc'd for C₄₈H₅₄N₁₁O₇ [M+H⁺] 896.4202; found: 896.4198.

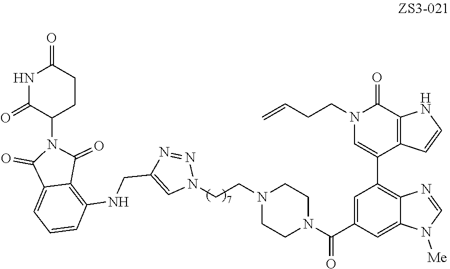

The title compound was prepared using GP-2.

49 (18.0 30.8 μmol, 1 eq.), 21 (9.6 mg, 30.8 μmol, 1 eq.), sodium ascorbate (1.2 mg, 6.2 μmol, 0.2 eq.), CuSO₄ (1.0 mg, 6.2 μmol, 0.2 eq.), THF (0.7 mL) and H₂O (20 μL) were used. After 18 hours at room temperature, another 0.2 equivalents of sodium ascorbate and CuSO₄ were added to achieve further consumption of the starting materials. Purification by silica gel column chromatography using DCM/MeOH/NH₄OH (10% MeOH/0.2% NH₄0H) provided ZS3-021 (17.0, 18.9 μmol, 62% yield) as a yellow solid. ¹H NMR: (500 MHz, CDCl₃) δ 10.39 (s, 1H), 9.70 (s, 1H), 7.97 (s, 1H), 7.64 (s, 1H), 7.56-7.52 (m, 2H), 7.51-7.45 (m, 2H), 7.13 (d, J=7.2 Hz, 1H), 6.99 (d, J=8.5 Hz, 1H), 6.64 (t, J=6.0 Hz, 1H), 6.45 (t, J=2.6 Hz, 1H), 5.93-5.81 (m, 1H), 5.15-5.01 (m, 2H), 4.92 (dd, J=12.2, 5.4 Hz, 1H), 4.62 (d, J=5.9 Hz, 2H), 4.32 (td, J=7.0, 2.8 Hz, 2H), 4.21 (t, J=7.4 Hz, 2H), 3.91 (s, 3H), 3.84-3.53 (m, 4H), 2.85-2.70 (m, 3H), 2.60 (q, J=7.2 Hz, 5H), 2.11 (ddd, J=16.5, 10.5, 7.9 Hz, 1H), 1.87 (t, J=7.2 Hz, 2H), 1.73 (s, 2H), 1.52 (s, 2H), 1.36-1.16 (m, 1OH) ppm. HRMS: Calc'd for C₄₈H₅₅N₂O₆ [M+H⁺] 895.4362; found: 895.4362.

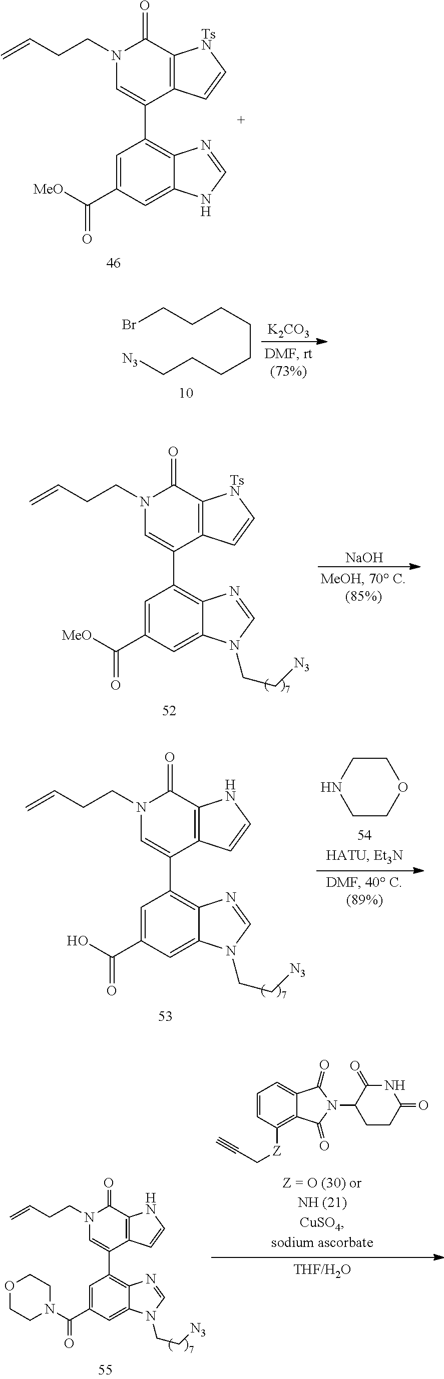

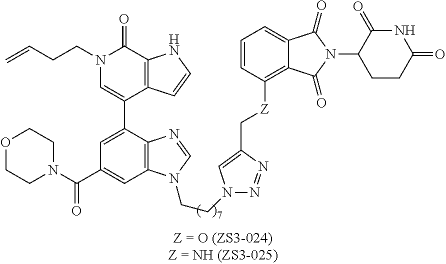

Synthetic Scheme of GNE371 PROTACs with an Eastern Exit Vector.

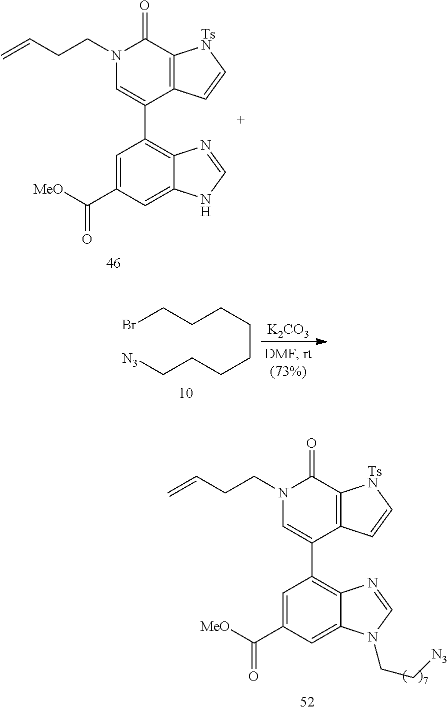

In a 20 mL septum capped vial equipped with a stir bar and argon balloon was 46 (132 mg, 0.256 mmol, 1 eq.) and DMF (2.5 mL) at room temperature. The reaction mixture stirred at room temperature for 10 minutes then 10 (71.8 mg, 0.307 mmol, 1.2 eq.) was added and stirred at room temperature for 13 hours. EtOAc (20 mL) was added and filtered through a pad of Celite while rinsing with EtOAc. The filtrate was concentrated to give a crude oil that was purified by silica gel column chromatography using DCM/MeOH (0% to 5% MeOH) to give 52 (125 mg, 0.187 mmol, 73% yield) as a beige oil. ¹H NMR: (500 MHz, CDCl₃) δ 8.15 (d, J=1.4 Hz, 1H), 8.09 (s, 1H), 8.07 (d, J=1.4 Hz, 1H), 8.01 (d, J=8.4 Hz, 2H), 7.91 (d, J=3.5 Hz, 1H), 7.55 (s, 1H), 7.29 (d, J=8.1 Hz, 2H), 6.53 (d, J=3.5 Hz, 1H), 5.78 (ddt, J=17.1, 10.2, 6.8 Hz, 1H), 5.07-4.94 (m, 2H), 4.27 (t, J=7.2 Hz, 2H), 4.09 (t, J=7.5 Hz, 2H), 3.99 (s, 3H), 3.25 (t, J=6.9 Hz, 2H), 2.49 (q, J=7.0 Hz, 2H), 2.41 (s, 3H), 1.60-1.54 (m, 2H), 1.42-1.31 (m, 10H) ppm. HRMS: Calc'd for C₃₅H₄₀N₇O₅S [M+H⁺] 670.2806; found: 670.2800.

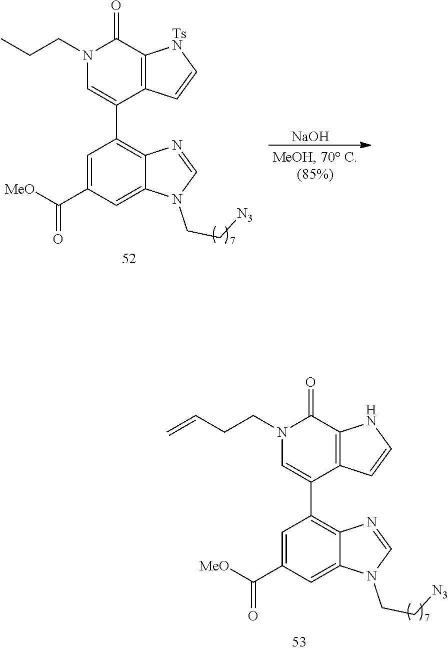

In a 2-dram capped reaction vial equipped with a stir bar was 52 (118 mg, 0.176 mmol, 1 eq.) in MeOH (1.8 mL) at 70° C. 1 M NaOH (0.758 mL, 0.758 mmol, 4.3 eq.) was added dropwise to the heated solution while stirring then continued heating for an additional 3 hours. The reaction mixture was cooled to room temperature and the solvent removed under reduced pressure. H₂O (8 mL) was added and washed with EtOAc (3×8 mL). The pH of the aqueous layer was adjusted to a pH of 3 with 1 M HCl and back extracted with EtOAc (3×15 mL). The combined organic layers from the back extraction were dried over Na₂SO₄, filtered and concentrated to give 53 (73 mg, 0.146 mmol, 83% yield) as light-beige oil that solidified upon standing. The crude material was sufficiently pure by LCMS and was used in the next step without further purification. ¹H NMR: (500 MHz, DMSO) δ 12.84 (s, 1H), 12.11 (s, 1H), 8.48 (s, 1H), 8.17 (d, J=1.5 Hz, 1H), 8.15-8.12 (m, 1H), 7.94 (s, 1H), 7.37 (t, J=2.7 Hz, 1H), 6.42 (t, J=2.3 Hz, 1H), 5.89 (ddt, J=17.0, 10.2, 6.7 Hz, 1H), 5.15-4.98 (m, 2H), 4.37 (t, J=7.1 Hz, 2H), 4.15 (t, J=7.3 Hz, 2H), 3.30-3.27 (m, 2H), 1.88-1.80 (m, 2H), 1.54-1.47 (m, 2H), 1.39-1.14 (m, 10H) ppm. HRMS: Calc'd for C₂₇H₃₂N₇O₃ [M+H⁺] 502.2561; found: 502.2559.

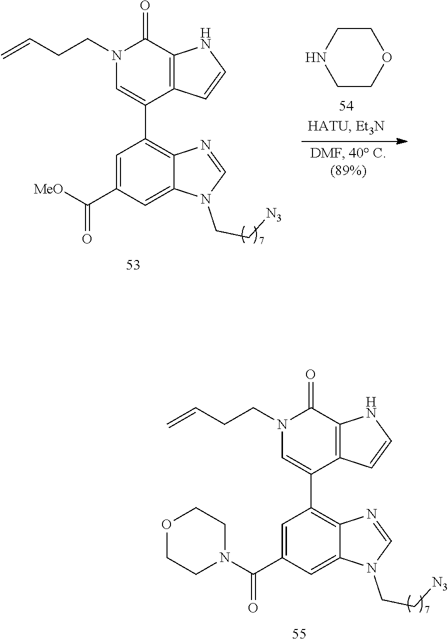

In a 2-dram capped reaction vial equipped with a stir bar was 53 (66 mg, 0.132 mmol, 1 eq.) in DMF (1.3 mL) at room temperature. 54 (22.7 μL, 0.263 mmol, 2 eq.), HATU (100 mg, 0.263 mmol, 2 eq.) and Et₃N (36.7 μL, 0.263 mmol, 2 eq.) were added then heated to 40° C. for 72 hours. The reaction mixture was cooled to room temperature and the solvent was removed under reduced pressure. The crude reaction mixture was directly adsorbed to silica gel and purified by silica gel column chromatography using DCM/MeOH (0% to 5% MeOH) to provide 55 (67 mg, 0.117 mmol, 89% yield) as a beige oil. ¹H NMR: (500 MHz, CDCl₃) δ 11.13 (s, 1H), 8.09 (s, 1H), 7.60 (s, 1H), 7.54 (d, J=8.6 Hz, 2H), 7.30-7.27 (m, 1H), 6.42 (s, 1H), 5.85 (ddt, J=17.0, 10.3, 6.8 Hz, 1H), 5.07 (dd, J=28.6, 13.7 Hz, 2H), 4.27-4.18 (m, 4H), 3.80-3.66 (m, 6H), 3.25 (t, J=6.9 Hz, 2H), 2.64-2.55 (m, 2H), 1.98-1.88 (m, 2H), 1.57 (q, J=7.1 Hz, 2H), 1.42-1.28 (m, 1OH) ppm. ¹³C NMR: (126 MHz, CDCl₃) δ 170.59, 154.52, 144.14, 139.97, 134.40, 134.20, 130.61, 130.23, 129.36, 128.74, 127.37, 123.97, 120.13, 117.53, 112.76, 108.69, 103.29, 66.93, 51.38, 48.65, 46.02, 45.71, 34.12, 31.03, 29.79, 28.96, 28.76, 26.77, 26.59, 8.63 ppm. HRMS: Calc'd for C₃₁H₃₉N₈O₃ [M+H⁺] 571.3140; found: 571.3144.

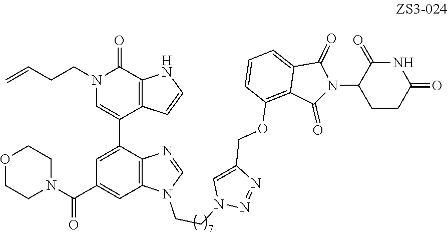

The title compound was prepared using GP-2.

55 (25.0 mg, 43.8 μmol, 1 eq.), 30 (14.0 mg, 43.8 μmol, 1 eq.), sodium ascorbate (1.7 mg, 8.8 μmol, 0.2 eq.), CuSO₄ (1.4 mg, 8.8 μmol, 0.2 eq.), THF (0.9 mL) and H₂O (20 L) were used. After 1.5 hours at room temperature, another 0.2 equivalents of sodium ascorbate and CuSO₄ were added along with 0.2 equivalents of 30 to achieve further consumption of 55. Purification by preparative TLC using 8% MeOH in DCM provided ZS3-024 (22.4 mg, 25.4 μmol, 58% yield) as a beige foam. ¹H NMR: (500 MHz, CDCl₃) δ 10.94 (s, 1H), 9.26 (s, 1H), 8.14 (s, 1H), 7.73 (s, 1H), 7.66 (dd, J=8.4, 7.4 Hz, 1H), 7.60 (s, 1H), 7.56-7.51 (m, 2H), 7.48-7.44 (m, 2H), 7.23 (s, 1H), 6.40 (s, 1H), 5.85 (ddt, J=17.1, 10.2, 6.8 Hz, 1H), 5.44 (s, 2H), 5.13-5.00 (m, 2H), 4.94 (dd, J=12.2, 5.3 Hz, 1H), 4.32 (t, J=7.1 Hz, 2H), 4.24-4.16 (m, 4H), 3.79-3.66 (m, 5H), 2.88-2.70 (m, 3H), 2.59 (q, J=7.1 Hz, 2H), 2.24-2.19 (m, 1H), 2.12-2.07 (m, 1H), 1.91-1.83 (m, 4H), 1.33-1.27 (m, 1OH) ppm. HRMS: Calc'd for C₄₇H₅₁N₁₀O₈ [M+H⁺] 883.3886; found: 883.3883.

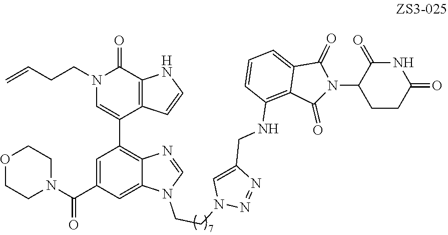

The title compound was prepared using GP-2.

55 (25.0 mg, 43.8 μmol, 1 eq.), 21 (14 mg, 43.8 μmol, 1 eq.), sodium ascorbate (1.7 mg, 8.8 μmol, 0.2 eq.), CuSO₄ (1.4 mg, 8.8 μmol, 0.2 eq.), THF (0.9 mL) and H₂O (20 μL) were used. After 1.5 hours at room temperature, another 0.2 equivalents of sodium ascorbate and CuSO₄ were added along with 0.2 equivalents of 21 to achieve further consumption of 55. Purification by preparative TLC using 8% MeOH in DCM provided ZS3-024 (21.8 mg, 24.7 μmol, 56% yield) as a yellow solid. ¹H NMR: (500 MHz, CDCl₃) δ 10.88 (s, 1H), 9.13 (s, 1H), 8.23 (s, 1H), 7.57 (d, J=15.5 Hz, 3H), 7.49-7.43 (m, 2H), 7.22 (s, 1H), 7.11 (d, J=7.1 Hz, 1H), 6.98 (d, J=8.5 Hz, 1H), 6.69 (t, J=5.8 Hz, 1H), 6.39 (s, 1H), 5.84 (ddt, J=17.0, 10.2, 6.8 Hz, 1H), 5.13-5.00 (m, 2H), 4.90 (dd, J=12.2, 5.3 Hz, 1H), 4.62 (d, J=5.9 Hz, 2H), 4.29 (t, J=7.1 Hz, 2H), 4.20 (dt, J=24.5, 7.2 Hz, 4H), 3.79-3.64 (m, 5H), 2.89-2.69 (m, 3H), 2.58 (q, J=7.1 Hz, 2H), 2.22 (dd, J=14.1, 6.5 Hz, 1H), 2.13-2.07 (m, 1H), 1.86 (dq, J=21.2, 7.0, 6.5 Hz, 4H), 1.28 (s, 1OH) ppm. HRMS: Calc'd for C₄₇H₅₂N₁₁O₇ [M+H⁺] 882.4046; found: 882.4042.

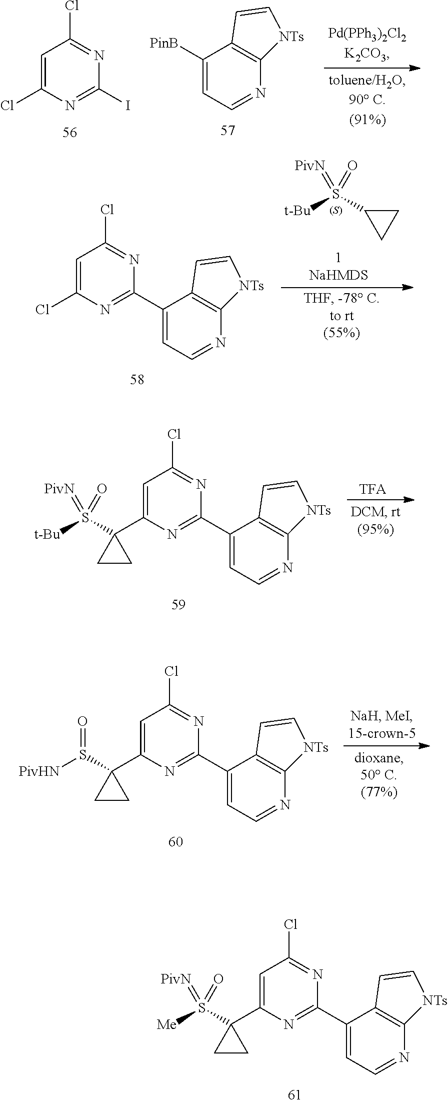

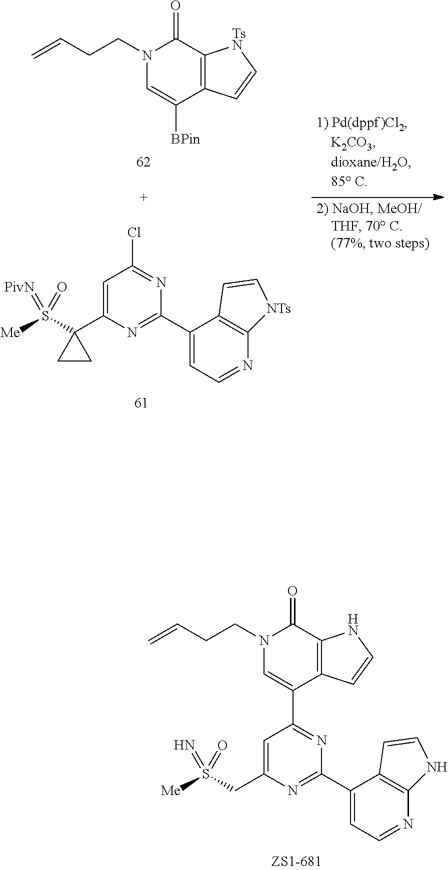

### Synthetic Scheme for the Synthesis of ZS1-681.

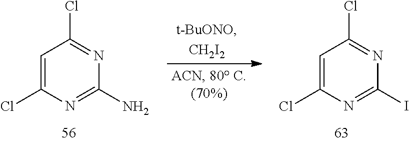 [SMILES: Nc1nc(Cl)cc(Cl)n1 ; Clc1cc(Cl)nc(I)n1]

The title compound was prepared according to a known literature procedure.¹⁶

In a 500 mL round-bottomed flask equipped with a stir bar, septum capped reflux condenser and argon balloon were added 56 (10 g, 61 mmol, 1 eq.), ACN (86 mL) and CH₂I₂(5.41 mL, 67.1 mmol, 1.1 eq.). To the stirring solution, t-BuONO (40.3 mL, 305 mmol, 5 eq., 90%) was added via syringe and resulting reaction mixture was heated to 80° C. for 4 hours. The reaction was cooled to room temperature and the solvents were removed under vacuum. The crude residue was taken up in EtOAc (450 mL) and washed with saturated aqueous Na₂SO₃ (3×150 mL). The combined aqueous layers were back extracted with EtOAc (2×150 mL) and combined with the first organic layer. The combined organic layers were dried over Na₂SO₄ then filtered and adsorbed to silica gel. Further purification by silica gel column chromatography using hexanes and EtOAc (0% to 10% EtOAc) provided 63 (11.7 g, 42.7 mmol, 70% yield) as an off-white solid. TLC: R_{f}=0.58 (10% EtOAc in hexanes)¹H NMR: (500 MHz, CDCl₃) δ 7.39 (s, 1H) ppm. ¹³C NMR: (126 MHz, CDCl₃) δ 161.04, 125.97, 120.87 ppm.

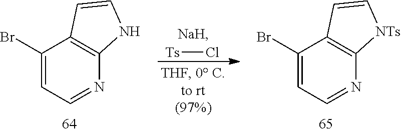 [SMILES: Brc1ccnc2[nH]ccc12 ; Cc1ccc(S(=O)(=O)Cl)cc1 ; Cc1ccc(S(=O)(=O)n2ccc3c(Br)ccnc32)cc1]

In a flame dried 500 mL septum capped round-bottomed flask equipped with a stir bar were added 64 (6.20 g, 31.5 mmol, 1 eq.) and THF (157 mL) then cooled to 0° C. NaH (1.51 g, 37.8 mmol, 1.2 eq.) was added portion-wise and stirred at 0° C. for 30 minutes followed by the addition of Ts-Cl (6.60 g, 34.6 mmol, 1.1 eq.) in one portion. The reaction mixture was stirred at 0° C. for 1 hour then warmed to room temperature where it stirred for 3 hours. The reaction was quenched with saturated aqueous NH₄Cl (125 mL), extracted with EtOAc (3×150 mL), dried over Na₂SO₄, filtered and concentrated. The crude solid was adsorbed to silica gel and filtered through a short plug of silica gel rinsing with hexanes (200 mL) then elution with (20% EtOAc in hexanes) to give 65 (10.7 g, 30.5 mmol, 97% yield) as a light-yellow solid. TLC: R_{f}=0.29 (10% EtOAc in hexanes)¹H NMR: (500 MHz, CDCl₃) δ 8.22 (d, J=5.3 Hz, 1H), 8.06 (d, J=8.4 Hz, 2H), 7.78 (d, J=4.0 Hz, 1H), 7.35 (d, J=5.2 Hz, 1H), 7.27 (d, J=8.7 Hz, 2H), 6.63 (d, J=4.1 Hz, 1H), 2.37 (s, 3H) ppm. ¹³C NMR: (126 MHz, CDCl₃) δ 146.73, 145.53, 145.02, 135.06, 129.74, 128.14, 126.96, 125.70, 124.35, 122.05, 104.86, 21.68 ppm.

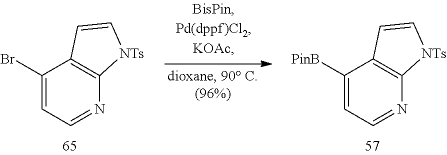

In a 250 mL septum capped round-bottomed flask equipped with a stir bar were added 65 (5.04 g, 14.4 mmol, 1 eq.), BisPin (5.10 g, 20.1 mmol, 1.4 eq.) and dioxane (72 mE). Pd(dppf)Cl₂ (0.525 g, 0.718 mmol, 0.05 eq.) and KOAc (2.82 g, 28.7 mmol, 2 eq.) were added and the reaction was heated to 90° C. for 52 hours. The reaction was cooled to room temperature and filtered through a pad of Celite in a sintered glass funnel. The filtrate was concentrated and adsorbed to silica gel. Purification by silica gel column chromatography using hexanes/EtOAc (0% to 10% EtOAc) provided 57(5.51 g, 13.8 mmol, 96% yield) as a white solid. TLC: R_{f}=0.18 (5% MeOH in DCM)*57 decomposes on TLC to boronic acid ¹H NMR: (500 MHz, CDCl₃) δ 8.42 (d, J=4.7 Hz, 1H), 8.02 (d, J=8.4 Hz, 2H), 7.74 (d, J=4.0 Hz, 1H), 7.52 (d, J=4.7 Hz, 1H), 7.23 (d, J=8.1 Hz, 2H), 7.01 (d, J=4.0 Hz, 1H), 2.33 (s, 3H), 1.35 (s, 12H) ppm. ¹³C NMR: (126 MHz, CDCl₃) δ 146.91, 145.00, 144.08, 135.53, 129.58, 128.01, 127.89, 127.37, 126.76, 124.56, 107.36, 84.41, 24.95, 21.61 ppm.

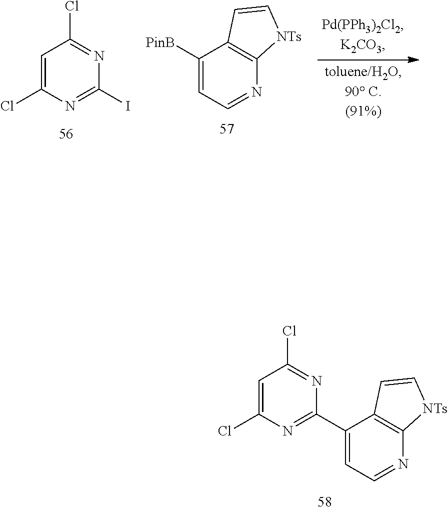

In a 20 mL septum capped vial equipped with a stir bar and argon balloon were added 56 (0.450 g, 1.64 mmol, 1 eq.), 57 (0.652 g, 1.64 mmol, 1 eq.) and toluene/water (13.5 mL/0.135 mL, 100:1). K₂CO₃ and Pd(PPh₃)₂Cl₂ were added and the reaction mixture was heated to 90° C. for 34 hours. The reaction was cooled to room temperature and diluted with EtOAc (50 mL) and water (15 mL). The aqueous layer was extracted with EtOAc (4×25 mL) and DCM (2×25 mL). The combined organic layers were dried over Na₂SO₄, filtered and adsorbed to silica gel. Purification by silica gel column chromatography using hexanes/DCM (10% to 70% DCM in hexanes) provided 58 (0.625 g, 1.49 mmol, 91% yield) as a white solid. TLC: R_{f}=0.516 (66% DCM in hexanes). ¹H NMR: (500 MHz, CDCl₃) δ 8.56 (d, J=5.1 Hz, 1H), 8.19 (d, J=5.1 Hz, 1H), 8.08 (d, J=8.5 Hz, 2H), 7.89 (d, J=4.0 Hz, 1H), 7.59 (d, J=4.0 Hz, 1H), 7.37 (s, 1H), 7.30-7.26 (m, 2H), 2.37 (s, 3H) ppm. ¹³C NMR: (126 MHz, CDCl₃) δ 164.42, 162.16, 148.72, 145.34, 144.89, 135.27, 134.88, 129.67, 128.24, 128.15, 121.35, 120.02, 118.37, 106.78, 21.66 ppm. HRMS: Calc'd for C₁₈H₁₂Cl₂N₄O₂SNa [M+Na⁺] 440.9950; found: 440.9958.

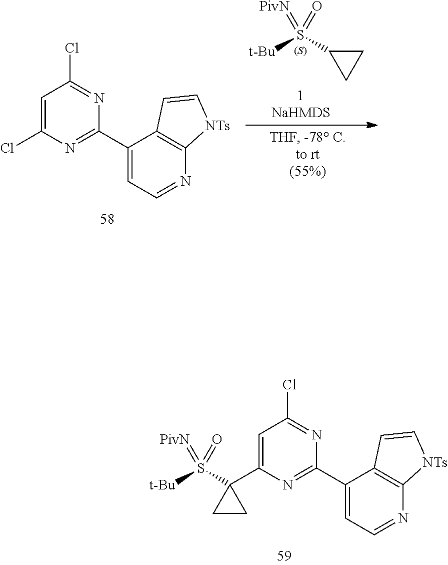

In a flame dried 500 mL septum capped round-bottomed flask equipped with a stir bar and argon balloon were added 58 (1.90 g, 4.53 mmol, 1 eq.), 1 (1.11 g, 4.53 mmol, 1 eq.) and THF (181 mL) then cooled to −78° C. NaHMDS (2.72 mL, 5.44 mmol, 1.2 eq., 2M in THF) was added via syringe and stirred at −78° C. for 10 hours. Additional NaHMDS (0.60 mL, 1.2 mmol, 0.26 eq., 2M in THF) was added and continued to stir at −78° C. for 2 hours. The reaction was quenched with saturated aqueous NH₄Cl (100 mL) while still at −78° C. The aqueous layer was extracted with EtOAc (3×100 mL), combined organic layers were dried over Na₂SO₄, filtered and concentrated. The crude residue was adsorbed to silica gel and further purified by silica gel column chromatography using hexanes/EtOAc (0% to 20% EtOAc in hexanes) to give 59 (1.56 g, 2.48 mmol, 55% yield) as light-yellow waxy solid. TLC: R_{f}=0.54 (40% EtOAc in hexanes). ¹H NMR: (500 MHz, CDCl₃) δ 8.58 (d, J=5.1 Hz, 1H), 8.20 (d, J=5.1 Hz, 1H), 8.09 (d, J=8.4 Hz, 2H), 7.90 (d, J=4.0 Hz, 1H), 7.72 (s, 1H), 7.58 (d, J=4.0 Hz, 1H), 7.28 (dd, J=8.6, 0.8 Hz, 2H), 2.57 (ddd, J=10.6, 8.1, 6.0 Hz, 1H), 2.37 (s, 3H), 2.14-2.06 (m, 1H), 1.98 (ddd, J=10.5, 7.8, 5.6 Hz, 1H), 1.42-1.40 (m, 1H), 1.38 (s, 9H), 1.25 (s, 9H) ppm. ¹³C NMR: (126 MHz, CDCl₃) δ 188.09, 166.14, 163.75, 162.20, 148.73, 145.40, 144.94, 135.66, 135.22, 129.69, 128.19, 128.13, 123.22, 121.23, 118.12, 106.64, 66.72, 42.29, 27.78, 24.96, 24.65, 21.69, 16.78, 14.73 ppm. HRMS: Calc'd for C₂₆H₂₇ClN₅O₄S₂ [M−t-Bu+H⁺] 572.1187; found: 572.1188.

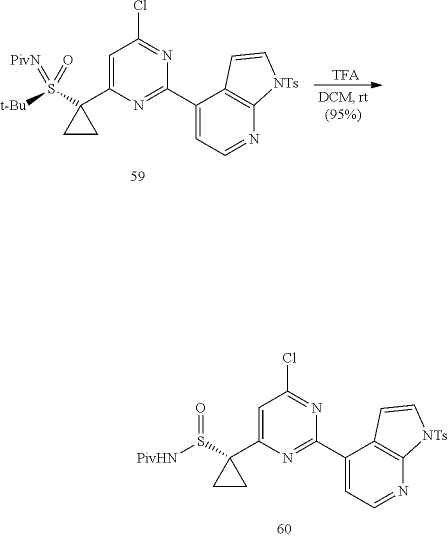

In a 40 mL septum capped reaction vial equipped with a stir bar and argon balloon were added 59 (1.52 g, 2.42 mmol, 1 eq.) and DCM (24 mL). TFA (0.205 mL, 2.66 mmol, 1.1 eq.) was added and the reaction stirred at room temperature for 4 hours. The reaction was quenched with saturated aqueous NaHCO₃ (40 mL) and the aqueous layer was extracted with DCM (3×40 mL), combined organic layers were dried over Na₂SO₄, filtered and concentrated. Further purification by silica gel column chromatography using hexanes/EtOAc (10% to 50% EtOAc in hexanes) to give 60 (1.31 g, 2.29 mmol, 95% yield) as an opaque oil. TLC: R_{f}=0.16 (40% EtOAc in hexanes). ¹H NMR: (500 MHz, CDCl₃) δ 8.57 (d, J=5.1 Hz, 1H), 8.30 (s, 1H), 8.13 (d, J=5.1 Hz, 1H), 8.11-8.05 (m, 2H), 7.89 (d, J=4.0 Hz, 1H), 7.50 (d, J=4.0 Hz, 1H), 7.34 (s, 1H), 7.28 (dd, J=8.7, 0.7 Hz, 2H), 2.37 (s, 3H), 1.92 (ddd, J=10.2, 7.2, 5.3 Hz, 1H), 1.72 (ddd, J=9.4, 7.2, 5.3 Hz, 1H), 1.65 (ddd, J=10.3, 7.2, 5.5 Hz, 1H), 1.51 (ddd, J=9.5, 7.2, 5.4 Hz, 1H), 0.99 (s, 9H) ppm. ¹³C NMR: (126 MHz, CDCl₃) δ 178.71, 166.04, 164.07, 162.70, 148.69, 145.39, 144.91, 135.63, 135.25, 129.69, 128.30, 128.14, 121.21, 119.69, 118.32, 106.58, 44.80, 39.60, 26.94, 21.66, 13.93, 10.62 ppm. HRMS: Calc'd for C₂₆H₂₆ClN₅O₄S₂Na [M+Na⁺] 594.1007; found: 594.0997.

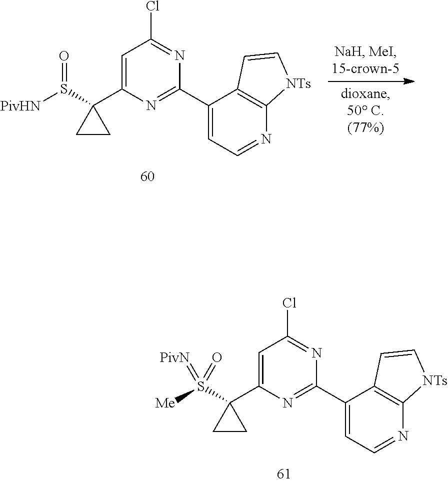

In a 20 mL septum capped vial equipped with a stir bar and argon balloon were added 60 (0.190 g, 0.332 mmol, 1 eq.), dioxane (3.3 mL) and 15-crown-5 ether (0.079 mL, 0.399 mmol, 1.2 eq.). NaH (0.016 g, 0.399 mmol, 1.2 eq., 60% wt) was added and the reaction mixture stirred at room temperature for 20 minutes. Mel (0.042 ml, 0.664 mmol, 2 eq.) was added and the reaction was heated to 50° C. for 24 hours. The reaction was cooled to room temperature and quenched with saturated aqueous NH₄Cl (10 mL) and water (5 mL). The aqueous layer was extracted with EtOAc (4×20 mL), combined organic layers were dried over Na₂SO₄, filtered and concentrated. Further purification by silica gel column chromatography using hexanes/EtOAc (0% to 30% EtOAc in hexanes) provided 61 (0.150 g, 0.256 mmol, 77% yield) as a light-yellow oil. TLC: R_{f}=0.41 (40% EtOAc in hexanes). ¹H NMR: (500 MHz, CDCl₃) δ 8.57 (d, J=5.2 Hz, 1H), 8.16 (d, J=5.1 Hz, 1H), 8.11-8.06 (m, 2H), 7.89 (d, J=4.0 Hz, 1H), 7.69 (s, 1H), 7.54 (d, J=4.0 Hz, 1H), 7.28 (d, J=8.5 Hz, 2H), 3.34 (s, 3H), 2.37 (s, 3H), 2.33 (ddd, J=10.4, 7.6, 5.9 Hz, 1H), 1.96 (ddd, J=10.7, 7.3, 5.2 Hz, 1H), 1.74 (ddd, J=9.7, 7.3, 5.8 Hz, 1H), 1.65 (ddd, J=9.6, 6.0, 3.8 Hz, 1H), 1.06 (s, 9H) ppm. ¹¹C NMR: (126 MHz, CDCl₃) δ 188.28, 164.63, 164.02, 162.48, 148.73, 145.41, 144.94, 135.65, 135.23, 129.70, 128.21, 128.17, 121.97, 121.26, 118.15, 106.52, 45.89, 41.43, 39.87, 27.49, 21.67, 14.23, 13.57 ppm. HRMS: Calc'd for C₂₇H₂₉ClN₅O₄S₂ [M+H⁺] 586.1344; found: 586.1348.

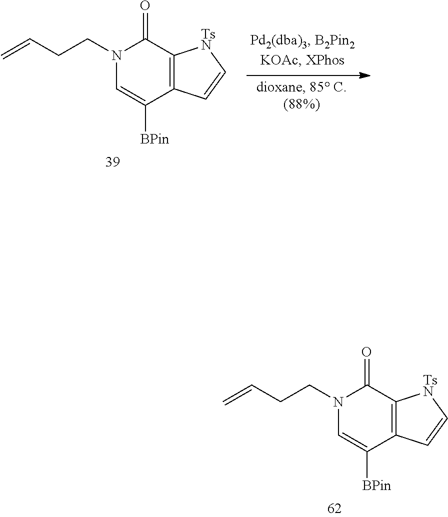

In a 100 mL round-bottomed flask equipped with a stir bar was 39 (2.82 g, 6.69 mmol, 1 eq.), B₂Pin₂ (3.4 g, 13.3 mmol, 2 eq.), KOAc (1.45 g, 14.7 mmol, 2.2 eq.), and dioxane (34 mL). The resulting mixture was degassed with argon while sonicating for 30 minutes. XPhos (0.319 g, 0.669 mmol, 0.1 eq.) and Pd₂(dba)₃ (153 mg, 0.167 mmol, 0.03 eq.) were added and the reaction mixture was degassed with argon while sonicating for an additional 15 minutes. The argon balloon was removed, and the reaction mixture was heated to 80° C. for 5 hours then cooled to room temperature and quenched with brine (70 mL) and extracted with EtAOc (4×75 mL). The combined organic layers were dried over Na₂SO₄, filtered and concentrated. Further purification by silica gel column chromatography using hexanes/EtOAc (0% to 20% EtOAc) provided 62 (2.77 g, 5.91 mmol, 88% yield) as a thick yellow oil that upon trituration with hexanes/Et₂O gives a colorless crystalline solid. TLC: R_{f}=0.68 (20% EtOAc in hexanes). ¹H NMR: (500 MHz, CDCl₃) δ 7.94 (d, J=8.4 Hz, 2H), 7.87 (d, J=3.4 Hz, 1H), 7.46 (s, 1H), 7.25 (d, J=7.9 Hz, 3H), 6.90 (d, J=3.4 Hz, 1H), 5.72 (ddt, J=17.2, 10.3, 6.8 Hz, 1H), 5.03-4.93 (m, 2H), 4.02-3.93 (m, 2H), 2.45-2.37 (m, 1H), 2.38 (s, 3H), 1.32 (s, 12H) ppm. ¹³C NMR: (126 MHz, CDCl₃) δ 153.33, 144.58, 141.28, 139.53, 136.30, 134.17, 130.93, 129.37, 128.44, 122.41, 117.47, 108.09, 83.74, 48.67, 33.81, 24.89, 21.66 ppm. HRMS: Calc'd for C₂₄H₃₀BN₂O₅S [M+H⁺]469.1963; found: 469.1960.

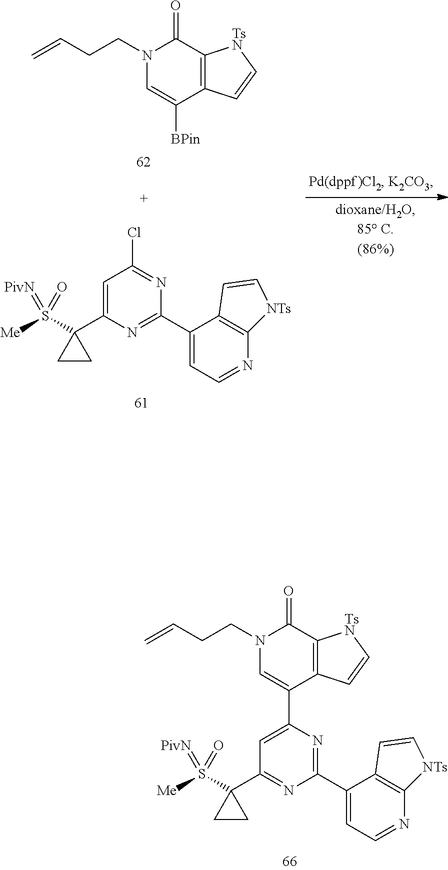

In a 1-dram septum capped reaction vial equipped with a stir bar and argon balloon were added 61 (0.165 g, 0.282 mmol, 1 eq.), 62 (0.145 g, 0.310 mmol, 1.1 eq.), dioxane (0.3 mL) and water (0.15 mL). K₂CO₃ (0.078 g, 0.560 mmol, 2 eq.) and Pd(PPh₃)₄(10.3 mg, 14.1 μmol) were added. The resulting reaction mixture was degassed by bubbling argon through the mixture while sonicating for 15 minutes then heated to 85° C. for 16 hours. The reaction was cooled to room temperature, quenched brine (15 mL) and extracted with EtOAc (4×20 mL). Combined organic layers were dried over Na₂SO₄, filtered and concentrated. Further purification by silica gel column chromatography using hexanes/EtOAc (10% to 60% EtOAc in hexanes) provided 66 (0.217 g, 0.243 mmol, 86% yield) as a golden foam. TLC: R_{f}=0.47 (60% EtOAc in hexanes). ¹H NMR: (500 MHz, CDCl₃) δ 8.61 (d, J=5.1 Hz, 1H), 8.19 (d, J=5.1 Hz, 1H), 8.12 (d, J=8.4 Hz, 2H), 8.04-7.99 (m, 3H), 7.86 (d, J=4.0 Hz, 1H), 7.83 (d, J=7.6 Hz, 2H), 7.50 (d, J=4.0 Hz, 1H), 7.32 (d, J=8.1 Hz, 2H), 7.29 (d, J=8.2 Hz, 2H), 7.19 (d, J=3.5 Hz, 1H), 5.81-5.70 (m, 1H), 5.03 (s, 1H), 5.02-4.99 (m, 1H), 4.16-4.04 (m, 2H), 3.39 (s, 3H), 2.49 (q, J=7.2 Hz, 2H), 2.42 (s, 3H), 2.38 (s, 3H), 2.29-2.23 (m, 1H), 2.04 (ddd, J=10.5, 7.3, 5.4 Hz, 1H), 1.86-1.80 (m, 1H), 1.59 (ddd, J=9.5, 7.7, 5.4 Hz, 1H), 1.00 (s, 9H) ppm. ¹³C NMR: (126 MHz, CDCl₃) δ 188.08, 163.55, 163.33, 163.02, 152.58, 148.66, 145.40, 145.17, 144.99, 137.64, 135.79, 135.30, 134.79, 133.71, 133.59, 131.77, 129.71, 129.51, 128.78, 128.22, 127.75, 123.13, 121.34, 118.20, 118.11, 117.52, 110.71, 106.58, 106.42, 49.42, 46.56, 41.41, 39.99, 33.71, 27.55, 21.73, 21.68, 13.55, 13.42 ppm.

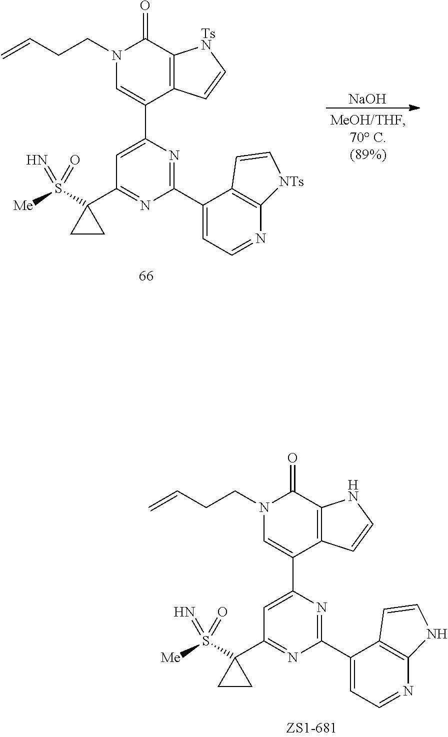 [SMILES: C=CCCn1cc(-c2cc(C3([S@](C)(=N)=O)CC3)nc(-c3ccnc4c3ccn4S(=O)(=O)c3ccc(C)cc3)n2)c2ccn(S(=O)(=O)c3ccc(C)cc3)c2c1=O ; "NaOH" ; C=CCCn1cc(-c2cc(C3([S@](C)(=N)=O)CC3)nc(-c3ccnc4[nH]ccc34)n2)c2cc[nH]c2c1=O]

In a 2-dram septum capped reaction vial equipped with a stir bar were added 66 (80.0 mg, 89.7 μmol, 1 eq.), THF (0.6 mL) and MeOH (0.4 mL). An aqueous 1 M NaOH solution (0.448 mL, 0.897 mmol, 10 eq.) was added then heated to 70° C. for 4 hours. The reaction was cooled to room temperature and directly adsorbed to silica gel (diluted with MeOH). Purification by silica gel column chromatography using DCM/MeOH (0% to 5% MeOH in DCM) to provide ZS1-681 (40 mg, 80.1 μmol, 89% yield) as a golden foam. TLC: R_{f}=0.18 (5% MeOH in DCM). ¹H NMR: (500 MHz, DMSO) δ 12.28 (s, 1H), 11.89 (s, 1H), 8.44 (d, J=5.0 Hz, 1H), 8.33 (s, 1H), 8.14 (s, 1H), 8.13 (d, J=5.0 Hz, 1H), 7.66-7.62 (m, 1H), 7.48 (t, J=2.7 Hz, 1H), 7.36 (dd, J=3.3, 2.0 Hz, 1H), 7.13-7.10 (m, 1H), 5.91 (ddt, J=17.0, 10.3, 6.7 Hz, 1H), 5.09 (dd, J=17.2, 1.7 Hz, 1H), 5.05 (dd, J=10.3, 1.6 Hz, 1H), 4.24 (t, J=7.1 Hz, 2H), 4.07-4.04 (m, 1H), 3.13-3.10 (m, 3H), 2.56 (q, J=7.0 Hz, 2H), 1.93-1.87 (m, 1H), 1.69-1.62 (m, 3H) ppm. ¹³C NMR: (126 MHz, DMSO) δ 165.29, 164.00, 163.71, 154.51, 150.73, 142.97, 136.59, 135.48, 132.77, 128.29, 128.19, 127.35, 123.86, 118.32, 117.83, 117.06, 115.16, 111.68, 104.41, 102.28, 48.41, 47.70, 41.49, 34.09, 13.15, 12.33 ppm. HRMS: Calc'd for C₂₆H₂₅N₇O₂S [M+H⁺] 500.1863; found: 500.1860.

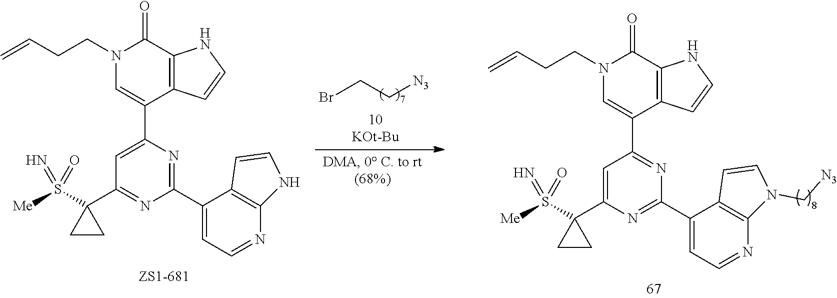

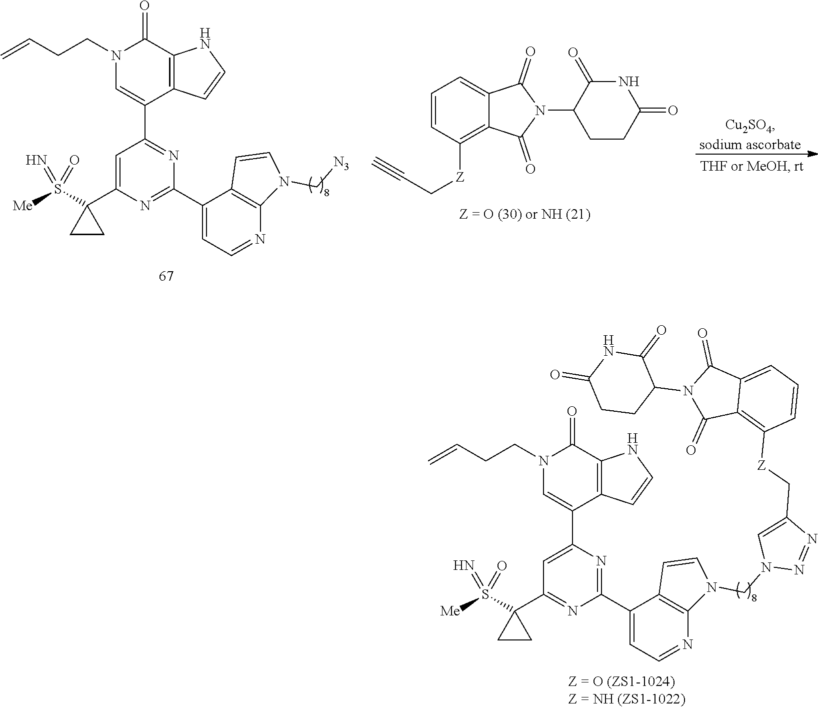

### Synthetic Scheme for the Synthesis of ZS1-681 PROTACs.

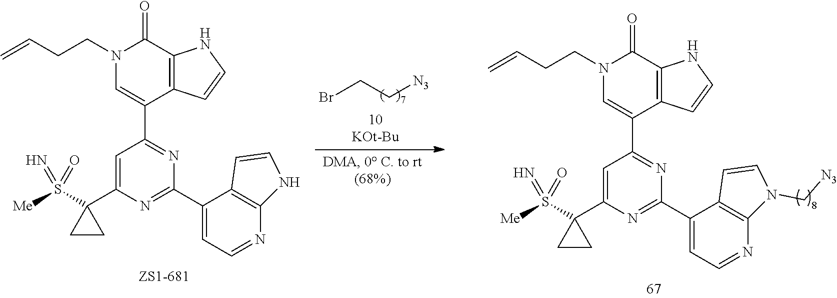

In a 1-dram septum capped reaction vial equipped with a stir bar and argon balloon was ZS1-681 (18 mg, 36 μmol, 1 eq.) in DMA (0.5 mL) at 0° C. KOt-Bu (5.2 mg, 22 μmol, 1.3 eq.) was added then stirred at 0° C. for 15 minutes before the addition of 10 (9.3 mg, 39.6 μmol, 1.1 eq.). The reaction stirred at 0° C. for 2 hours then quenched with saturated aqueous NH₄Cl (5 mL) and extracted with EtOAc (4×15 mL). The combined organic layers were dried over Na₂SO₄, filtered and concentrated. Further purification by silica gel column chromatography using DCM/MeOH (0% to 4% MeOH) provided XX (16 mg, 24.5 mol, 68% yield) as a yellow oil. TLC: R_{f}=0.30 (5% MeOH in DCM). ¹H NMR: (500 MHz, CDCl₃) δ 10.64 (s, 1H), 8.53 (d, J=5.1 Hz, 1H), 8.24 (d, J=1.3 Hz, 1H), 8.23 (d, J=5.2 Hz, 1H), 8.05 (d, J=0.9 Hz, 1H), 7.44 (d, J=3.4 Hz, 1H), 7.41-7.38 (m, 2H), 7.04 (t, J=2.4 Hz, 1H), 5.95-5.84 (m, 1H), 5.17-5.08 (m, 2H), 4.42 (t, J=7.2 Hz, 2H), 4.30-4.22 (m, 2H), 3.23 (t, J=6.9 Hz, 2H), 3.20 (s, 3H), 2.64 (q, J=6.8 Hz, 2H), 2.00-1.90 (m, 4H), 1.80-1.73 (m, 2H), 1.56 (q, J=7.1 Hz, 2H), 1.41-1.30 (m, 9H) ppm. ¹³C NMR: (126 MHz, CDCl₃) δ 164.25, 163.87, 163.44, 156.28, 154.86, 143.67, 134.06, 131.44, 129.91, 127.80, 127.65, 127.63, 124.11, 118.10, 117.56, 115.32, 113.94, 112.89, 104.10, 101.72, 68.67, 51.43, 48.93, 48.46, 45.26, 41.74, 41.09, 34.10, 31.03, 30.45, 28.99, 28.78, 26.74, 13.46 ppm. HRMS: Calc'd for C₃₄H₄₁N₁₀O₂S [M+H⁺] 653.3129; found: 653.3121.

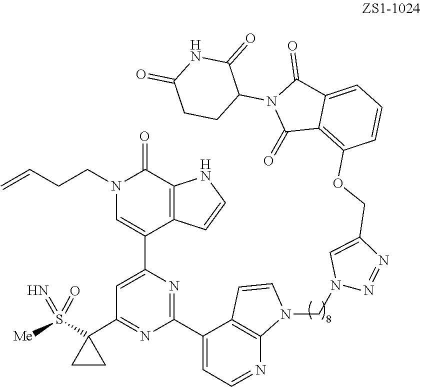

The title compound was prepared using GP-2.

67 (10 mg, 15 μmol, 1 eq.), 30 (4.8 mg, 15 μmol, 1 eq.), sodium ascorbate (0.60 mg, 3.1 μmol, 0.2 eq.), CuSO₄ (0.50 mg, 3.1 μmol, 0.2 eq.), THF (0.25 mL) and H₂O (30 μL) were used. Purification by preparative TLC (6% MeOH in DCM) provided ZS1-1024 (9 mg, 9.3 μmol, 61% yield as a white solid. ¹H NMR: (500 MHz, DMSO) δ 12.34-12.14 (m, 1H), 11.09 (s, 1H), 8.47 (d, J=5.0 Hz, 1H), 8.33 (s, 1H), 8.24 (s, 1H), 8.16-8.10 (m, 2H), 7.84-7.79 (m, 1H), 7.75-7.68 (m, 2H), 7.49-7.44 (m, 2H), 7.35 (d, J=3.4 Hz, 1H), 7.11 (t, J=2.4 Hz, 1H), 5.90 (ddt, J=16.9, 10.1, 6.6 Hz, 1H), 5.39 (s, 2H), 5.13-5.01 (m, 3H), 4.36-4.30 (m, 4H), 4.24 (t, J=7.1 Hz, 2H), 4.02-3.92 (m, 2H), 3.51-3.46 (m, 5H), 2.86 (ddd, J=17.0, 14.0, 5.4 Hz, 1H), 2.60-2.52 (m, 3H), 1.96-1.91 (m, 1H), 1.83-1.76 (m, 4H), 1.30-1.19 (m, 1OH) ppm. HRMS: Calc'd for C₅₀H₅₃N₁₂O₇S [M+H⁺]965.3875; found: 965.3867.

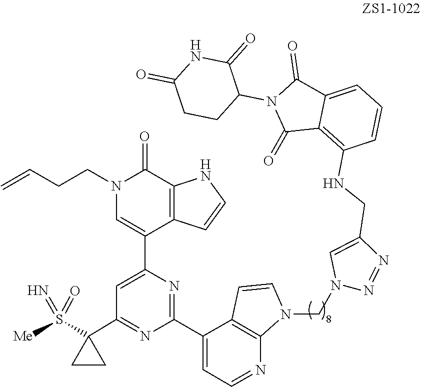

The title compound was prepared using GP-2.

67 (9.0 mg, 14 μmol, 1 eq.), 21 (4.3 mg, 14 μmol, 1 eq.), sodium ascorbate (0.60 mg, 2.8 μmol, 0.2 eq.), CuSO₄ (0.50 mg, 2.8 μmol, 0.2 eq.), THF (0.25 mL) and H₂O (30 L) were used. An aqueous work up using H₂O (4 mL) and extracting with EtOAc (3×15 mL) and DCM (2×10 mL), drying combined organic layers over Na₂SO₄, filtering and concentrating provided a crude oil that was purified by preparative TLC (6% MeOH in DCM) provided ZS1-1022 (3.5 mg, 3.6 μmol, 26% yield) as a yellow oil. ¹H NMR: (500 MHz, DMSO) δ 12.28 (s, 1H), 11.09 (s, 1H), 8.47 (d, J=5.0 Hz, 1H), 8.33 (s, 1H), 8.15-8.14 (m, 2H), 7.99 (s, 1H), 7.73 (d, J=3.5 Hz, 1H), 7.58-7.52 (m, 2H), 7.48 (t, J=2.8 Hz, 2H), 7.36 (d, J=3.4 Hz, 1H), 7.11 (t, J=2.5 Hz, 1H), 5.94-5.87 (m, 2H), 5.34-5.33 (m, 3H), 5.08-5.02 (m, 4H), 4.58-4.56 (m, 2H), 4.33-4.24 (m, 2H), 4.13-4.06 (m, 2H), 3.18-3.10 (m, 5H), 2.86 (m, 3H), 1.71-1.65 (m, 4H), 1.47 (m, 2H), 1.31-1.25 (m, 10H) ppm. HRMS: Calc'd for C₅₀H₅₄N₁₃O₆S [M+H⁺] 964.4035; found: 964.4006.

### REFERENCES

1. Shultz, Z. P.; Scattolin, T.; Wojtas, L.; Lopchuk, J. M., Nature Synthesis 2022,
2. Foote, K. M.; Nissink, J. W. M.; Turner, P. Morpholinopyrimidines as ATR kinase inhibitors and their preparation, pharmaceutical compositions and use in the treatment of cancer. WO2011154737, 2011.
3. Botta, M.; Maccari, G.; Sanfilippo, S.; De Luca, F.; Docquier, J.-D.; Deodato, D. Preparation of linear guanidine derivatives as antibacterial agents. WO2016055644, 2016.
4. Li, Z.; Kosuri, S.; Foster, H.; Cohen, J.; Jumeaux, C.; Stevens, M. M.; Chapman, R.; Gormley, A. J., J. Am. Chem. Soc. 2019, 141 (50), 19823-19830.
5. Qi, J.; Armstrong, S.; Wu, L. Compounds, compositions, and methods for targeted protein degradation of SMARCA2 and SMARCA4. WO2020264172, 2020.
6. Phillips, A. J.; Nasveschuk, C. G.; Henderson, J. A.; Liang, Y.; Fitzgerald, M. E.; Michael, R. E. Preparation of bromodomain targeting degronimers for target protein degradation. WO2017197056, 2017.
7. Yang, K.; Song, Y.; Xie, H.; Wu, H.; Wu, Y.-T.; Leisten, E. D.; Tang, W., Bioorganic & Medicinal Chemistry Letters 2018, 28 (14), 2493-2497.
8. Neufeld, J.; Daniliuc, C. G.; Gilmour, R., Beilstein Journal of Organic Chemistry 2020, 16, 1627-1635.
9. Wu, H.; Yang, K.; Zhang, Z.; Leisten, E. D.; Li, Z.; Xie, H.; Liu, J.; Smith, K. A.; Novakova, Z.; Barinka, C.; Tang, W., J. Med. Chem. 2019, 62 (15), 7042-7057.
10. DeLomba, W. C.; Stone, E. A.; Alley, K. A.; Iannarone, V.; Tarsis, E.; Ovaska, S.; Ovaska, T. V., J. Org. Chem. 2020, 85 (15), 9464-9474.
11. Burnette, P.; Lawrence, N. J.; Lawrence, H. Preparation of dimeric compounds as immuno-modulators against cereblon-based mechanisms. WO2020014489, 2020.
12. Song, Y.; Li, A.; Li, H.; Li, X.; Yang, J. Preparation of DNA therapeutic agents and conjugates thereof. WO2021178818, 2021.
13. Crawford, T. D.; Tsui, V.; Flynn, E. M.; Wang, S.; Taylor, A. M.; Côté, A.; Audia, J. E.; Beresini, M. H.; Burdick, D. J.; Cummings, R.; Dakin, L. A.; Duplessis, M.; Good, A. C.; Hewitt, M. C.; Huang, H.-R.; Jayaram, H.; Kiefer, J. R.; Jiang, Y.; Murray, J.; Nasveschuk, C. G.; Pardo, E.; Poy, F.; Romero, F. A.; Tang, Y.; Wang, J.; Xu, Z.; Zawadzke, L. E.; Zhu, X.; Albrecht, B. K.; Magnuson, S. R.; Bellon, S.; Cochran, A. G., J. Med. Chem. 2016, 59 (11), 5391-5402.
14. Wang, S.; Tsui, V.; Crawford, T. D.; Audia, J. E.; Burdick, D. J.; Beresini, M. H.; Cote, A.; Cummings, R.; Duplessis, M.; Flynn, E. M.; Hewitt, M. C.; Huang, H.-R.; Jayaram, H.; Jiang, Y.; Joshi, S.; Murray, J.; Nasveschuk, C. G.; Pardo, E.; Poy, F.; Romero, F. A.; Tang, Y.; Taylor, A. M.; Wang, J.; Xu, Z.; Zawadzke, L. E.; Zhu, X.; Albrecht, B. K.; Magnuson, S. R.; Bellon, S.; Cochran, A. G., J. Med. Chem. 2018, 61 (20), 9301-9315.
15. Demont, E. H.; Redshaw, S.; Walter, D. S. Tricyclic indole derivatives for use in the treatment of Alzheimer's disease. WO2006040148, 2006.
16. Parra, J.; Mercader, J. V.; Agullo, C.; Abad-Fuentes, A.; Abad-Somovilla, A., Tetrahedron 2011, 67 (3), 624-635.

TABLE 1
TAF1 PROTACs induce apoptosis preferentially
in hematopoietic tumor cell lines

| | | | Apoptosis | Apoptosis |
|---|---|---|---|---|
| Cell line | Cell type | p53 status | ZS1-958 | ZS3-025 |
| NB4 | AML | R248Q | + | + |
| U937 | AML | 59fs | + | + |
| Molm13 | AML | WT | − | + |
| Kasumi-1 | AML | R248Q | + | + |
| MV-4-11 | AML | WT | + | + |
| UKE1 | AML | WT | + | + |
| SET2 | AML | R248W | + | |
| HL60 | AML | Null | + | + |
| LAMA | CML | 319stop | − | + |
| Jurkat | TALL | 196stop | + | + |
| Loucy | TALL | V272M | + | |
| GA10 | B lymphoma | P152L | − | |
| ST486 | B lymphoma | R158H | − | |
| SU-DHL-6 | B lymphoma | Y234C | − | + |
| U2OS | Osteosarcoma | WT | | − |
| Saos2 | Osteosarcoma | Null | | + |
| T47D | Breast cancer | L194F | | − |
| HCT116 | Colon cancer | WT | − | |
| H1299 | Lung cancer | Null | − | |
| A549 | Lung cancer | WT | − | |
| UC3 | Bladder cancer | F113C | − | − |
| J82 | Bladder cancer | E271K | − | − |
| HT1376 | Bladder cancer | P250L | | + |
| 5637 | Bladder cancer | R280T | | + |

Cells were treated with 200 nM ZS1-958 or 50 nM ZS3-025 for 48-96 hrs for analysis of cell death by morphological observation and Western blot for PARP cleavage to confirm apoptosis. Most hematopoietic tumor cell lines and a fraction of solid tumor cell lines undergo apoptosis after TAF1 PROTAC treatment. Untested entries are left blank.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

This listing of claims replaces all prior versions and listings of claims in the application:

1. A compound of Formula I, Formula II, or Formula III: 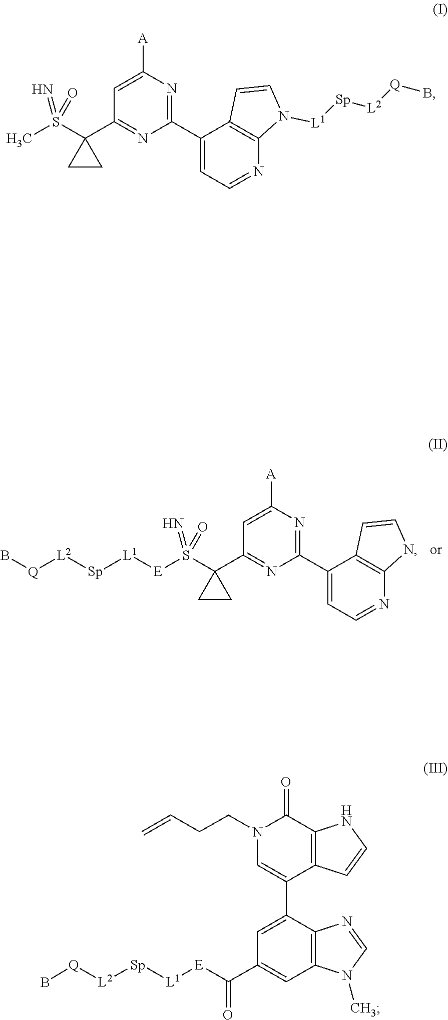
or a pharmaceutically acceptable salt thereof; wherein:
A is selected from: 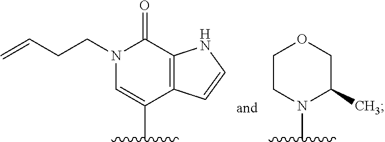 [SMILES: *c1cn(CCC=C)c(=O)c2[nH]ccc12 ; *N1CCOC[C@H]1C]
B is an E3 ubiquitin ligase-recruiting moiety;
L¹ and L² are independently selected from C₁-C₁₀ alkyl, C₂-C₁₀ alkenyl, C₂-C₁₀ alkynyl, and 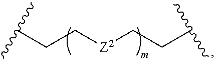
each of which may be substituted with one or more groups independently selected from X as allowed by valency;
Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl, 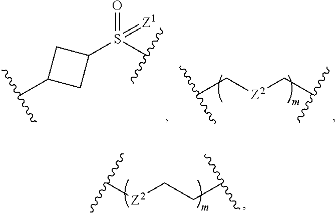
each of which may be optionally substituted with one or more groups independently selected from X as allowed by valency;
m is independently selected at each occurrence from 1, 2, 3, 4, 5, 6, 7, 8, 9, or 10;
Q is a bond or selected from 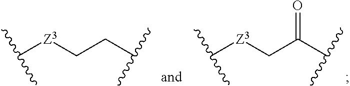
E is a bond or selected from —N(R^{a})— and 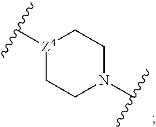
Z¹ and Z² are independently selected at each occurrence from 0 or N(R^{a});
Z³ is selected from CH₂, O, N(R^{a}), and S;
Z⁴ is selected from CH and N;
X is independently selected at each occurrence from halo, cyano, azido, C₁-C₆ alkyl, C₁-C₆haloalkyl, C₂-C₆ alkenyl, C₂-C₆ alkynyl, (C₃-C₆ cycloalkyl)(C₀-C₃ alkyl)-, (3- to 6-membered heterocyclyl)—(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{Y} are independently selected at each occurrence from hydrogen, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)—(C₀-C₃ alkyl)-, (5-to 10-membered monocyclic or bicyclic heteroaryl)—(C₀-C₃ alkyl)-, each of which may be optionally substituted with one or more groups selected from Y as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)—(C₀-C₃ alkyl)-, (5-to 10-membered monocyclic or bicyclic heteroaryl)—(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

2-6. (canceled)

7. A compound of Formula IV 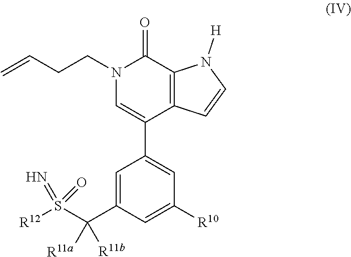
or a pharmaceutically acceptable salt thereof;
wherein:
R¹⁰ is selected from R^{10a} and —NH—R^{10a};
R^{10a} is selected from aryl, heteroaryl, cycloalkyl, or heterocycloalkyl, each of which may be optionally substituted with one or more groups independently selected from X as allowed by valency;
R^{11a} and R^{11b} are brought together with the carbon to which they are attached to form a cycloalkyl or heterocycloalkyl ring which may be optionally substituted with one or more groups independently selected from X as allowed by valency;
R¹² is selected from R^{12a} and —NH—R^{12b};
R^{12a} is selected from C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;
R^{12b} is selected from hydrogen C₁-C₆ alkyl, —(C₀-C₆ alkyl)-(aryl), —(C₀-C₆ alkyl)-(heteroaryl), —(C₀-C₆ alkyl)-(cycloalkyl), and —(C₀-C₆ alkyl)-(heterocycloalkyl), each of which may be optionally substituted with one or more groups independently selected from X as allowed by valency;
wherein at least one of R¹⁰ or R¹² is substituted with B-Q-L²-Sp-L¹-E-;
B is an E3 ubiquitin ligase-recruiting moiety;
L¹ and L² are independently selected from C₁-C₁₀ alkyl, C₂-C₁₀ alkenyl, C₂-C₁₀ alkynyl, and 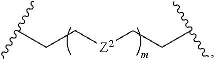
each of which may be substituted with one or more groups independently selected from X as allowed by valency;
Sp is selected from C₁-C₁₀ alkyl, C₂-C₁₀ alkenyl, C₂-C₁₀ alkynyl, 3- to 6-membered cycloalkyl, 3- to 6-membered heterocyclyl, aryl, 5- to 6-membered heteroaryl, 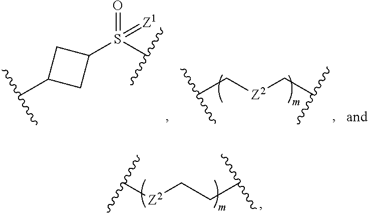
each of which may be optionally substituted with one or more groups independently selected from X as allowed by valency;
m is independently selected at each occurrence from 1, 2, 3, 4, 5, 6, 7, 8, 9, or 10;
Q is selected from 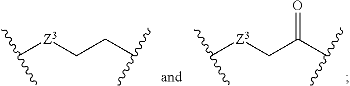
E is absent or selected from —N(R^{a})— and 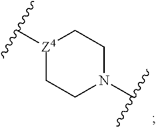
Z¹ and Z² are independently selected at each occurrence from O or N(R^{a});
Z³ is selected from CH₂, O, N(R^{a}), and S;
Z⁴ is selected from CH and N;
X is independently selected at each occurrence from halo, cyano, azido, C₁-C₆ alkyl, C₁-C₆haloalkyl, C₂-C₆ alkenyl, C₂-C₆ alkynyl, (C₃-C₆ cycloalkyl)(C₀-C₃ alkyl)-, (3- to 6-membered heterocyclyl)—(C₀-C₃ alkyl)-, (aryl)(C₀-C₃ alkyl)-, (5- to 6-membered heteroaryl)(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted with one or more groups selected from Y as allowed by valency;
R^{a} is hydrogen or C₁-C₆ alkyl;
R^{x} and R^{Y} are independently selected at each occurrence from hydrogen, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)—(C₀-C₃ alkyl)-, (5-to 10-membered monocyclic or bicyclic heteroaryl)—(C₀-C₃ alkyl)-, each of which may be optionally substituted with one or more groups selected from Y as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)—(C₀-C₃ alkyl)-, (4- to 6-membered heterocycle)—(C₀-C₃ alkyl)-, (5- to 10-membered monocyclic or bicyclic aryl)—(C₀-C₃ alkyl)-, (5-to 10-membered monocyclic or bicyclic heteroaryl)—(C₀-C₃ alkyl)-, —OR^{x}, —SR^{x}, and —NR^{x}R^{y}, each of which may be optionally substituted with one or more groups selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

8-12. (canceled)

13. The compound of claim 7, wherein R¹⁰ is selected from:

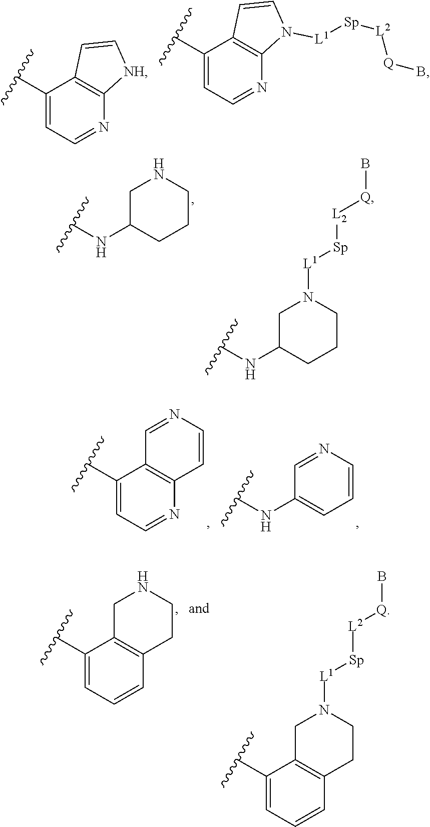

14-15. (canceled)

16. The compound of claim 7, wherein R^{11a} and R^{11b} are brought together with the carbon to which they are attached to form:

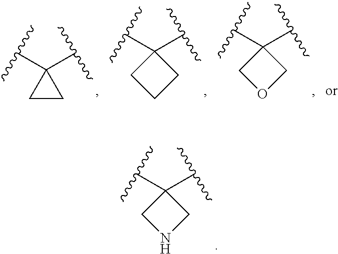 [SMILES: *C1(*)CC1 ; *C1(*)CCC1 ; *C1(*)COC1 ; *C1(*)CNC1]

17. (canceled)

18. The compound of claim 7, wherein R^{12a} is C₁-C₆ alkyl or —(C₁-C₆alkyl)-(substituted or unsubstituted aryl).

19-21. (canceled)

22. The compound of claim 7, wherein R^{12b} is hydrogen —(C₁-C₆ alkyl)-(substituted or unsubstituted aryl).

23. (canceled)

24. The compound of claim 7, wherein R¹² is selected from:

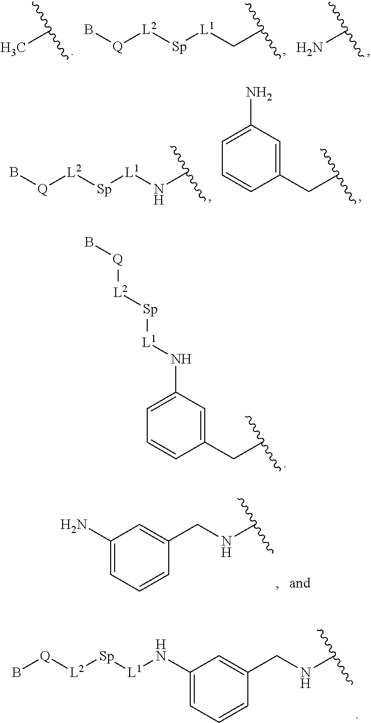

25-29. (canceled)

30. The compound of claim 1, wherein L¹ is C₁-C₁₀ alkyl.

31. The compound of claim 1, wherein L² is C₁-C₁₀ alkyl.

32. The compound of claim 1, wherein Sp is 5- to 6-membered heteroaryl or 3- to 6-membered heterocyclyl.

33. The compound of claim 1, wherein Sp is

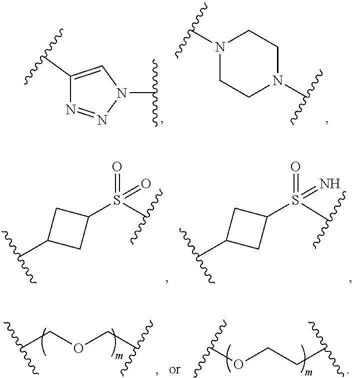

34-41. (canceled)

42. The compound of claim 1, wherein Z³ is selected from CH₂, O, and NH.

43. The compound of claim 1, wherein B is selected from 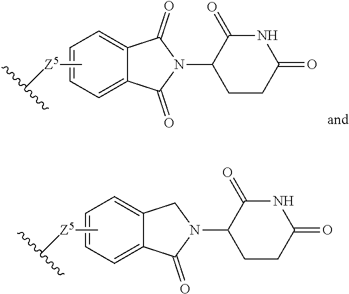
wherein Z⁵ is selected from O, N(R^{a}), and CH₂, or wherein B is selected from

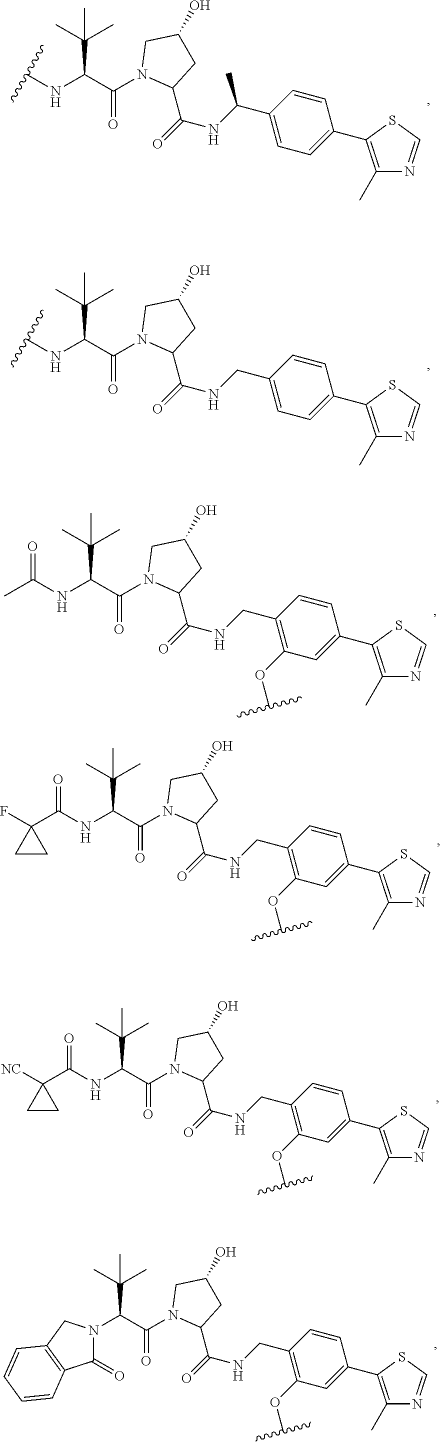 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

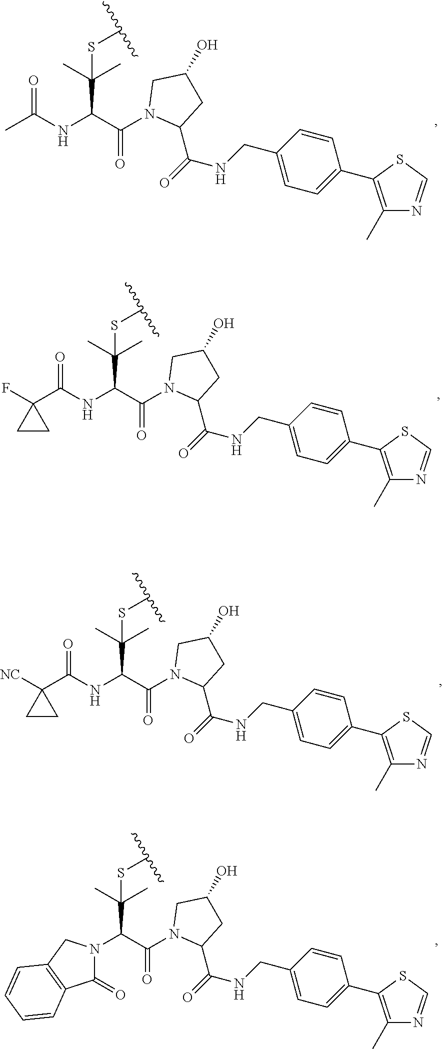 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O]

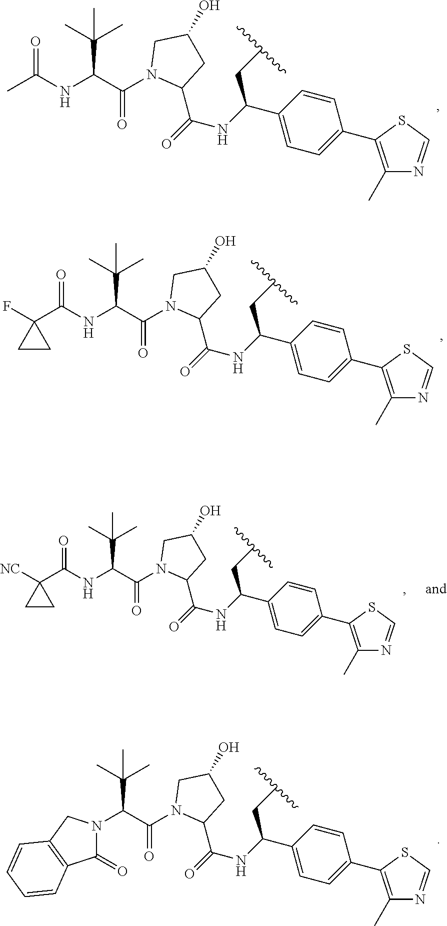 [SMILES: *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)C)c1ccc(-c2scnc2C)cc1 ; *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

44. (canceled)

45. A compound selected from: 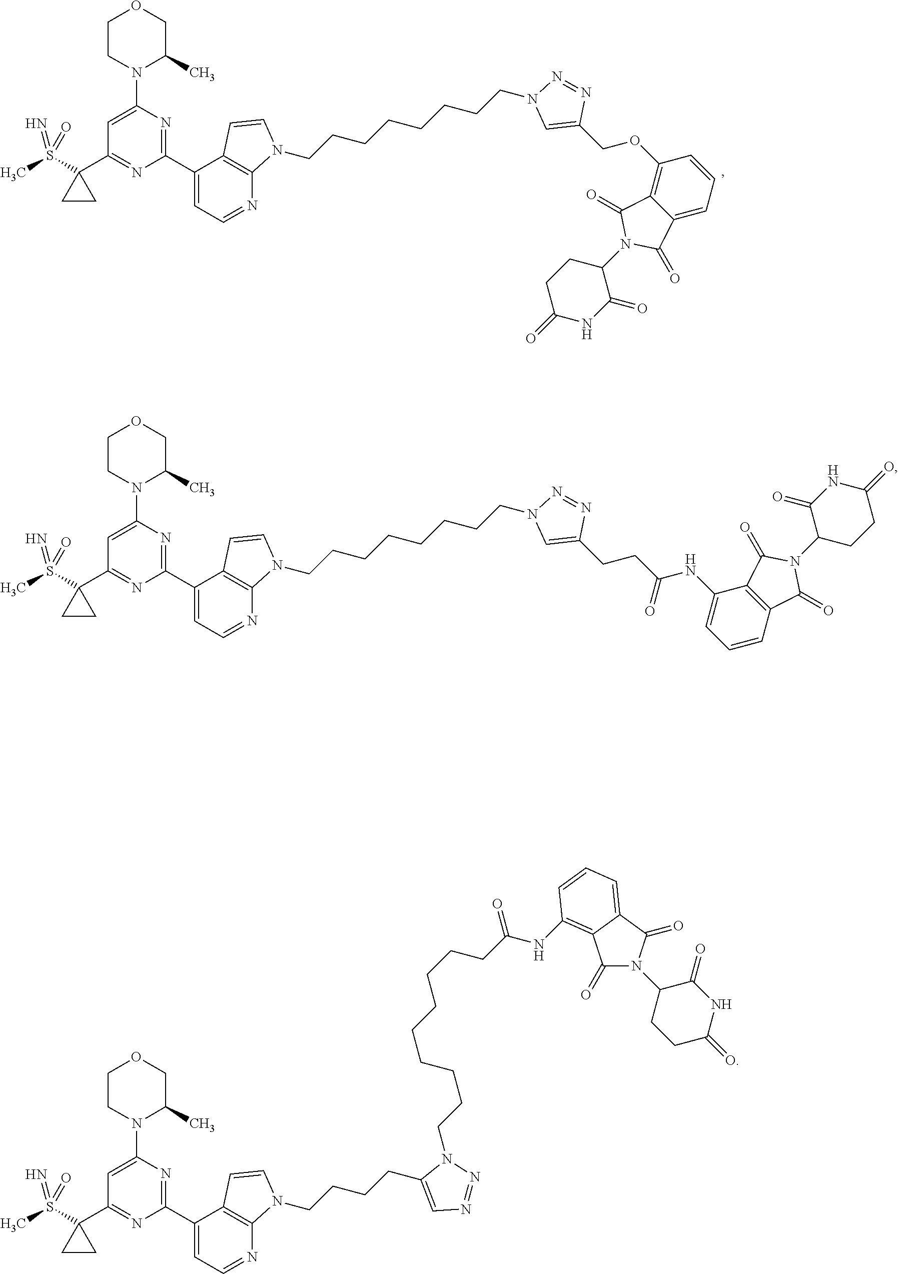 [SMILES: C[C@@H]1COCCN1c1cc(C2([S@](C)(=N)=O)CC2)nc(-c2ccnc3c2ccn3CCCCCCCCn2cc(COc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCCCCCn2cc(CCC(=O)Nc3cccc4c3C(=O)N(C3CCC(=O)NC3=O)C4=O)nn2)n1 ; C[C@@H]1COCCN1c1cc(C2([S@](C)(=N)=O)CC2)nc(-c2ccnc3c2ccn3CCCCc2cnnn2CCCCCCCCCC(=O)Nc2cccc3c2C(=O)N(C2CCC(=O)NC2=O)C3=O)n1] 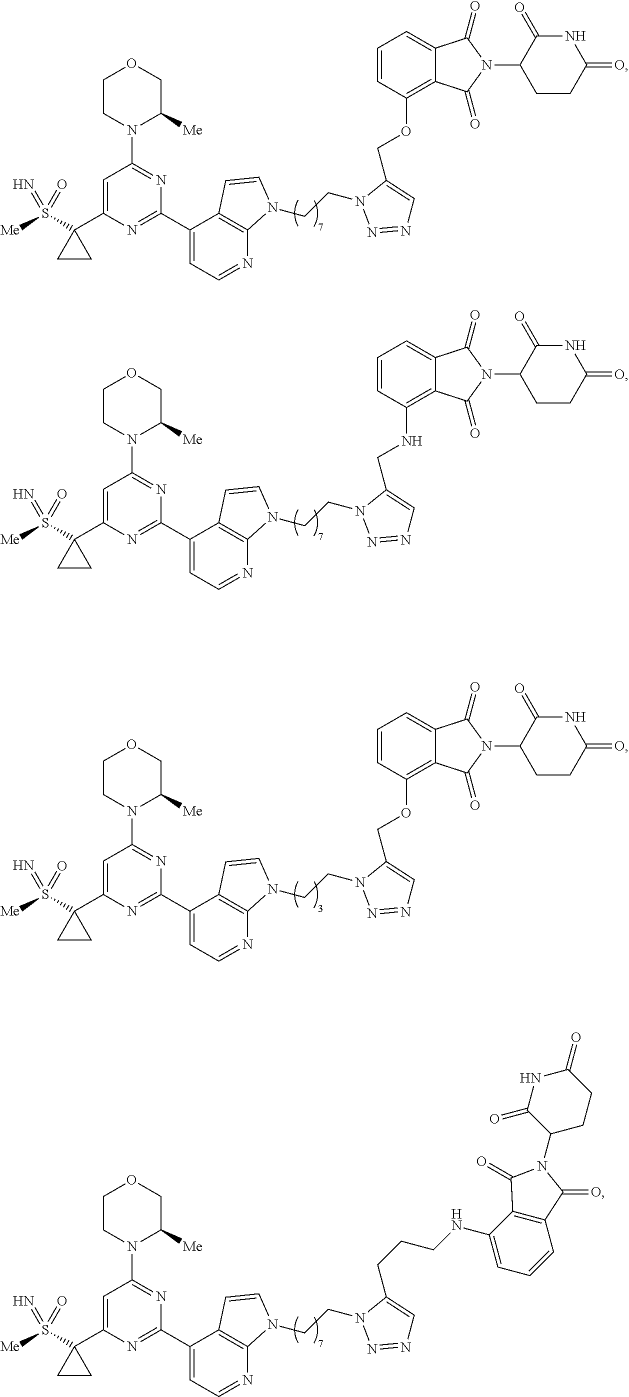 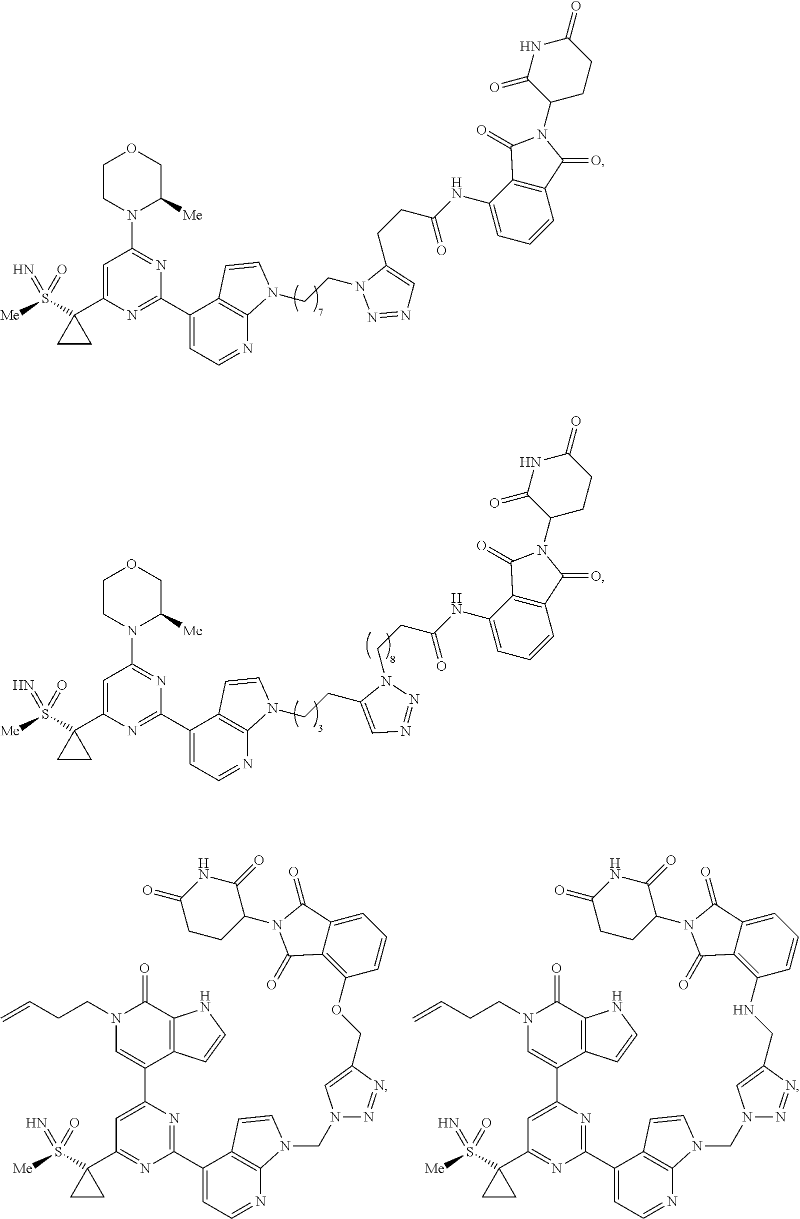 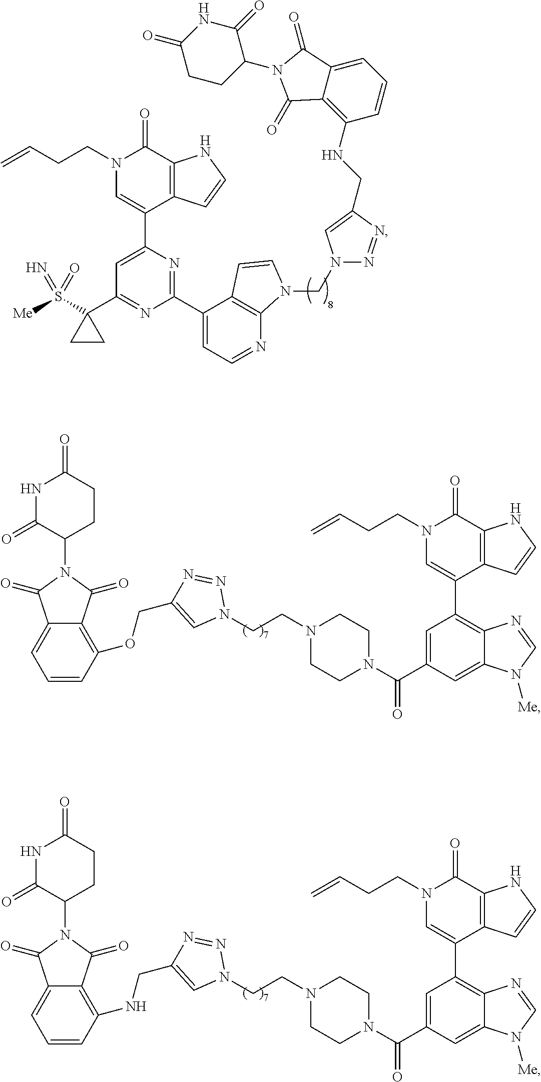 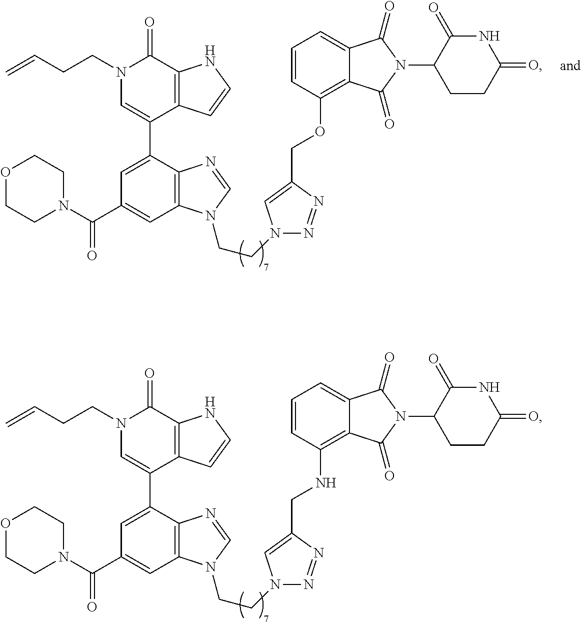
or a pharmaceutically acceptable salt thereof.

46. (canceled)

47. A pharmaceutical composition comprising a compound of claim 1, or a pharmaceutically acceptable salt thereof, and a pharmaceutically acceptable carrier.

48. A method of treating cancer in a subject in need thereof comprising administering to the subject a therapeutically effective amount of a compound of claim 1, or a pharmaceutically acceptable salt thereof.

49. A pharmaceutical composition comprising a compound of claim 7, or a pharmaceutically acceptable salt thereof, and a pharmaceutically acceptable carrier.

50. A method of treating cancer in a subject in need thereof comprising administering to the subject a therapeutically effective amount of a compound of claim 7, or a pharmaceutically acceptable salt thereof.

51. A pharmaceutical composition comprising a compound of claim 45, or a pharmaceutically acceptable salt thereof, and a pharmaceutically acceptable carrier.

52. A method of treating cancer in a subject in need thereof comprising administering to the subject a therapeutically effective amount of a compound of claim 45, or a pharmaceutically acceptable salt thereof.
